SEQUENCE LISTING (63 sequences)

SEQ ID NO: 1 - DNA, 886 bp, Artificial Sequence
  lncGRS-1 (CTC-338M12.4)
cggccgcctttcccgttgggcacccagctgtggctgtgccctgtcctcgcccagctcccggcataggcatgcgctctgggccggggccggggccccccgcctccccgggaggaatcgctgggttgactctgggtcagcaaagggagcccggccctttctagcggggggacgtcctgcagccgctggaggaggctgagtcacgcttttcccaccagaagtctacctgtgcctgcgcccgccggacctgccggcctcttccttctcatccagggggcctcttctccagcctcacggaggtgggaagcttgctggaggcccctacctgccttttggggaagaccccgaggctggcccacgccctgctggcaggacccttcctgactgggtccaccgttcttctgggtctcaccacttctcccagcccctgggctgcttcctctctcccgtgtctgggaccccagaggcaccacttccattccatacaagcttgcagagtcaagacgaactctaactcatgggatggacaaactggaagatgtaaaataagtaaggctttctgggccaaaaagcctcttcttacagaaaatcaaattttaaaagaacattgacctcaaaacaataaaactgtcctggttatgcaatagaaatagctatataaatggaatcatatccttaatgaacacctcctgtggctacccaacttttcacacatcttactgattataggccttgctgtgaatgctttcttgctctatctgaagtattcccaccagagttgtttccaactttatttcctatattatgaaaaatgaagtttttttaacattgtaacaaagtatatgaaagataatgtatactgtattaaataaaccattatacaaaaata

SEQ ID NO: 2 - DNA, 371 bp, Artificial Sequence
  lncGRS-2 (AC005624.2)
ctgggacttggagtccagtccctgggggtcccgcggggatggctctgggcacccgaagcccctccccagcgccgagacaggagggctggggtagattcgagaccccggtttgcagagcgcaagactgaggcccagggccgcgcagtcaggcaggaaacggcgctcctggcggatggaacagcacgggcaaaggctgggtatggggggtctcgctccgttgcccacactggagtgcagtggcgcaatcatggctgactgcagacttgaactcctggatgcaagccattctcctgcctcagcctcccgaggagctgggactacaggtgtgtgccagcacatgtggctaattattaaaaatttttttggctggg

SEQ ID NO: 3 - DNA, 4298 bp, Artificial Sequence
  lncGRS-3 (PDXDC2P)
ctcaaccatcaggttcggcagccggcggcgccgcctggcagctcctcctcttctccgccccgctggccgcgggcgcgggggacgtcagcgctgccagcgtggaaagagctgcggggcgcgggaggaggaagtagagcccgggaccgccgggccaccaccggccgcctcagccatggacgcgtccctggagaagatagcagaccccacgttagctgaaatgggaaaaaacttgaaggaggcagtgaagatgctggaggacagtcagagaagaacagaagaggaaaatggaaagaagctcatatccagagatattccaggcccactccagggcagtggacaagatatggtgagcatcctccagttagttcagaatctgatgcatggagatgaagatgaggagccccagagccccagaatccaaaatattggagaacaaggtcatgtggctgtgttgggacatagtctgggagcttatattttgactctggacgaagagaagctgagaaaacttacaactaggatactttcagataccaccttatggctatgcagaattttcagatatgaaaatgggtgtgcttatttccacgaagaggaaagagaaggacttgcaaagatatgtaggcttgccattcattctcaatatgaagacttcgtagtggatggcttcagtgggttatataacaagaagcctgtcatatatcttagtgctgctgctagacctggcctgggccaatacctttgtaatcagctcggcttgcccttcccctgcttgtgccgtgtaccctgtaacactatgtttggatcccagcatcagatggatgttgccttcctggagaaactgattaaagatgatatagagcgaggaagactgcccctgttgcttgtcgcaaatgcaggaacggcagcagtaggacacacagacaagattgggagattgaaagaactctgtgagcagtatggcatatggcttcatgtggagggtgtgaatctggcaacattggctctgggttatgtctcctcatcagtgctggctgcagccaaatgtgatagcatgacgatgactcctggcccgtggctgggtttgccagctgttcctgcggtgacactgtataaacacgaccctgccttgactttagttgctggtcttatatcaaataagcccacagacaaactccgtgccctgcctctgtggttatctttacaatacttgggacttgatgggtttgtggagaggatcaagcatgcctgtcaactgagtcaatggttgcaggaaagtttgaagaaagtgaattacatcaaaatcttggtggaagatgagctcagctccccagtggtggtgttcagatttttccaagaattaccaggctcagatccagtgtttaaagccgtcccagtgcccaacatgacaccttcagcagtcggccgggagaggcactcgtgtgatgcgctgaatctctggctgggagaacagctgaagcagctggtgcctgcgagcggcctcacagtcatggatctggaagctgagggcacgtgtttgcggttcagccctttgatgaccgcagcaggtatgatctcgtgaaaccttgagagaaactgaatgaggaatgaaactattgttcctgtttcacacagaagaaaactgaggttggcactcatgatgagcccctgttctcattctgcaaatggtgaagctctctatcgtcctgaccccacagttcctgtcccatgaccagggcccgctcaccaaggagctgcagcagcacgtaaagtcagtgacatgcccatgcgagtacctgaggaaggtgaccttgaggaaccctgggagctcaggaaggaaggagcgcccagaagcagggacagggagctggttggggaggaccagaaatcaggtgcccaacagcccccttctcctcctttcccttcccttacttccccccttcccctccccttcccctccccctccccaactcagatctggccccggtcccatccccttccctcccccctgccctaagccacctccacctctgtcctggctgcctcagggcgccctgaaaggaccaggacatgcgggtgcggtggctgcgcttttggctcctcttctggctcctgctgggatttatcagccatcagcccacccctgttatcaatactctggctgaccatcgtcatcgtgagactgactttggtggaagtccttggataattatcattattgtgtttctgggacgttacaaatttaccattctcttctgcacaatttacctttgtgtgcctttcctgaagactatcttctggtctcgaaatggacatgatagatccagggatgtacagcagagagctaggaggtccaaccgccgtagacaggaaggaattaaaattggcctggaagacatctgtactttatggaaacaggcggaaacaaaagttcaagctaaaatccgtaagatgaaggtcacaaagaaagtcaaccatcattacaaaatcaatggaaagaggaagaccgccaaagaacaatcaccccctctgcaagaaagcctctttgcaactggctctgtgctcaagccctgcagagggagatggcagagaggaaggctgcctacaagcatcacagtcccatccctgttggtaaccgtgttgtgcaaaaacaccttcatccccacccagtggggcccctgatctaatattcaaagtgtcagaggttccatatttgtaatagcaaatgggccctgactgtaaattagtgaagagtgaatgtaacttattacccacagggacaattccaaatgaaggccttaaatgatgctcagctaagctggttcttgtgtggcctctgtaccttcaaaagctgccgagtcctatgattacacgtgatgggacttgtacacttgaagtgaaacagttttaaaacttgctttgtttagaattcccacctcatttttccatggacaaaagtattctttatgtcctagtgcacttacaatttggtattacctgggagtgaaaagaaatattacagccatgcctaactgacttcttgaggtaagattgttctgtcagaaaaccctctcccagttcccctgcagctcttcaggaatccacatctctgcagagctctttgttctcatgggtggcacctccagagtgaagaagatcctttatcaagaagggaaacaggggaaatgagagggtcctgcaggcagagctggaatcaacttccactctgcctcttgcaagctgtgtgaccccgggcacaatttctccttcctctggaaacctctgttttcttagatttggagcagggtggtcacactgaccttgcagagttccgagaatcagagacagaacataaaaggcctggaaaacattctccaaaaagaagctgcaacatgtgtggacaatgggcttttcatgcctctcttactgtctgttgacctggtgcaagaaacatgctctggtgatggctgtgagggaggaatgaggatagacatagacactcctgtgtctcaaacatgcctctttattactctgttatgactctgtcttccctggggcaggaccccagcctgcctacatttgcagacagacacagtggcatgtggagacaacagtgtgtcccaatgacttttctttactccccagctgtcggcagtactcagtggaagggtgatattatgacactgatactgctattttgaaacctggaggatggaaaggtgcaaaaatctatcaccagcaacagaaggtcagaatggcggcagcggagcatcgtcattcttcaggattgccctactggccctacctcacagctgaaactttaaaaaacaggatgggccgccagccacctcctccaactcaacaacattctataactgataactccctgagcctcaagacacctcccgaatgtcttcttattccccttccaccctctcctcttccaccctccgtggatgataatctcaagactcctcccttagctactcaggaggccgaggcagaaaaatcactgaaacccaagaggcagaggttgagtaagctgagaacaggtcattgcactcaagcctgggcaataagaagaaatctgtgagtggaacaaaagaaaaaaatcaaaaaacaaaacaaaacccacactccaaaaacaaactaacaaagaataaataaataatataaaaataaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 4 - DNA, 665 bp, Artificial Sequence
  lncGRS-4 (RP11-195F19.9)
gggctccgaacgtctcctggggagcggcatgattgaatcagggattccctagccccaggtccccttctccgtcattctcaggacgaggctgcccagttctcagaaaaatgtgaaaagagagactggcctagcctcccagcctacatctttctcccatcctggatgtttcctgcccttgaatgttggactccaagttcttcagttttggaactcggactgtctctccttgctcctcagcctgcagacggcctattgtgggaccttgtgatcgtgttgttgtggtggtattactactactcagcaagattctggttttacttctcatcttgggagttaccatcaagacgttaggtatggctgtgtgacccactttggccaatgaaatgtgaacagaaatggcatatgtcacttgtggacaaaaacatttaagcaagaatttcccttgctgtcttccttgactggagcgaccatggaagcctgtgttgagatgaagaggctgccaacagatcaaaacacttgaaaacttgagcagccacatggactacaactgtgctggagacttgcctggacccacagcaggcttggtatgcatggggaatagaattttgttgtattacactcctcagatttggatgtcatttgttattacagcattacctagccta

SEQ ID NO: 5 - DNA, 1373 bp, Artificial Sequence
  lncGRS-5 (SNHG1)
ctgcctggccagggcgactggcggataaggtcttgtgcgtggcctcgaggcttaaaagtagcagtggggctttgtgaaggacaaaatggcgatggcgggccgtgtaggtcccccttcctatgatgaggaccttttcacagacctgtactgagctccgtgaggataaataactctgaggagatgggccctgcaagcctcttgcttagccgtctgttcagaaaatagcgttttcgaaatgccctgagttgacctaatgtcttattgggctcctgtctgcaggatttacgcgcacgttggaaccgaagagagctctgttgttgcaatgttcagcccacaagagcttactggtgaaggaatgggacaagacccatctttatgcaaagccagcgttacagtaatgttccagcatctcataatctatcctggggaattcagctgcctcccagggtgaatacaggtattcctgatgacagtctgcctctatcttacagagcagcttgttgctatataccattgaaaagccttcagagctgagaggtactactaaccaataacctgcttggctcaaagggccagcaccttctctctaaagcccaagaggagtttgaggaaaactaggtgtctgtgttcactccaggctgaagttacaggtctgagcaaataaggtgtataaaaaatggaatctgtcttggaggacatcagaaggtgaattttccaagttcttggacaacctagctgttgaaaagctttctgggtttggggggtatttcagatgtaccttaaagtgttagcagacacagattaagacactgggagccaatgaaacagcagttgagggtttgctgtgtatcacatttctgtattttatcacccccttcctgcaacattatttatctggaatctacctgcccttttgttttttagatacaagggcttggttttgttacccaggctggtttcaaggccatagctttaagagatcctctcaccacagatttccaaagtgctgggattgcaggtgtgattcatggcacccagactttgctgcctttcttacatgatccaggcccagaacccaaactcaggcactgtatagatgaccactttcgtaaactactgacctagcttgttgccaattgttgattgaacttcccataactccacttcgtgtctgttcctctgtatacagccaccttctgttcccgtcatgagcctttaggtctccatttgcatattgcaaatactatgttccatgtaggtagctcattcagggccttgctcttcacttcaaaaaaggttcccttgaggactggctgtcaatttgtgttgctgtgttggttgttgatgaaaataataaaatgattgattacata

SEQ ID NO: 6 - DNA, 415 bp, Artificial Sequence
  lncGRS-6 (RP1-122P22.2)
cccttccctcccctcctcctctttcctctcctctcccctcccctcccctcctttctcccccttcctcctcttcctcctcctcctcttgctcctcccgggcggggaaacccagccccgcgctcgtctttggggccaccggtcgcccccgcctccgggacctgcggggagggcccgcgagagatggagacgagagatctggacttctgaacatctaccggaaaagataaaatgtgcaagcccttcagaacgctggggaggttgggctgaactgcagtgggagaggagaagagatgctggcgtgaagggctggggcagaagatcaggggaaaattgcggatgatctgaatgatagcagtgaccaccagcaacctacagagctgcccaaccaggaaattgtcagatggaagcaacgctt

SEQ ID NO: 7 - DNA, 1965 bp, Artificial Sequence
  lncGRS-7 (MIR210HG)
gagagggtgccagcggccgcagctgaagttgggccgagagccggcgacggccccgcgccggggtcgcaggcctgcaggagttgagggctgcacctgctcgctggagagggagaggcagatttagtggacgcctggcatggactcggactggcctttggaagctccctgccctgacggggttgcctgtcaccactgcgaagtgaggcttggcaggacctgcacctgagaaaggctgtgtgtggtcttggggtccacacctgcagagctaacttactgccagacggcgacttactgtgggccaccctcagtgaaccggggtgtcctcagctggccctacagagcacttctgtgctggggatgagtaggaactctgggcgaggagggtcccagcgccgcccctcgatacagcctggctctgccctctgcccgtacttacaccaggtgggatccctgccctgcattgcctggggattggctgggcttgggcccgccctgctgtggaactggatgttttcagggagcccagcctttcctcatgtcaacacagttcacaatatagttttcaaagtacagtttaaaactcaaaagtaaacttttcagcaactcaaaggtttgctgagtgatctgaagcactctggccactttttggggccatgggatttggttcacctgaaacagccagtgagaggccgggtgtggtggctcacacccgtaatcccaacacttcaggaggcagacgcgggtgatcgctcacttgagatcaggagttcaagaccagcctgggcaacatggtgaaacctcgtctctactaaaaatacaaaaattagctaggcatggtggtgggcacctgtaatcccagctacttggaaggctgaggcaagagaatcgcttgaacctgggaggtggaggttgcagcgagacgagattacgccgctgcactccagcctgggtgacgagagactctgcctcaaaaaaataaaaaaatgaaacagccagtgaggaggaaggctccccgccttccccccgccggaacatagccatagctgctgctgggacaccctcttggtggggaagaaggctggttagcttcatcagagccagcagcagcagaccagggacgggcacctaggcagtggcctcagagtgaacaggagttcctcagaaacacacacagggacggcgtggcgcatgctctgccagctccatgcctccttcccattgtggggctggggtacgtagggcagagctcatgacctccgggaggacatgggggtgggctctggatggcacctggcattgccccctgctggcctatgtgacggtgtggagggctggtcacagaggtacgaccatccctccagaatgtgggtcggggctgtggatggaggagtaggcccctcatatcccaggcctgctgcccaggcacaacccacttggcctatgcattccaggctccatcccatgtgactctgggcttagccccttctggggccacaggtcaggcaggtccaggccccaaggacctcccagtgacaggcgactgtgagctgggcagacaggagtgaagtcaggtgggggttctggcttgctgacaccagcgtttggagcctcctgctgctgcctggcttccctgcattccctgttccctgcctcaggcaagaaataaccaagccgagttgcctctgcacagcagtgagctcctggtggccctggcttctggggagccctgtggatggcttccttgcccaagtccaggccttcttgttccctttgtgtgctccagagaaagggggcagcaccagatccagatccagggccaaccaacagaaagctgagtccatcccaaactcgcccattctcagagcacaaagaccccatgatctagggcaaacttgtccaactgttggcccatggaacagctttgaatgcagcccaacacaaatctataaattttcttaaacattg

SEQ ID NO: 8 - DNA, 758 bp, Artificial Sequence
  lncGRS-8 (SNHG12)
cccggtgtcgacttactagctgcaagcctctgcctgccttcctgcgcgccgttccccgctagtcgctgctgctggcgcgcactcgccgggtttttcctcccacggcctcgagatggtggtgaatgtggcacggaggagccgggccttccaacccggtgggcccgagctccgaaaggccccctcggcagtgagaggggcgggagcccgcgggggccgcgcccttctctcgcttcggactgcgcaacgctgcgctctgggctgacaggcggataaaacggtcccatcaagactgagaaaaagcacaccagctattggcacagcgtgggcagtggggcctacaggatgactgacttagtctacagagatcccggcgtacttaagcagatgaagactcttaagatgacagaaggtgatttttctggtgatcgaggacttccggggtaatgacagtgatgaaatgcaggggacctggttgcccccaagtttcctggcagtgtgtgatactgaggaggtgagcttgtttctggagctgtgctttaagattcatgttacatgtaaagctgtcctcatttgtgactatggacctatggagttgggacaatctctatgggaagcagaaggcaaggaccccggtcattttaggtagaaacaacagcatgctaatgcaaaaaattatgcagtgtgctactgaacttcagaggtgatcaataaaagaagaataaaaagactaataaaagtaaaaaaaaaaaaaaaaaaaa

SEQ ID NO: 9 - DNA, 409 bp, Artificial Sequence
  lncGRS-9 (RP11-339B21.10)
gggctcagggaccagagcgggattatggaaatcaaaaccaagcttgcagtggaggggagaggtatgggccactttgagaggtgcctcgtgtgggagggggtcagaatggacctcaccattgaggggccgggagaagaggcttctaggcagaagagaacagtgggtgccaaggccctgaggcaggaaggtgtttaggagcagaaagaaggaagcctggggcttagcatgtgctcagtacttggccaagaagcctcggcccaggggctgggagtgtgagcgcaggaaagaggttgctggacacggcaggagagaatgggaactgggccctccaagggcagcccttccaggagatgggacatggattccttgtttgtgttctctgctatagccaaatatatgtgcacgtgta

SEQ ID NO: 10 - DNA, 16 bp, Artificial Sequence
  ASO targeting sequence CTC-338M12.4 ASO 1
ccaacgggaaaggcgg

SEQ ID NO: 11 - DNA, 16 bp, Artificial Sequence
  ASO targeting sequence CTC-338M12.4 ASO 2
cagaagaacggtggac

SEQ ID NO: 12 - protein, 1659 aa, Artificial Sequence
  Synthetic dCas9-p65AD fusion protein
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEGAPKKKRKVGSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSGS

SEQ ID NO: 13 - protein, 1453 aa, Artificial Sequence
  Synthetic dCAS9-VP64 fusion protein
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEGAPKKKRKVGSSGSPKKKRKVGSDALDDFDLDMLGSDALDDFDLDMLGSDALDDFDLDMLGSDALDDFDLDMLGSPKKKRKVGS

SEQ ID NO: 14 - protein, 1492 aa, Artificial Sequence
  Synthetic dCas9-KRAB fusion protein
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGIGSGSNGSSGSGGSGGGSMDAKSLTAWSRTLVTFKDVFVDFTREEWKLLDTAQQIVYRNVMLENYKNLVSLGYQLTKPDVILRLEKGEEP

SEQ ID NO: 15 - protein, 1515 aa, Artificial Sequence
  Synthetic dCas9-CSD fusion protein
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGIGSGSNGSSGSGGSGGGSSADDIKSKKKREQSNDIARGFERGLEPEKIIGATDSCGDLMFLMKWKDTDEADLVLAKEANVKCPQIVIAFYEERLTWHAYPEDAENKEKETAKS

SEQ ID NO: 16 - DNA, 16 bp, Artificial Sequence
  ASO targeting sequence TP53 ASO
ccaatgcgcctgatta

SEQ ID NO: 17 - DNA, 16 bp, Artificial Sequence
  ASO targeting sequence POLA1 ASO
catttagtgtaagcgg

SEQ ID NO: 18 - DNA, 20 bp, Artificial Sequence
  sgRNA protospacer sequence CTC-338M12.4 sgRNA 1
gaaagagggcggccccggag

SEQ ID NO: 19 - DNA, 20 bp, Artificial Sequence
  sgRNA protospacer sequence CTC-338M12.4 sgRNA 2
ggaaccgggccgggagacgg

SEQ ID NO: 20 - DNA, 20 bp, Artificial Sequence
  sgRNA protospacer sequence ERCC6L2 sgRNA 1
ggcgcggaaacctcaggcaa

SEQ ID NO: 21 - DNA, 20 bp, Artificial Sequence
  sgRNA protospacer sequence ERCC6L2 sgRNA 2
ggcgctcgccccttacgcag

SEQ ID NO: 22 - DNA, 18 bp, Artificial Sequence
  qPCR primer
ttcattgtgggagcagac

SEQ ID NO: 23 - DNA, 18 bp, Artificial Sequence
  qPCR primer
cagcagtttctccagagc

SEQ ID NO: 24 - DNA, 20 bp, Artificial Sequence
  qPCR primer
atgcgagttgttctccgtct

SEQ ID NO: 25 - DNA, 20 bp, Artificial Sequence
  qPCR primer
tatctgcggtttcctcaagc

SEQ ID NO: 26 - DNA, 20 bp, Artificial Sequence
  qPCR primer
gtggctgttggagtcggtat

SEQ ID NO: 27 - DNA, 20 bp, Artificial Sequence
  qPCR primer
attcactccccaccctctct

SEQ ID NO: 28 - DNA, 19 bp, Artificial Sequence
  qPCR primer
acgaaggtggttttcccag

SEQ ID NO: 29 - DNA, 18 bp, Artificial Sequence
  qPCR primer
gtcccccactaccacaaa

SEQ ID NO: 30 - DNA, 20 bp, Artificial Sequence
  qPCR primer
gggaaattcaacggcacagt

SEQ ID NO: 31 - DNA, 19 bp, Artificial Sequence
  qPCR primer
agatggtgatgggcttccc

SEQ ID NO: 32 - DNA, 20 bp, Artificial Sequence
  qPCR primer
ctacctgccttttggggaag

SEQ ID NO: 33 - DNA, 20 bp, Artificial Sequence
  qPCR primer
ttcgtcttgactctgcaagc

SEQ ID NO: 34 - DNA, 19 bp, Artificial Sequence
  qPCR primer
gctatgtggaagatgccga

SEQ ID NO: 35 - DNA, 20 bp, Artificial Sequence
  qPCR primer
tgttcggttttgtcactgcg

SEQ ID NO: 36 - DNA, 20 bp, Artificial Sequence
  qPCR primer
gcccaacaacaccagctcct

SEQ ID NO: 37 - DNA, 20 bp, Artificial Sequence
  qPCR primer
cctgggcatccttgagttcc

SEQ ID NO: 38 - DNA, 886 bp, Artificial Sequence
  Complementary sequence to lncGRS-1 (CTC-338M12.4)
tatttttgtataatggtttatttaatacagtatacattatctttcatatactttgttacaatgttaaaaaaacttcatttttcataatataggaaataaagttggaaacaactctggtgggaatacttcagatagagcaagaaagcattcacagcaaggcctataatcagtaagatgtgtgaaaagttgggtagccacaggaggtgttcattaaggatatgattccatttatatagctatttctattgcataaccaggacagttttattgttttgaggtcaatgttcttttaaaatttgattttctgtaagaagaggctttttggcccagaaagccttacttattttacatcttccagtttgtccatcccatgagttagagttcgtcttgactctgcaagcttgtatggaatggaagtggtgcctctggggtcccagacacgggagagaggaagcagcccaggggctgggagaagtggtgagacccagaagaacggtggacccagtcaggaagggtcctgccagcagggcgtgggccagcctcggggtcttccccaaaaggcaggtaggggcctccagcaagcttcccacctccgtgaggctggagaagaggccccctggatgagaaggaagaggccggcaggtccggcgggcgcaggcacaggtagacttctggtgggaaaagcgtgactcagcctcctccagcggctgcaggacgtccccccgctagaaagggccgggctccctttgctgacccagagtcaacccagcgattcctcccggggaggcggggggccccggccccggcccagagcgcatgcctatgccgggagctgggcgaggacagggcacagccacagctgggtgcccaacgggaaaggcggccg

SEQ ID NO: 39 - DNA, 371 bp, Artificial Sequence
  Complementary sequence to lncGRS-2 (AC005624.2)
cccagccaaaaaaatttttaataattagccacatgtgctggcacacacctgtagtcccagctcctcgggaggctgaggcaggagaatggcttgcatccaggagttcaagtctgcagtcagccatgattgcgccactgcactccagtgtgggcaacggagcgagaccccccatacccagcctttgcccgtgctgttccatccgccaggagcgccgtttcctgcctgactgcgcggccctgggcctcagtcttgcgctctgcaaaccggggtctcgaatctaccccagccctcctgtctcggcgctggggaggggcttcgggtgcccagagccatccccgcgggacccccagggactggactccaagtcccag

SEQ ID NO: 40 - DNA, 4298 bp, Artificial Sequence
  Complementary sequence to lncGRS-3 (PDXDC2P)
ttttttttttttttttttttttttttttttttttttttttttttttttttttttttttttttttttttttttttttttttttttttttttttttttttttttttttttttttttttttatttttatattatttatttattctttgttagtttgtttttggagtgtgggttttgttttgttttttgatttttttcttttgttccactcacagatttcttcttattgcccaggcttgagtgcaatgacctgttctcagcttactcaacctctgcctcttgggtttcagtgatttttctgcctcggcctcctgagtagctaagggaggagtcttgagattatcatccacggagggtggaagaggagagggtggaaggggaataagaagacattcgggaggtgtcttgaggctcagggagttatcagttatagaatgttgttgagttggaggaggtggctggcggcccatcctgttttttaaagtttcagctgtgaggtagggccagtagggcaatcctgaagaatgacgatgctccgctgccgccattctgaccttctgttgctggtgatagatttttgcacctttccatcctccaggtttcaaaatagcagtatcagtgtcataatatcacccttccactgagtactgccgacagctggggagtaaagaaaagtcattgggacacactgttgtctccacatgccactgtgtctgtctgcaaatgtaggcaggctggggtcctgccccagggaagacagagtcataacagagtaataaagaggcatgtttgagacacaggagtgtctatgtctatcctcattcctccctcacagccatcaccagagcatgtttcttgcaccaggtcaacagacagtaagagaggcatgaaaagcccattgtccacacatgttgcagcttctttttggagaatgttttccaggccttttatgttctgtctctgattctcggaactctgcaaggtcagtgtgaccaccctgctccaaatctaagaaaacagaggtttccagaggaaggagaaattgtgcccggggtcacacagcttgcaagaggcagagtggaagttgattccagctctgcctgcaggaccctctcatttcccctgtttcccttcttgataaaggatcttcttcactctggaggtgccacccatgagaacaaagagctctgcagagatgtggattcctgaagagctgcaggggaactgggagagggttttctgacagaacaatcttacctcaagaagtcagttaggcatggctgtaatatttcttttcactcccaggtaataccaaattgtaagtgcactaggacataaagaatacttttgtccatggaaaaatgaggtgggaattctaaacaaagcaagttttaaaactgtttcacttcaagtgtacaagtcccatcacgtgtaatcataggactcggcagcttttgaaggtacagaggccacacaagaaccagcttagctgagcatcatttaaggccttcatttggaattgtccctgtgggtaataagttacattcactcttcactaatttacagtcagggcccatttgctattacaaatatggaacctctgacactttgaatattagatcaggggccccactgggtggggatgaaggtgtttttgcacaacacggttaccaacagggatgggactgtgatgcttgtaggcagccttcctctctgccatctccctctgcagggcttgagcacagagccagttgcaaagaggctttcttgcagagggggtgattgttctttggcggtcttcctctttccattgattttgtaatgatggttgactttctttgtgaccttcatcttacggattttagcttgaacttttgtttccgcctgtttccataaagtacagatgtcttccaggccaattttaattccttcctgtctacggcggttggacctcctagctctctgctgtacatccctggatctatcatgtccatttcgagaccagaagatagtcttcaggaaaggcacacaaaggtaaattgtgcagaagagaatggtaaatttgtaacgtcccagaaacacaataatgataattatccaaggacttccaccaaagtcagtctcacgatgacgatggtcagccagagtattgataacaggggtgggctgatggctgataaatcccagcaggagccagaagaggagccaaaagcgcagccaccgcacccgcatgtcctggtcctttcagggcgccctgaggcagccaggacagaggtggaggtggcttagggcaggggggagggaaggggatgggaccggggccagatctgagttggggagggggaggggaaggggaggggaaggggggaagtaagggaagggaaaggaggagaagggggctgttgggcacctgatttctggtcctccccaaccagctccctgtccctgcttctgggcgctccttccttcctgagctcccagggttcctcaaggtcaccttcctcaggtactcgcatgggcatgtcactgactttacgtgctgctgcagctccttggtgagcgggccctggtcatgggacaggaactgtggggtcaggacgatagagagcttcaccatttgcagaatgagaacaggggctcatcatgagtgccaacctcagttttcttctgtgtgaaacaggaacaatagtttcattcctcattcagtttctctcaaggtttcacgagatcatacctgctgcggtcatcaaagggctgaaccgcaaacacgtgccctcagcttccagatccatgactgtgaggccgctcgcaggcaccagctgcttcagctgttctcccagccagagattcagcgcatcacacgagtgcctctcccggccgactgctgaaggtgtcatgttgggcactgggacggctttaaacactggatctgagcctggtaattcttggaaaaatctgaacaccaccactggggagctgagctcatcttccaccaagattttgatgtaattcactttcttcaaactttcctgcaaccattgactcagttgacaggcatgcttgatcctctccacaaacccatcaagtcccaagtattgtaaagataaccacagaggcagggcacggagtttgtctgtgggcttatttgatataagaccagcaactaaagtcaaggcagggtcgtgtttatacagtgtcaccgcaggaacagctggcaaacccagccacgggccaggagtcatcgtcatgctatcacatttggctgcagccagcactgatgaggagacataacccagagccaatgttgccagattcacaccctccacatgaagccatatgccatactgctcacagagttctttcaatctcccaatcttgtctgtgtgtcctactgctgccgttcctgcatttgcgacaagcaacaggggcagtcttcctcgctctatatcatctttaatcagtttctccaggaaggcaacatccatctgatgctgggatccaaacatagtgttacagggtacacggcacaagcaggggaagggcaagccgagctgattacaaaggtattggcccaggccaggtctagcagcagcactaagatatatgacaggcttcttgttatataacccactgaagccatccactacgaagtcttcatattgagaatgaatggcaagcctacatatctttgcaagtccttctctttcctcttcgtggaaataagcacacccattttcatatctgaaaattctgcatagccataaggtggtatctgaaagtatcctagttgtaagttttctcagcttctcttcgtccagagtcaaaatataagctcccagactatgtcccaacacagccacatgaccttgttctccaatattttggattctggggctctggggctcctcatcttcatctccatgcatcagattctgaactaactggaggatgctcaccatatcttgtccactgccctggagtgggcctggaatatctctggatatgagcttctttccattttcctcttctgttcttctctgactgtcctccagcatcttcactgcctccttcaagttttttcccatttcagctaacgtggggtctgctatcttctccagggacgcgtccatggctgaggcggccggtggtggcccggcggtcccgggctctacttcctcctcccgcgccccgcagctctttccacgctggcagcgctgacgtcccccgcgcccgcggccagcggggcggagaagaggaggagctgccaggcggcgccgccggctgccgaacctgatggttgag

SEQ ID NO: 41 - DNA, 665 bp, Artificial Sequence
  Complementary sequence to lncGRS-4 (RP11-195F19.9)
taggctaggtaatgctgtaataacaaatgacatccaaatctgaggagtgtaatacaacaaaattctattccccatgcataccaagcctgctgtgggtccaggcaagtctccagcacagttgtagtccatgtggctgctcaagttttcaagtgttttgatctgttggcagcctcttcatctcaacacaggcttccatggtcgctccagtcaaggaagacagcaagggaaattcttgcttaaatgtttttgtccacaagtgacatatgccatttctgttcacatttcattggccaaagtgggtcacacagccatacctaacgtcttgatggtaactcccaagatgagaagtaaaaccagaatcttgctgagtagtagtaataccaccacaacaacacgatcacaaggtcccacaataggccgtctgcaggctgaggagcaaggagagacagtccgagttccaaaactgaagaacttggagtccaacattcaagggcaggaaacatccaggatgggagaaagatgtaggctgggaggctaggccagtctctcttttcacatttttctgagaactgggcagcctcgtcctgagaatgacggagaaggggacctggggctagggaatccctgattcaatcatgccgctccccaggagacgttcggagccc

SEQ ID NO: 42 - DNA, 1372 bp, Artificial Sequence
  Complementary sequence to lncGRS-5 (SNHG1)
tatgtaatcaatcattttattattttcatcaacaaccaacacagcaacacaaattgacagccagtcctcaagggaaccttttttgaagtgaagagcaaggccctgaatgagctacctacatggaacatagtatttgcaatatgcaaatggagacctaaaggctcatgacgggaacagaaggtggctgtatacagaggaacagacacgaagtggagttatgggaagttcaatcaacaattggcaacaagctaggtcagtagtttacgaaagtggtcatctatacagtgcctgagtttgggttctgggcctggatcatgtaagaaaggcagcaaagtctgggtgccatgaatcacacctgcaatcccagcactttggaaatctgtggtgagaggatctcttaaagctatggccttgaaaccagcctgggtaacaaaaccaagcccttgtatctaaaaaacaaaagggcaggtagattccagaaaataatgttgcaggaagggggtgataaaatacagaaatgtgatacacagcaaaccctcaactgctgtttcattggctcccagtgtcttaatctgtgtctgctaacactttaaggtacatctgaaataccccccaaacccagaaagcttttcaacagctaggttgtccaagaacttggaaaattcaccttctgatgtcctccaagacagattccattttttatacaccttatttgctcagacctgtaacttcagcctggagtgaacacagacacctagttttcctcaaactcctcttgggctttagagagaaggtgctggccctttgagccaagcaggttattggttagtagtacctctcagctctgaaggcttttcaatggtatatagcaacaagctgctctgtaagatagaggcagactgtcatcaggaatacctgtattcaccctgggaggcagctgaattccccaggatagattatgagatgctggaacattactgtaacgctggctttgcataaagatgggtcttgtcccattccttcaccagtaagctcttgtgggctgaacattgcaacaacagagctctcttcggttccaacgtgcgcgtaaatcctgcagacaggagcccaataagacattaggtcaactcagggcatttcgaaaacgctattttctgaacagacggctaagcaagaggcttgcagggcccatctcctcagagttatttatcctcacggagctcagtacaggtctgtgaaaaggtcctcatcataggaagggggacctacacggcccgccatcgccattttgtccttcacaaagccccactgctacttttaagcctcgaggccacgcacaagaccttatccgccagtcgccctggccaggcag

SEQ ID NO: 43 - DNA, 415 bp, Artificial Sequence
  Complementary sequence to lncGRS-6 (RP1-122P22.2)
aagcgttgcttccatctgacaatttcctggttgggcagctctgtaggttgctggtggtcactgctatcattcagatcatccgcaattttcccctgatcttctgccccagcccttcacgccagcatctcttctcctctcccactgcagttcagcccaacctccccagcgttctgaagggcttgcacattttatcttttccggtagatgttcagaagtccagatctctcgtctccatctctcgcgggccctccccgcaggtcccggaggcgggggcgaccggtggccccaaagacgagcgcggggctgggtttccccgcccgggaggagcaagaggaggaggaggaagaggaggaagggggagaaaggaggggaggggaggggagaggagaggaaagaggaggaggggagggaaggg

SEQ ID NO: 44 - DNA, 1965 bp, Artificial Sequence
  Complementary sequence to lncGRS-7 (MIR210HG)
caatgtttaagaaaatttatagatttgtgttgggctgcattcaaagctgttccatgggccaacagttggacaagtttgccctagatcatggggtctttgtgctctgagaatgggcgagtttgggatggactcagctttctgttggttggccctggatctggatctggtgctgccccctttctctggagcacacaaagggaacaagaaggcctggacttgggcaaggaagccatccacagggctccccagaagccagggccaccaggagctcactgctgtgcagaggcaactcggcttggttatttcttgcctgaggcagggaacagggaatgcagggaagccaggcagcagcaggaggctccaaacgctggtgtcagcaagccagaacccccacctgacttcactcctgtctgcccagctcacagtcgcctgtcactgggaggtccttggggcctggacctgcctgacctgtggccccagaaggggctaagcccagagtcacatgggatggagcctggaatgcataggccaagtgggttgtgcctgggcagcaggcctgggatatgaggggcctactcctccatccacagccccgacccacattctggagggatggtcgtacctctgtgaccagccctccacaccgtcacataggccagcagggggcaatgccaggtgccatccagagcccacccccatgtcctcccggaggtcatgagctctgccctacgtaccccagccccacaatgggaaggaggcatggagctggcagagcatgcgccacgccgtccctgtgtgtgtttctgaggaactcctgttcactctgaggccactgcctaggtgcccgtccctggtctgctgctgctggctctgatgaagctaaccagccttcttccccaccaagagggtgtcccagcagcagctatggctatgttccggcggggggaaggcggggagccttcctcctcactggctgtttcatttttttatttttttgaggcagagtctctcgtcacccaggctggagtgcagcggcgtaatctcgtctcgctgcaacctccacctcccaggttcaagcgattctcttgcctcagccttccaagtagctgggattacaggtgcccaccaccatgcctagctaatttttgtatttttagtagagacgaggtttcaccatgttgcccaggctggtcttgaactcctgatctcaagtgagcgatcacccgcgtctgcctcctgaagtgttgggattacgggtgtgagccaccacacccggcctctcactggctgtttcaggtgaaccaaatcccatggccccaaaaagtggccagagtgcttcagatcactcagcaaacctttgagttgctgaaaagtttacttttgagttttaaactgtactttgaaaactatattgtgaactgtgttgacatgaggaaaggctgggctccctgaaaacatccagttccacagcagggcgggcccaagcccagccaatccccaggcaatgcagggcagggatcccacctggtgtaagtacgggcagagggcagagccaggctgtatcgaggggcggcgctgggaccctcctcgcccagagttcctactcatccccagcacagaagtgctctgtagggccagctgaggacaccccggttcactgagggtggcccacagtaagtcgccgtctggcagtaagttagctctgcaggtgtggaccccaagaccacacacagcctttctcaggtgcaggtcctgccaagcctcacttcgcagtggtgacaggcaaccccgtcagggcagggagcttccaaaggccagtccgagtccatgccaggcgtccactaaatctgcctctccctctccagcgagcaggtgcagccctcaactcctgcaggcctgcgaccccggcgcggggccgtcgccggctctcggcccaacttcagctgcggccgctggcaccctctc

SEQ ID NO: 45 - DNA, 758 bp, Artificial Sequence
  Complementary sequence to lncGRS-8 (SNHG12)
ttttttttttttttttttttacttttattagtctttttattcttcttttattgatcacctctgaagttcagtagcacactgcataattttttgcattagcatgctgttgtttctacctaaaatgaccggggtccttgccttctgcttcccatagagattgtcccaactccataggtccatagtcacaaatgaggacagctttacatgtaacatgaatcttaaagcacagctccagaaacaagctcacctcctcagtatcacacactgccaggaaacttgggggcaaccaggtcccctgcatttcatcactgtcattaccccggaagtcctcgatcaccagaaaaatcaccttctgtcatcttaagagtcttcatctgcttaagtacgccgggatctctgtagactaagtcagtcatcctgtaggccccactgcccacgctgtgccaatagctggtgtgctttttctcagtcttgatgggaccgttttatccgcctgtcagcccagagcgcagcgttgcgcagtccgaagcgagagaagggcgcggcccccgcgggctcccgcccctctcactgccgagggggcctttcggagctcgggcccaccgggttggaaggcccggctcctccgtgccacattcaccaccatctcgaggccgtgggaggaaaaacccggcgagtgcgcgccagcagcagcgactagcggggaacggcgcgcaggaaggcaggcagaggcttgcagctagtaagtcgacaccggg

SEQ ID NO: 46 - DNA, 409 bp, Artificial Sequence
  Complementary sequence to lncGRS-9 (RP11-339B21.10)
tacacgtgcacatatatttggctatagcagagaacacaaacaaggaatccatgtcccatctcctggaagggctgcccttggagggcccagttcccattctctcctgccgtgtccagcaacctctttcctgcgctcacactcccagcccctgggccgaggcttcttggccaagtactgagcacatgctaagccccaggcttccttctttctgctcctaaacaccttcctgcctcagggccttggcacccactgttctcttctgcctagaagcctcttctcccggcccctcaatggtgaggtccattctgaccccctcccacacgaggcacctctcaaagtggcccatacctctcccctccactgcaagcttggttttgatttccataatcccgctctggtccctgagccc

SEQ ID NO: 47 - DNA, 20 bp, Artificial Sequence
  sgRNA protospacer sequence AC005624.2 sgRNA 1
gcatccccgcgggaccccca

SEQ ID NO: 48 - DNA, 20 bp, Artificial Sequence
  sgRNA protospacer sequence AC005624.2 sgRNA 2
gctgtctcggcgctggggag

SEQ ID NO: 49 - DNA, 20 bp, Artificial Sequence
  sgRNA protospacer sequence PDXDC2P sgRNA 1
ggcggggcggggccctgtaa

SEQ ID NO: 50 - DNA, 20 bp, Artificial Sequence
  sgRNA protospacer sequence PDXDC2P sgRNA 2
ggttccgtgggggccgcgag

SEQ ID NO: 51 - DNA, 20 bp, Artificial Sequence
  sgRNA protospacer sequence RP11-195F19.9 sgRNA 1
gtgagtggaggcgggttctg

SEQ ID NO: 52 - DNA, 20 bp, Artificial Sequence
  sgRNA protospacer sequence RP11-195F19.9 sgRNA 2
ggttctgcggaggaggaacc

SEQ ID NO: 53 - DNA, 20 bp, Artificial Sequence
  sgRNA protospacer sequence SNHG1 sgRNA 1
gatgagaacgaatctccccg

SEQ ID NO: 54 - DNA, 20 bp, Artificial Sequence
  sgRNA protospacer sequence SNHG1 sgRNA 2
gaagacattaggtcaactca

SEQ ID NO: 55 - DNA, 20 bp, Artificial Sequence
  sgRNA protospacer sequence RP1-122P22.2 sgRNA 1
ggcccgcgagaggtaagggg

SEQ ID NO: 56 - DNA, 20 bp, Artificial Sequence
  sgRNA protospacer sequence RP1-122P22.2 sgRNA 2
gccgcaggtcccggaggcgg

SEQ ID NO: 57 - DNA, 20 bp, Artificial Sequence
  sgRNA protospacer sequence MIR210HG sgRNA 1
gctgaagttgggccgagagc

SEQ ID NO: 58 - DNA, 20 bp, Artificial Sequence
  sgRNA protospacer sequence MIR210HG sgRNA 2
ggtcgggccggggggcgaga

SEQ ID NO: 59 - DNA, 20 bp, Artificial Sequence
  sgRNA protospacer sequence SNHG12 sgRNA 1
gtaagtcgacaccgggaatg

SEQ ID NO: 60 - DNA, 20 bp, Artificial Sequence
  sgRNA protospacer sequence SNHG12 sgRNA 2
ggccccctcggcagtgagag

SEQ ID NO: 61 - DNA, 20 bp, Artificial Sequence
  sgRNA protospacer sequence RP11-339B21.10 sgRNA 1
gcagtggaggggagaggtat

SEQ ID NO: 62 - DNA, 20 bp, Artificial Sequence
  sgRNA protospacer sequence RP11-339B21.10 sgRNA 2
gtcttctcccggcccctcaa

SEQ ID NO: 63 - DNA, 93 bp, Artificial Sequence
  sgRNA
gtttaagagctaagctggaaacagcatagcaagtttaaataaggctagtccgttatcaacttgaaaaagtggcaccgagtcggtgcttttttt

(19) United States
(12) Patent Application Publication
(10) Pub. No.: US 2023/0193250 A1
(43) Pub. Date: Jun. 22, 2023

(54) COMPOSITIONS AND METHODS OF TREATING GLIOMA

(71) Applicant: THE REGENTS OF THE UNIVERSITY OF CALIFORNIA, OAKLAND, CA (US)

(72) Inventors: Daniel A. Lim, San Francisco, CA (US); Martina Malatesta, San Francisco, CA (US); Siyuan John Liu, San Francisco, CA (US)

(21) Appl. No.: 17/911,633

(22) PCT Filed: Mar. 15, 2021
(86) PCT No.: PCT/US21/22442

§ 371 Date: Sep. 14, 2022

Related U.S. Application Data
(60) Provisional application No. 62/989,652, filed on Mar. 14, 2020.

(51) Int. Cl. C12N 15/10

(52) U.S. Cl. CPC C12N 15/1079; C12N 15/102; C12N 15/1082

(57) ABSTRACT

The present disclosure relates generally to detection of long noncoding RNA (lncRNA) molecules in a sample or diagnosis of subject based upon detection of long noncoding RNAs in a sample, specifically to identify and use of molecular biomarkers or cancer including malignant glioma. Also provided are compositions and methods for treating malignant glioma in a subject, as well as human brain organoid models of malignant glioma and methods of generating and using the same.

### CROSS-REFERENCE TO RELATED APPLICATIONS

This application claims priority to U.S. Provisional Application No. 62/989,652 filed on Mar. 14, 2020, which is incorporated by reference herein in its entirety.

### GOVERNMENT SUPPORT

This invention was made with government support under grants Nos. P30 EY002162, R01 NS091544, R03 AG063157, and R21 NS101395 awarded by The National Institutes of Health. The government has certain rights in the invention.

### TECHNOLOGY FIELD

The present disclosure relates generally to detection of long noncoding RNA (lncRNA) molecules in a sample or diagnosis of subject based upon detection of long noncoding RNAs in a sample, specifically to identify and use of molecular biomarkers for cancer including malignant glioma. Also provided are compositions and methods for treating malignant glioma in a subject, as well as human brain organoid models of malignant glioma and methods of generating and using the same.

### BACKGROUND

The human genome produces many thousands of lncRNAs [1-3]—transcripts longer than 200 nucleotides that do not encode for proteins—and certain lncRNAs play key roles in the pathogenesis of cancer [4-7]. LncRNAs exhibit highly cell type-specific expression and function [1,8], making this class of transcripts attractive for targeted cancer therapy. However, it is currently not possible to predict which of these non-coding transcripts would be effective therapeutic targets, let alone those that could sensitize cancer cells to radiation. To rapidly develop lncRNAs as a class of targets for cancer therapy, systematic functional screens are necessary.

CRISPR-based technologies have enabled genome-scale screens of gene function in mammalian cells [8-16]. These screening methods have been valuable to the identification of genes—non-coding in addition to coding—that are essential for various cellular phenotypes. However, whether such screen-identified hits can increase the efficacy of ionizing radiation—a critical adjunctive cancer therapy for many malignancies—has not been systematically studied at large scale.

Malignant glioma—a primary cancer of the central nervous system (CNS)—is a fatal diagnosis for most patients [17]. Despite surgery and adjuvant therapy such as fractionated radiation, adults with glioblastoma (GBM) have a median survival of only 14 months [18,19]. In children, the most common malignant glioma is diffuse intrinsic pontine glioma (DIPG). DIPG is primarily treated with radiotherapy, but median survival is only 9-10 months, and few patients survive more than two years after diagnosis [20-22]. While radiation is a critical component of the treatment of both adult and pediatric malignant gliomas by reducing tumor growth [19,23,24], the toxicity of radiation to normal brain cells limits the total dose that can be delivered, and glioma cells that survive radiation lead to tumor recurrence. Thus, therapeutics that increase the efficacy of radiation without reducing the viability of normal brain cells would be of critical clinical benefit, complementing the current standard-of-care for nearly all patients with malignant gliomas.

Organoids are miniature three dimensional (3D) representations of their in vivo counterpart organs, and organoid-based models of cancer are emerging as a useful platform for the evaluation of therapeutics [25]. Human brain organoids have been generated from pluripotent stem cell (PSC) populations, mimicking early stages of fetal brain development [26-28]. Such embryonic brain organoids have been useful to study the genetic mutations that cause GBM [29] and can also serve as a 3D tissue substrate for the growth of glial tumors [30]. However, embryonic brain organoids do not closely represent the mature brain tissue of glioma patients, limiting their utility for assessing potential drug toxicity to normal adult brain cells.

### SUMMARY

A radiation modifier screen using CRISPRi was developed to identify specific lncRNAs that sensitize glioma cells to radiotherapy. In this screen of 5689 lncRNA loci, 467 hits were found to modify cell growth in the presence of radiation. 33 of these hits sensitized cells to clinically relevant doses of fractionated radiation, and based on their expression in adult and pediatric glioma, nine of these hits were prioritized as lncRNA Glioma Radiation Sensitizers (lncGRS). Knockdown of lncGRS-1 (CTC-338M12.4 located on chromosome 5 q35.3) inhibited the growth of primary adult and pediatric glioma cells, but the viability of normal brain cells was not harmed. While lncGRS-1 is primate-conserved, this lncRNA does not exist in rodents, making traditional in vivo mouse models of glioma suboptimal for assessing potential toxicity of lncGRS-1 knockdown in normal brain tissue. A novel human brain organoid model of malignant glioma was therefore developed. To mimic the brains of patients, “mature” human brain organoids (MBOs) were assembled from mature neural cells types derived from induced pluripotent stem cells (iPSCs), and human glioma cells grew invasively within these 3D tissues. Furthermore, antisense oligonucleotides (ASOs) targeting lncGRS-1 selectively decreased tumor growth and sensitized glioma cells to radiation therapy. These studies identify lncGRS-1 as a glioma-specific therapeutic target and establish a generalizable approach to rapidly identify novel therapeutic targets in the vast non-coding genome.

Described herein is the identification of certain lncRNAs as glioma-specific therapeutic targets using a generalizable approach to rapidly identify therapeutic targets in the vast non-coding genome to enhance the standard-of-care for cancer therapy, particularly glioma therapy. Also disclosed are compositions and methods of diagnosing and/or treating malignant glioma, as well as method of inhibiting growth and/or proliferation of glioma cells. A method for generation of a human brain organoid model of malignant glioma and the use for screening an agent capable of synergizing with radiation to reduce growth of glioma cells is also provided.

In one aspect, the present disclosure relates to a method of identifying a subject that is a radiotherapy sensitizer comprising: a) exposing the plurality of test cells to a radiation; b) selecting a test cell having decreased cell proliferation as compared to a control cell treated with the radiation alone; and c) identifying the locus targeted by an sgRNA in the test cell selected in step (b) as the radiotherapy sensitizer; wherein the plurality of test cells each comprise: i) the small guide RNA (sgRNA) that targets a locus encoding a long non-coding RNA; and ii) a nuclease-deficient sgRNA-mediated nuclease (dCas9), wherein the dCas9 comprises a dCas9 domain fused to a transcriptional modulator. In some embodiments, the method of the present disclosure further comprises a step of obtaining the plurality of test cells for screening.

In some embodiments, the cells are glioblastomas (GBM) cells. In some embodiments, the transcriptional modulator fused to the dCas9 domain is a transcriptional activator or a transcriptional repressor. In some embodiments, the dCas9 comprises an amino acid sequence having at least about 70% sequence identity to the amino acid sequence of SEQ ID NO: 12, SEQ ID NO: 13, SEQ ID NO: 14 or SEQ ID NO: 15. In some embodiments, the cells are exposed at a total dose of from about 1 Gy to about 12 Gy and delivered in about 1 to about 6 fractions. In some embodiments, the radiation used to expose the cells is at a total dose of about 8 Gy and delivered in about 4 fractions. In some embodiments, the locus targeted by CRISPRi comprises a nucleic acid sequence having at least about 70% sequence identity to any of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 and SEQ ID NO: 9.

In another aspect, the present disclosure relates to a method of diagnosing malignant glioma in a subject comprising: a) exposing a sample from the subject to a nucleic acid complementary to a long noncoding RNA (lncRNA) comprising a nucleic acid sequence having at least about 70% sequence identity to any onr or plurality of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 and SEQ ID NO: 9; and b) detecting binding between an lncRNA in the sample and the nucleic acid complementary to the lncRNA; wherein binding between the lncRNA and the nucleic acid complementary to the lncRNA is indicative of said subject having malignant glioma.

In some embodiments, the method further comprises obtaining a sample from the subject. In some embodiments, the sample is a human tissue sample, serum, plasma, blood draw, brushing, biopsy, or surgical resection of the subject. In some embodiments, the interaction between the sample and the nucleic acid complementary to the lncRNA is detected by fluorescence, radioactivity, enzyme-linked electrochemical, or chemoluminescence. In some embodiments, the lncRNA comprises a nucleic acid sequence having at least about 70% sequence identity to SEQ ID NO: 1. In some embodiments, the lncRNA comprises the nucleic acid sequence of SEQ ID NO: 1.

In some embodiments, the method further comprises a step of providing the subject a treatment regime to manage the malignant glioma. In some embodiments, the treatment regime to manage the malignant glioma comprises a radiotherapy. In some embodiments, the malignant glioma is glioblastoma or diffuse intrinsic pontine gliomas.

In a further aspect, the present disclosure relates to a method of treating malignant glioma in a subject in need thereof comprising administering to the subject a therapeutically effective amount of an agent that knockdowns expression of a long noncoding RNA (lncRNA) having at least about 70% sequence identity to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 and SEQ ID NO: 9. In some embodiments, the agent that knockdowns expression of the lncRNA is a microRNA (miRNA), small interfering RNA (siRNA), antisense oligonucleotide (ASO), or morpholino. In some embodiments, the lncRNA comprises a nucleic acid sequence having at least about 70% sequence identity to SEQ ID NO: 1. In some embodiments, the lncRNA comprises the nucleic acid sequence of SEQ ID NO: 1. In some embodiments, the agent that knockdowns expression of the lncRNA is an ASO comprising a nucleic acid sequence having at least about 70% sequence identity to SEQ ID NO: 10 or SEQ ID NO: 11. In some embodiments, the subject is concurrently under radiation therapy. In some embodiments, the agent that knockdowns expression of the lncRNA increases the effect of the radiation therapy compared to administering the radiation therapy alone. In some embodiments, the malignant glioma being treated is glioblastoma or diffuse intrinsic pontine gliomas.

In another aspect, the present disclosure relates to a method of inhibiting growth and/or proliferation of glioma cells in a subject in need thereof comprising administering to the subject a therapeutically effective amount of an agent that knockdowns expression of a long noncoding RNA (lncRNA) having at least about 70% sequence identity to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 AND SEQ ID NO: 9. In some embodiments, the agent that knockdowns expression of the lncRNA is a miRNA, siRNA, antisense oligonucleotide (ASO), or morpholino. In some embodiments, the lncRNA comprises a nucleic acid sequence having at least about 70% sequence identity to SEQ ID NO: 1. In some embodiments, the lncRNA comprises the nucleic acid sequence of SEQ ID NO: 1. In some embodiments, the agent that knockdowns expression of the lncRNA is an ASO comprising a nucleic acid sequence having at least about 70% sequence identity to SEQ ID NO: 10 or SEQ ID NO: 11. In some embodiments, the subject in need thereof has or is suspected to have malignant glioma. In some embodiments, the malignant glioma is glioblastoma or diffuse intrinsic pontine gliomas. In some embodiments, the subject in need thereof is concurrently under radiation therapy. In some embodiments, administration of the agent that knockdowns expression of the lncRNA increases or potentiates the effect of the radiation therapy as compared to administering the radiation therapy alone.

In one aspect, the present disclosure relates to a method of inhibiting growth and/or proliferation of glioma cells in a subject in need thereof comprising administering to the subject a therapeutically effective amount of an agent that knockdowns expression of a long noncoding RNA (lncRNA) having at least about 70% sequence identity to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 and SEQ ID NO: 9. In some embodiments, the agent that knockdowns expression of the lncRNA is a miRNA, siRNA, antisense oligonucleotide (ASO), or morpholino. In some embodiments, the lncRNA comprises a nucleic acid sequence having at least about 70% sequence identity to SEQ ID NO: 1. In some embodiments, the lncRNA comprises the nucleic acid sequence of SEQ ID NO: 1. In some embodiments, the agent that knockdowns expression of the lncRNA is an ASO comprising a nucleic acid sequence having at least about 70% sequence identity to SEQ ID NO: 10 or SEQ ID NO: 11. In some embodiments, the subject in need thereof has or is suspected to have malignant glioma. In some embodiments, the malignant glioma is glioblastoma or diffuse intrinsic pontine gliomas. In some embodiments, the subject in need thereof is concurrently under radiation therapy. In some embodiments, administration of the agent that knockdowns expression of the lncRNA increases the effect of the radiation therapy compared to administering the radiation therapy alone.

In another aspect, the present disclosure relates to a pharmaceutical composition comprising a therapeutically effective amount of an agent that knockdowns expression of a long noncoding RNA (lncRNA) having at least about 70% sequence identity to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 AND SEQ ID NO: 9 and a pharmaceutically acceptable carrier. In some embodiments, the pharmaceutic composition of the present disclosure is for treating malignant glioma in a subject in need thereof In some embodiments, the agent that knockdowns expression of the lncRNA is a miRNA, siRNA, antisense oligonucleotide (ASO), or morpholino. In some embodiments, the lncRNA comprises a nucleic acid sequence having at least about 70% sequence identity to SEQ ID NO: 1. In some embodiments, the lncRNA comprises the nucleic acid sequence of SEQ ID NO: 1. In some embodiments, the agent that knockdowns expression of the lncRNA is an ASO comprising a nucleic acid sequence having at least about 70% sequence identity to SEQ ID NO: 10 or SEQ ID NO: 11. In some embodiments, the malignant glioma is glioblastoma or diffuse intrinsic pontine gliomas.

In a further aspect, the present disclosure relates to a method for generation of a human brain organoid model of malignant glioma comprising: a) seeding cells derived from glioblastoma (GBM) or diffuse intrinsic pontine gliomas (DIPG) onto a surface of a three-dimensional human brain organoid having one or more aspects of a mature human brain; b) allowing the cells to grow and form a tumor within the human brain organoid. In some embodiments, the three-dimensional human brain organoid comprises mature human astrocytes from human iPSCs (iAstrocytes) and/or mature cortical neurons with NGN2 induction (i3Neurons). In some embodiments, the three-dimensional human brain organoid comprises solely iAstrocytes or solely i3Neurons. In some embodiments, the three-dimensional human brain organoid comprises iAstrocytes and i3Neurons in a ratio of about 1:1. In some embodiments, the cells derived from GBM are GBM SF10360 cells. In some embodiments, the cells derived from DIPG are DIPG SF8628 cells.

In another aspect, the present disclosure further relates to a human brain organoid model of malignant glioma obtained by the method disclosed herein.

In yet another aspect, the present disclosure also relates to a method of screening an agent capable of synergizing with radiation to reduce growth of glioma cells comprising: a) adding a candidate agent to the human brain organoid model of malignant glioma according to the present disclousre; b) treating the human brain organoid model of malignant glioma with a clinically therapeutic level of radiation; and c) detecting the effectiveness of the candidate agent in synergizing with the radiation to reduce growth of glioma cells by comparing with a corresponding control treated with the radiation alone without addition of the candidate agent.

In some embodiments, the human brain organoid model of malignant glioma is treated with a fractionated radiation of up to about 54 Gy. In some embodiments, the human brain organoid model of malignant glioma is treated with about 8 Gy fractionated radiation in about 4, 2 or 1 fraction. In some embodiments, the candidate agent is an agent that knockdowns expression of a lncRNA. In some embodiments, the agent that knockdowns expression of the lncRNA is a miRNA, siRNA, antisense oligonucleotide (ASO), or morpholino. In some embodiments, the lncRNA comprises a nucleic acid sequence having at least about 70% sequence identity to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 and SEQ ID NO: 9. In some embodiments, the lncRNA comprises the nucleic acid sequence of SEQ ID NO: 1. In some embodiments, the agent is an ASO comprising a nucleic acid sequence having at least about 70% sequence identity to SEQ ID NO: 10 or SEQ ID NO: 11.

In another aspect, the present disclosure relates to a method of treating malignant glioma in a subject in need thereof, said method comprising: a) exposing a sample from the subject to a nucleic acid complementary to a long noncoding RNA (lncRNA) comprising a nucleic acid sequence having at least about 70% sequence identity to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 and SEQ ID NO: 9; b) detecting binding between a lncRNA the sample and the nucleic acid complementary to the lncRNA, wherein a positive interaction between the sample and the nucleic acid complementary to the lncRNA is indicative of said subject having malignant glioma; and c) providing the subject a treatment regime to manage the malignant glioma.

In some embodiments, the sample used in the disclosed method is a human tissue sample, serum, plasma, blood draw, brushing, biopsy, or surgical resection of the subject. In some embodiments, the interaction between the sample and the nucleic acid complementary to the lncRNA is detected by fluorescence, radioactivity, enzyme-linked electrochemical, or chemoluminescence. In some embodiments, the lncRNA comprises a nucleic acid sequence having at least about 70% sequence identity to SEQ ID NO: 1. In some embodiments, the lncRNA comprises the nucleic acid sequence of SEQ ID NO: 1. In some embodiments, the treatment regime to manage the malignant glioma comprises a radiotherapy. In some embodiments, the malignant glioma is glioblastoma or diffuse intrinsic pontine gliomas. In some embodiments, the treatment regime to manage the malignant glioma comprises administering a therapeutically effective amount of an agent that knockdowns expression of a long noncoding RNA (lncRNA) having at least about 70% sequence identity to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 and SEQ ID NO: 9. In some embodiments, the agent that knockdowns expression of the lncRNA is a microRNA (miRNA), small interfering RNA (siRNA), antisense oligonucleotide (ASO), or morpholino. In some embodiments, the agent that knockdowns expression of the lncRNA is an ASO comprising a nucleic acid sequence having at least about 70% sequence identity to SEQ ID NO: 10 or SEQ ID NO: 11.

In a further aspect, the present disclosure relates to a method of treating brain cancer in a subject in need thereof comprising administering to the subject a therapeutically effective amount of an agent that knockdowns expression of a long noncoding RNA (lncRNA) having at least about 70% sequence identity to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 and SEQ ID NO: 9. In some embodiments, the agent that knockdowns expression of the lncRNA is a microRNA (miRNA), small interfering RNA (siRNA), antisense oligonucleotide (ASO), or morpholino. In some embodiments, the lncRNA comprises a nucleic acid sequence having at least about 70% sequence identity to SEQ ID NO: 1. In some embodiments, the lncRNA comprises the nucleic acid sequence of SEQ ID NO: 1. In some embodiments, the agent that knockdowns expression of the lncRNA is an ASO comprising a nucleic acid sequence having at least about 70% sequence identity to SEQ ID NO: 10 or SEQ ID NO: 11. In some embodiments, the subject is concurrently under radiation therapy. In some embodiments, administration of the agent that knockdowns expression of the lncRNA increases the effect of the radiation therapy compared to administering the radiation therapy alone.

In another aspect, the present disclosure relates to a method of identifying a radiotherapy sensitizer comprising: a) exposing a plurality of test cells to a radiation, the plurality of test cells each comprising: i) a small guide RNA (sgRNA) that targets a locus encoding a long non-coding RNA; and ii) a nuclease-deficient sgRNA-mediated nuclease (dCas9), wherein the dCas9 comprises a dCas9 domain fused to a transcriptional modulator, b) obtaining a radiation modifier screen score of each test cell, wherein the radiation modifier screen score is an average phenotype of top three sgRNAs against a given gene multiplied by the negative log₁₀(Mann-Whitney-U p value) for said given gene in the test cell treated with radiation; c) obtaining a growth screen score of each test cell that is not irradiated, wherein the growth screen score is an average phenotype of top three sgRNAs against said given gene multiplied by the negative log₁₀(Mann-Whitney-U p value) for said given gene in the test cell that is not irradiated; d) selecting a test cell having a sensitizer score that is greater than about 1, wherein the sensitizer score is a ratio of the radiation modifier screen score to the growth screen score; and e) identifying the locus targeted by the sgRNA in the test cell selected in d) as the radiotherapy sensitizer.

In some embodiments, the test cells are glioblastomas (GBM) cells. In some embodiments, the transcriptional modulator is a transcriptional activator or a transcriptional repressor. In some embodiments, the dCas9 comprises an amino acid sequence having at least about 70% sequence identity to the amino acid sequence of SEQ ID NO: 12, SEQ ID NO: 13, SEQ ID NO: 14 or SEQ ID NO: 15. In some embodiments, the radiation used to expose the cells is at a total dose of from about 1 Gy to about 12 Gy and delivered in about 1 to about 6 fractions. In some embodiments, the radiation used to expose the cells is at a total dose of about 8 Gy and delivered in about 4 fractions. In some embodiments, the radiotherapy sensitizer comprises a nucleic acid sequence having at least about 70% sequence identity to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 and SEQ ID NO: 9.

In some embodiments, any of the methods disclosed herein further comprises a step of correlating the binding between the lncRNA in the sample and the nucleic acid complementary to the lncRNA to the subject having malignant glioma. In some embodiments, the nucleic acid complementary to the lncRNA useful for any of the disclosed methods comprises at least about 70% sequence identity to any of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 and SEQ ID NO: 46, or a functional fragment thereof.

In some embodiments, the gent that knockdowns expression of the lncRNA disclosed herein comprises at least about 70% sequence identity to any of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 and SEQ ID NO: 46, or a functional fragment thereof. In some embodiments, the gent that knockdowns expression of the lncRNA disclosed herein is a fragment of any of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 and SEQ ID NO: 46, wherein the fragment comprises from about 10 to about 100 nucleotides. In some embodiments, the gent that knockdowns expression of the lncRNA disclosed herein is a miRNA, a siRNA or an ASO comprising at least about 70% sequence identity to any of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 and SEQ ID NO: 46, or a functional fragment thereof In some embodiments, the gent that knockdowns expression of the lncRNA disclosed herein is a miRNA, a siRNA or an ASO ASO comprising a fragment of any of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 and SEQ ID NO: 46, wherein the fragment comprises from about 10 to about 100 nucleotides. In some embodiments, the gent that knockdowns expression of the lncRNA disclosed herein comprises one or a plurality of modified nucleotides.

The disclosure further relates to a method of preparing a sample from a subject suspected of having malignant glioma comprising: a) obtaining the sample from the subject; b) isolating total RNA from the sample; and c) analyzing the total RNA with a probe specific for one or a plurality of long noncoding RNAs (lncRNAs) comprising a nucleic acid sequence having at least about 70% sequence identity to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 and SEQ ID NO: 9. In some embodiments, the sample is a human tissue sample, serum, plasma, blood draw, brushing, biopsy, or surgical resection of the subject. In some embodiments, the probe specific for the one or plurality of lncRNAs comprises at least about 70% sequence identity to any of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 and SEQ ID NO: 46, or a functional fragment thereof. In some embodiments, the probe specific for the one or plurality of lncRNAs is a fragment of any of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 and SEQ ID NO: 46, wherein the fragment comprises from about 10 to about 100 nucleotides. In some embodiments, the method further comprises: a) detecting the presence, absence or quantity of the one or plurality of lncRNAs in the sample; b) normalizing the presence, absence or quantity of the one or plurality of lncRNAs in the sample against the presence, absence or quantity of the one or plurality of lncRNAs in a sample of a healthy subject; and c) correlating the presence, absence or quantity of the one or plurality of lncRNAs in the sample to the subject having malignant glioma.

The disclosure further relates to a system comprising: a) one or a plurality of nucleic acids complementary to, or substantially complementary to, one or a plurality of lncRNAs chosen from nucleic acid sequences comprising at least about 70% sequence identity to one or a plurality of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 and SEQ ID NO: 9; and b) a solid support onto to which the one or plurality of nucleic acids are immobilized. In some embodiments, the one or plurality of nucleic acids comprised in the disclosed system comprises at least about 70% sequence identity to one or a plurality of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 and SEQ ID NO: 46, or a functional fragment thereof. In some embodiments, the one or plurality of nucleic acids comprised in the disclosed system are fragments of any of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 and SEQ ID NO: 46, wherein the fragment comprises from about 10 to about 100 nucleotides.

### BRIEF DESCRIPTION OF THE DRAWINGS

FIG. 1A-1C depict a glioma cell culture model for identifying radiation sensitizers. FIG. 1A: Doubling time of U87 GBM cells in culture after treatment with different amounts of single dose or fractionated radiation. Fractionated (fx) radiation (8 Gy in 4 fx) was delivered in 2 Gy doses every other day (n=3 biological replicates per condition; error bar=S.D.). FIG. 1B-1C: Internally controlled growth assays for U87 cells evaluating CRISPRi knockdown of ERCC6L2 with (FIG. 1B) and without (FIG. 1C) radiation (n=3 biological replicates per condition; error bar=S.D.; two tailed student's t test of end points; radiation delivery time points indicated below the x axis).

FIG. 2A-2B depict transcriptomic analysis of radiation response. FIG. 2A: RNA-seq analysis of differentially expressed genes following single dose radiation of U87 cells, along with significant gene ontology terms for upregulated and downregulated genes (n=2-3 biological replicates per condition). FIG. 2B: Log 2 fold change of lncGRS-1 in U87 cells following radiation, averaged across all replicates from the same condition.

FIG. 3A-3D depict CRISPRi radiation modifier screen in glioma cells. FIG. 3A: Experimental design. 8 Gy radiation was delivered in 4 fractions throughout the screen. FIG. 3B: Volcano plot of radiation growth phenotypes (average of two replicate screens) for the top 3 sgRNAs targeting each lncRNA TSS (x axis) and negative log₁₀(Mann-Whitney-U p value) of all sgRNAs for a given gene at T12 as compared to T0 (y axis). Screen threshold was determined by the product of the x and y axes that resulted in an empirical FDR of 0.0025. Neighbor hits are defined as any lncRNA hit with an expressed protein coding gene within 1 kb of the lncRNA TSS. Phenotype refers to the relative log 2 enrichment of barcodes in the final timepoint divided by the enrichment of barcodes at the initial timepoint [8,11]. FIG. 3C: Comparison of screen scores, defined as the average phenotype of the top three sgRNAs against a given gene multiplied by the negative log₁₀(Mann-Whitney-U p value) for that gene, from screens conducted without radiation (x-axis) with scores from screens conducted with radiation (y-axis), with thresholds set at 5 (FDR=0.0025). Thirty-three lncRNA hits had radiation screen scores greater than no radiation screen scores. CTC-338M12.4 (lncGRS-1) is indicated. FIG. 3D: LncRNA expression across 2 DIPG and 3 GBM cultures (subpanel 1), no radiation screen scores (subpanel 2), radiation screen scores (subpanel 3), and sensitizer scores (subpanel 4) for each of the 9 lncGRS candidates. Sensitizer score is defined as the ratio of the radiation modifier screen score in irradiated cells to the growth screen score in non-irradiated cells.

FIG. 4A-4C depict the properties of the CRISPRi radiation modifier screen. FIG. 4A: Proportion of two replicates of the screen population that are expressing sgRNA (BFP positive). Puromycin selection time period highlighted in yellow. Radiation doses indicated by arrows. FIG. 4B: Z standardized growth (no radiation) and radiation phenotypes for PVT1 in CRISPRi screens. FIG. 4C: Comparison of z standardized radiation phenotypes (x-axis) and log 2 fold change of targeted lncRNA expression from RNA-seq (y-axis) analysis. Z standardized phenotypes were calculated as log 2 enrichment normalized by the standard deviation of negative control genes' phenotypes. lncGRS-1 to lncGRS-9 are labeled by their NCBI gene names.

FIG. 5A-5J show that lncGRS-1 is a primate-conserved radiation sensitizer target in glioma. FIG. 5A: Nanopore direct RNA-seq spliced reads aligned to the lncGRS-1 gene body in U87 cells, with GENCODE v29 transcript models of lncGRS-1 (CTC-338M12.4) and multiz alignment for conservation. FIG. 5B: Subcellular fractionation followed by qPCR of transcripts in U87 cells. FIG. 5C: Single molecule FISH of lncGRS-1 in GBM SF10360 and DIPG SF8628 primary glioma cells (scale bar=5 μm). FIG. 5D: RT-qPCR for lncGRS-1 after CRISPRi targeting in U87 (n=2 biological replicates; error bar=S.D.). FIG. 5E-5F: Internally controlled growth assays of U87 cells with CRISPRi knockdown of lncGRS-1 in the absence (FIG. 5E) and presence (FIG. 5F) of fractionated radiation. FIG. 5G-5H: Cell propagation assay of purified populations of U87 cells with lncGRS-1 CRISPRi knockdown in the absence (FIG. 5G) and presence (FIG. 5H) of fractionated radiation. FIG. 5I-5J: Cell propagation assay of purified populations of HeLa cells with lncGRS-1 CRISPRi knockdown in the absence (FIG. 5I) and presence (FIG. 5J) of fractionated radiation. For FIG. 5E-5J, n=2 biological replicates per condition; error bars=S.D.; two tailed student's t test. N.S.=not significant. Radiation delivery time points indicated below the x axis.

FIG. 6 shows that nanopore direct RNA-seq of spliced reads aligned to the lncGRS-1 through lncGRS-9 loci in U87 cells, with GENCODE v29 transcript models, Ensembl H3K27Ac layered track, and multiz alignment for conservation (from top to bottom in each subpanel).

FIG. 7A-7D show that lncGRS-1 is required for the proliferation of primary, patient-derived glioma cells. FIG. 7A: RT-qPCR of lncGRS-1 transcript levels following CRISPRi-mediated knockdown in GBM SF10360 and DIPG SF8628 (n=2 biological replicates per condition; error bars=S.D.). FIG. 7B: Internally controlled growth assays of GBM SF10360 and DIPG SF8628 cells with CRISPRi-mediated knockdown of lncGRS-1 (n=2 biological replicates per condition; error bars=S.D.). FIG. 7C: RT-qPCR of lncGRS-1 transcript levels following ASO-mediated knockdown of in GBM SF10360 and DIPG SF8628 (n=2 biological replicates per condition; error bars=S.D.). FIG. 7D: Cell propagation time course of GBM SF10360 and DIPG SF8628 cells with ASO-mediated knockdown of lncGRS-1. ASOs were re-transfected at day 7 (n=2 biological replicates per condition; error bars=S.D.).

FIG. 8A-8I depict ASO knockdown of lncGRS-1 demonstrating glioma specific phenotype. FIG. 8A: Single molecule RNA FISH of lncGRS-1 in DIPG SF8628 cells following transfection of non-targeting ASO (top) or ASO targeting lncGRS-1 (bottom). Scale bar=5 FIG. 8B: RT-qPCR of TP53 (p53) transcript levels following ASO knockdown of TP53 in U87 cells. FIG. 8C: lncGRS-1 locus with locations of sgRNA, ASO, and qPCR primer targets. FIG. 8D-8G: RT-qPCR of lncGRS-1 transcript levels (left) and cell propagation assay (right) following ASO knockdown of lncGRS-1 in SU-DIPG 24 (FIG. 8D), SU-DIPG 25 (FIG. 8E), GBM 43 (FIG. 8F), and HEK293T cells (FIG. 8G). FIG. 8H: RT-qPCR of POLA1 transcript levels (left) and cell proliferation assay (right) following ASO knockdown of NHA cells (at day 7) or in (FIG. 8I) U87 cells (at day 3). n=2-3 biological replicates per condition in all experiments indicated; error bar=S.D.

FIG. 9A-9E show that lncGRS-1 function is glioma specific. FIG. 9A: RT-qPCR of lncGRS-1 transcript levels following ASO-mediated knockdown in NHA cells (n=2 biological replicates per condition; error bars=S.D.). FIG. 9B: Cell propagation time course in NHA cells with ASO-mediated knockdown of lncGRS-1. ASOs were re-transfected at day 7 (n=2 biological replicates per condition; error bars=S.D.). FIG. 9C: Left, fluorescence viability assay of malignant tumor cells and NHA cells following ASO-mediated lncGRS-1 knockdown. Right, apoptosis assay of malignant tumor cells and NHA cells following ASO-mediated lncGRS-1 knockdown (n=2 biological replicates per condition; error bars=S.D.). FIG. 9D: Cell cycle phase analysis following lncGRS-1 knockdown in GBM U87 using flow cytometry. FIG. 9E: RNA-seq differential gene expression analysis of lncGRS-1 knockdown in GBM U87 (left), DIPG SF8628 (middle), and NHA (right) cells using lncGRS-1 ASO #1 and ASO #2 as biological replicates, compared to negative control ASO, 24 hours following transfection (n=2 biological replicate cultures per ASO condition). Green, genes adj. p val.<0.05. Red triangle, lncGRS-1.

FIG. 10A: Cell propagation assay of purified populations of HeLa cells with lncGRS-1 CRISPRi knockdown. FIG. 10B: Expression values (log 2 (TPM+1)) of IncGRS-1 across cell lines in the CCLE atlas, grouped by disease of origin or tissue type. FIG. 10C: Top 5 gene ontology terms for upregulated (top) and downregulated (bottom) differentially expressed genes with adj. p val<0.05, in GBM U87 (left) and DIPG SF8628 (right) 24 hours following lncGRS-1 ASO-mediated knockdown. FIG. 10D: Scatter plot of genes differentially expressed in either U87 or SF8628 cell lines demonstrating positive correlation in expression changes following lncGRS-1 knockdown. FIG. 10E: RNA-seq expression values and (FIG. 10F) western blot of protein levels for CDKN1A (p21) with quantification (right) in U87 cells following lncGRS-1 knockdown. FIG. 10G: Immunohistochemistry of p53BP1 and (FIG. 10H) γH2AX nuclear foci in nuclei of U87 cells following lncGRS-1 knockdown with or without 2 Gy radiation. Scale bar=5 μm. n=range of 225 to 440 nuclei per replicate across 2 biological replicates per condition.

FIG. 11A-11J depict tumor specific, radiosensitizing function of lncGRS-1 knockdown in mature brain organoids (MBOs). FIG. 11A: Schematic of MBO assembly from induced mature neurons (iAstrocytes) and induced neurons (i³Neurons). FIG. 11B: Single molecule RNA FISH of lncGRS-1 in iAstrocyte MBO (A-MBO) cells following transfection of non-targeting ASO (top) or ASO targeting lncGRS-1 (bottom) (scale bar=5 μm). FIG. 11C: RT-qPCR of lncGRS-1 in A-MBOs seeded with DIPG SF8628 following ASO transfection (n=2 biological replicates per condition; error bar=S.D.). FIG. 11D: Left, fluorescence viability assay of A-MBOs following transfection of non-targeting ASO or ASO targeting lncGRS-1. Right, apoptosis induction assay of A-MBOs following transfection of ASOs (n=2 biological replicates per condition; error bar=S.D.). FIG. 11E: Schematic of RFP+ glioma cell seeding and subsequent RFP+ tumor growth over time. FIG. 11F-11G: Longitudinal fluorescence microscopy of A-MBOs seeded with RFP+ DIPG SF8628 cells treated with non-targeting Ctrl ASO (FIG. 11F) or ASO targeting lncGRS-1 (FIG. 11G). FIG. 11H-11I: Quantification of RFP+ tumor burden (FIG. 11H) and organoid diameter (FIG. 11I) in longitudinal analysis of A-MBOs seeded with RFP+ DIPG SF8628 cells (n=6 biological replicates per condition; two tailed student's t test). FIG. 11J: Quantification of RFP+ U87 GBM tumor burden in iAstrocyte and iNeuron MBOs (AN-MBOs) treated with ASOs with or without radiation (n=5 biological replicates per condition; two tailed student's t test). Boxplots represents 1^{st} quartile, median, and 3^{rd} quartile with whiskers=range.

FIG. 12A-12E depict radiosensitization of glioma cells in MBO hosts. FIG. 12A: Quantification of single molecule RNA FISH of lncGRS-1 in iAstrocyte MBO (A-MBO) nuclei following transfection of non-targeting ASO or ASO targeting lncGRS-1. n=69 and 98 A-MBO nuclei quantified in ASO-Ctrl and ASO #2 conditions, respectively, across 2 independent experiments for each biological condition. FIG. 12B: Left, fluorescence viability assay of combined (1:1 ratio) iAstrocyte and i³Neuron organoids (AN-MBO) following transfection of non-targeting ASO or ASO targeting lncGRS-1. Right, apoptosis induction assay of AN-MBOs following transfection of non-targeting ASO or ASO targeting lncGRS-1 (n=3 biological replicates per condition; error bar=S.D.). FIG. 12C: Fold change in AN-MBO size between day 2 and day 21 of co-culture with growth arrested DIPG SF8628 cells, with negative control or lncGRS-1 ASO, at various doses of fractionated radiation (n=5 biological replicates per condition; boxplot represents 1^{st} quartile, median, and 3^{rd} quartile with whiskers=range). FIG. 12D: Confocal microscopy of AN-MBO 20 days following seeding of RFP+ U87 glioma cells. Nuclei are counterstained with DAPI (scale bar=100 μm). FIG. 12E: Longitudinal fluorescence microscopy of individual AN-MBOs seeded with RFP+ U87 cells. Cultures were treated with non-targeting ASO (Ctrl) or ASO targeting lncGRS-1 combined with 0 Gy, 12 Gy, or 18 Gy of fractionated radiation.

FIG. 13 shows full size western blot with additional replicate, corresponding to FIG. 10F.

### DETAILED DESCRIPTION OF EMBODIMENTS

The present disclosure can be understood more readily by reference to the following detailed description of embodiments, the figures and the examples included herein.

Before the present methods and compositions are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

Unless defined otherwise, all technical and scientific terms used herein have the same meaning as commonly understood by one of ordinary skill in the art to which the invention pertains.

As used in the specification and in the claims, the term “comprising” can include the aspects “consisting of” and “consisting essentially of” Comprising can also mean “including but not limited to.”

As used in the specification and the appended claims, the singular forms “a,” “an” and “the” can include plural referents unless the context clearly dictates otherwise. Thus, for example, reference to “a compound” includes mixtures of compounds; reference to “a pharmaceutical carrier” includes mixtures of two or more such carriers, and the like.

The word “or” as used herein means any one member of a particular list and also includes any combination of members of that list.

The term “about” is used herein to mean within the typical ranges of tolerances in the art. For example, “about” can be understood as about 2 standard deviations from the mean. According to certain embodiments, when referring to a measurable value such as an amount and the like, “about” is meant to encompass variations of ±20%, ±10%, ±5%, ±1%, ±0.9%, ±0.8%, ±0.7%, ±0.6%, ±0.5%, ±0.4%, ±0.3%, ±0.2% or ±0.1% from the specified value as such variations are appropriate to perform the disclosed methods. When “about” is present before a series of numbers or a range, it is understood that “about” can modify each of the numbers in the series or range.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) J. Biol. Chem. 260:2605-2608; and Rossolini et al. (1994) Mol. Cell. Probes 8:91-98). The term nucleic acid is used interchangeably with gene, cDNA, and mRNA encoded by a gene.

The term “biomarker” as used herein refers to a biological molecule present in an individual at varying concentrations useful in predicting the cancer status of an individual. A biomarker may include but is not limited to, nucleic acids, proteins and variants and fragments thereof. A biomarker may be an RNA or cDNA complement of the RNA molecule comprising the entire or partial nucleic acid sequence encoding the biomarker, or the complement of such a sequence. Biomarker nucleic acids useful in the present disclosure are considered to include both DNA and RNA comprising the entire or partial sequence of any of the nucleic acid sequences of interest. In some embodiments, the disclosure relates to a composition comprising cDNA sequences that are complementary to one or a plurality of RNA sequences chosen from: SEQ ID NO:1, SEQ ID NO:2, SEQ ID NO:3, SEQ ID NO:4, SEQ ID NO:5, SEQ ID NO:6 SEQ ID NO:7, SEQ ID NO:8, and SEQ ID NO:9. In some embodiments, the disclosure relates to a composition or a kit comprising one or a plurality of containers that comprise RNA complementary to the above-identified sequence identifiers or DNA (particularly cDNA) complementary to the above-identified sequence identifiers, wherein the RNA complementary to the above-identified sequence identifiers or DNA (particularly cDNA) complementary to the above-identified sequence identifiers are in each independently in one container a plurality of those RNAs or cDNAs mixed in another container. In some embodiments, RNA complementary to the above-identified sequence identifiers or DNA (particularly cDNA) complementary to the above-identified sequence identifiers

The term “small non-coding RNA” (ncRNA) as used herein refers to RNA that is not translated into protein and includes transfer RNA (tRNA), ribosomal RNA (rRNA), snoRNAs, microRNA (miRNA), siRNAs, small nuclear (snRNA), Y RNA, vault RNA, antisense RNA, tiRNA (transcription initiation RNA), TSSa-RNA (transcriptional start-site associated RNA) and piwiRNA (piRNA). Small ncRNA have a length of less than 200 nucleotides. Preferably, a small ncRNA as used herein is between 50 and 100 nucleotides. A ncRNA may be of endogenous origin (e.g., a human small non-coding RNA) or exogenous origin (e.g., virus, bacteria, parasite). “Canonical” ncRNA refers to the sequence of the RNA as predicted from the genome sequence and is the most abundant sequence identified for a particular RNA. “Trimmed” ncRNA refers to an ncRNA in which exonuclease-mediated nucleotide trimming has removed one or more nucleotides at the 5′ and/or 3′ end of the molecule. “Extended ncRNA” refers to an small non-coding RNA that is longer than the canonical small non-coding RNA sequence and is a term recognized in the art. The nucleotides making up the extension correspond to nucleotides of the precursor sequence and are therefore encoded by the genome in contrast to non-templated nucleotide addition.

The term “long noncoding RNA” or “lncRNA” refers to a functional RNA molecule or transcript with length exceeding about 200 nucleotides that is not translated into protein (Perkel J. M., BioTechniques, 2013, 54(6): 301, 303-4). LncRNAs are located within the intergenic stretches or overlapping antisense transcripts of protein coding genes.

The term “locus” is defined as a segment of DNA within the genomic DNA. For example, a lncRNA locus is a segment of DNA within the genomic DNA that encodes a lncRNA. Thus, as used herein, the term “a locus encoding a long non-coding RNA” refers to the DNA sequence from which a non-coding RNA is transcribed, which is often called in the art an RNA gene.

The term “microRNA” or “miRNA” as used herein refers to a small non-coding RNA molecule containing about 22 nucleotides that functions in RNA silencing and post-transcriptional regulation of gene expression. MicroRNAs function via base-pairing with complementary sequences within mRNA molecules. As a result, these mRNA molecules are silenced, by cleavage of the mRNA strand into two pieces, destabilization of the mRNA through shortening of its poly-A tail, and/or less efficient translation of the mRNA into proteins by ribosomes.

As used herein, the term “small interfering RNA” or “siRNA,” sometimes known as short interfering RNA or silencing RNA, refers to a class of double-stranded, noncoding RNA molecules of about 20-25 base pairs in length, similar to miRNA, which operate within the RNA interference (RNAi) pathway. They interfere with the expression of specific genes with complementary nucleotide sequences by degrading mRNA after transcription, thus preventing translation.

“Antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound. In certain embodiments, antisense activity is a change in splicing of a pre-mRNA nucleic acid target. In certainembodiments, anti sense activity is an increase in the amount or expression of a target nucleic acid or protein encoded by such target nucleic acid compared to target nucleic acid levels or target protein levels in the absence of the antisense compound.

“Antisense compound” means a compound comprising an antisense oligonucleotide and optionally one or more additional features, such as a conjugate group or terminal group.

The term “antisense oligonucleotide” or “ASO” as used herein refers to an oligomeric nucleic acid that is capable of hybridizing with its complementary target sequence, generally resulting in the modulation of the normal function of the nucleic acid (e.g., mRNA) having the target sequence. Thus, “antisense oligonucleotide” means an oligonucleotide that (1) has a nucleotide sequence that is at least partially complementary to a target nucleic acid and that (2) is capable of producing an antisense activity in a cell or animal. “Antisense” further refers to an oligomer having a sequence of nucleotide bases and a subunit-to-subunit backbone that allows the antisense oligomer to hybridize to a target sequence in an RNA by Watson-Crick base pairing, to form an RNA: oligomer heteroduplex within the target sequence, typically with an mRNA. The antisense oligomer (oligonucleotide) may have exact sequence complementarity to the target sequence or near complementarity, and may include modified (non-natural) nucleobases in place of naturally complementary nucleobases.

The term “morpholino,” also known as “morpholino oligomer” or “phosphorodiamidate morpholino oligomer” (PMO), as used herein, refers to a nucleic acid base structure comprising a chain of A, T, G, and/or C bases having a backbone of methylenemorpholine rings linked through phosphorodiamidate groups. The morpholinos of the present disclosure also include variants thereof which comprise modified nucleobases which do not substantially diminish their affinity for the target epitope of the target nucleic acid molecules, such as (but not limited to) mRNA. For example, variants include, but are not limited to, morpholinos which are the same as the morpholinos described herein except having at least one base substitution (e.g., A for T, T for A, C for G, and G for C) which does not substantially impair the agonistic or antagonistic activity or properties of the variants described herein. Further, variant bases may comprise modified or non-natural purine and pyrimidine bases such as described herein above. The morpholinos of the present disclosure may have about 15 to 50, or 18 to 40, or 20 to 30, or 22 to 28 nucleotides.

As used herein, the term “complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. “Complementary” in reference to an oligonucleotide means that at least 70% of the nucleobases of such oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases means nucleobases that are capable of forming hydrogen bonds with one another. Complementary nucleobase pairs include, but unless otherwise specific are not limited to, adenine (A) and thymine (T), adenine (A) and uracil (U), cytosine (C) and guanine (G), 5-methyl cytosine (mC) and guanine (G). Complementary oligonucleotides and/or nucleic acids need not have nucleobase complementarity at each nucleoside. Rather, some mismatches are tolerated. Two single-stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, at least about 75%, or at least about 90% complementary. See Kanehisa (1984) Nucl. Acids Res. 12:203.

“Hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or eversed Hoogsteen hydrogen bonding, between complementary nucleobases.

As used in herein, the terms “identical” or percent “identity”, in the context of describing two or more polynucleotide or amino acid sequences, refer to two or more sequences or subsequences that are the same or have a specified percentage of nucleotides or amino acid residues that are the same. With regard to polynucleotide sequences, this definition also refers to the complement of a test sequence. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.0 algorithms and the default parameters discussed below are used.

As used herein, the term “radiotherapy sensitizer” refers to a locus, such as an lncRNA locus in a smaple from a subject, the knockdown of which enhances the effects of radiation therapy. Thus, a combination of radiotherapy sensitizer knockdown and radiation can significantly decrease tumor cell propagation or proliferation as compared to radiotherapy alone. In some embodiments, the methods of the disclosure relate to a method of identifying a radiotherapy sensitizer who has been diagnosed with malignant glioma and then treating that subject with: (i) radiotherapy or (ii) the combination of radiotherapy and an agent that interferes with the function of one or a combination of the lncRNAs of the disclosure. In some embodiments, the agent that interferes with theh function of one or a combination of the lncRNAs of the disclosure is an siRNA, cDNA or miRNA that is complementary to the one or plurality of lncRNAs of the disclosure.

The “CRISPR/Cas” system refers to a widespread class of bacterial systems for defense against foreign nucleic acid. CRISPR/Cas systems are found in a wide range of eubacterial and archaeal organisms. CRISPR/Cas systems include type I, II, and III sub-types. Wild-type type II CRISPR/Cas systems utilize the RNA-mediated nuclease, Cas9 in complex with guide and activating RNA to recognize and cleave foreign nucleic acid. Methods and compositions for controlling inhibition and/or activation of transcription of target genes, populations of target genes (e.g., controlling a transcriptome or portion thereof) are described, e.g., in Cell, 2014 Oct. 23, 159(3):647-61, the contents of which are incorporated by reference in the entirety for all purposes.

Cas proteins are endonuclease that form part of an adaptive defense mechanism evolved by bacteria and archaea to protect them from invading viruses and plasmids. Cas9 protein is the major protein element of the CRISPR/Cas9 system, which forms a complex with crRNA (CRISPR RNA) and tracrRNA (trans-activating crRNA) to form activated endonuclease or nickase. “CRISPR system” refers collectively to transcripts or synthetically produced transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous

CRISPR system). In the context of formation of a CRISPR complex, “target sequence” refers to a nucleic acid sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In some embodiments, the target sequence is a DNA polynucleotide and is referred to a DNA target sequence. In some embodiments, a target sequence comprises at least three nucleic acid sequences that are recognized by a Cas-protein when the Cas protein is associated with a CRISPR complex or system which comprises at least one sgRNA or one tracrRNA/crRNA duplex at a concentration and within an microenvironment suitable for association of such a system. In some embodiments, the target DNA comprises at least one or more proto-spacer adjacent motifs which sequences are known in the art and are dependent upon the Cas protein system being used in conjunction with the sgRNA or crRNA/tracrRNAs employed by this work. In some embodiments, the target DNA comprises NNG, where G is a guanine and N is any naturally occurring nucleic acid. In some embodiments the target DNA comprises any one or combination of NNG, NNA, GAA, NNAGAAW and NGGNG, where G is an guanine, A is adenine, and N is any naturally occurring nucleic acid.

The term “Cas9 protein” refers to the “clustered, regularly interspaced, short palindromic repeats (CRISPR)-associated protein 9.” This term is well known in the art and has been described, e.g. in Makarova et al. (2011) Nat. Rev. Microbiol., 9:467-477, and in Makarova et al. (2011) Biol. Direct., 6:38. Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chlroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi, et al., RNA Biol. 2013 May 1; 10(5): 726-737; Nat. Rev. Microbiol. 2011 June; 9(6): 467-477; Hou, et al., Proc Natl Acad Sci USA. 2013 Sep. 24; 110(39):15644-9; Sampson et al., Nature. 2013 May 9; 497(7448):254-7; and Jinek, et al., Science. 2012 Aug. 17; 337(6096):816-21. Cas9 protein or gene information can be obtained from a known database such as the GenBank of NCBI (National Center for Biotechnology Information), but is not limited thereto. Moreover, the Cas9 protein may comprise not only wild-type Cas9, but also Cas9 variants such as Cas9 nickase. The Cas9 protein is not limited in its origin. For example, the Cas9 protein may be derived from Streptococcus pyogenes, Francisella novicida, Streptococcus thermophilus, Legionella pneumophila, Listeria innocua, or Streptococcus mutans.

The term “nuclease-deficient sgRNA-mediated nuclease” or “dCas9” refers to a catalytically deactivated, or “dead,” CRISPR effector protein Cas9 that lacks endonuclease activity. This catalytic deactivation can be accomplished by introducing point mutations in the two catalytic residues (D10A and H840A) of the gene encoding Cas9. The resulting dCas9 is unable to cleave double strain DNA but retains the ability to target DNA. See Jinek et al. (2012) Science. 337(6096): 816-821. As an example, two or more catalytic domains of Cas9 (RuvC I, RuvC II, and RuvC III) may be mutated to produce a mutated Cas9 substantially lacking all DNA cleavage activity. In some embodiments, a D10A mutation is combined with one or more of H840A, N854A, or N863A mutations to produce a Cas9 enzyme substantially lacking all DNA cleavage activity. In some embodiments, a CRISPR enzyme is considered to substantially lack all DNA cleavage activity when the DNA cleavage activity of the mutated enzyme is less than about 25%, 10%, 5%, 1%, 0.1%, 0.01%, or lower with respect to its non-mutated form. Other mutations may be useful; where the Cas9 or other CRISPR enzyme is from a species other than S. pyogenes, mutations in corresponding amino acids may be made to achieve similar effects.

The terms “functional fragment” refer to a less than full-length structure that comprises the biological function of the full-length structure. In some embodiments, a functional fragment is a truncation mutant comprising a sequence that is or comprises at least 60% sequence identity to a base sequence and maintains the biological function of that full-lngth seqeunce.

The term “transcriptional modulator” refers to a molecule that has certain effect, either positive or negative, on the transcription of one or more genes or constructs. A transcriptional modulator that has a positive effect on the transcription of one or more genes or constructs, such as activating transcription thereof, is called “transcriptional activator.” Non-limiting examples of such anscriptional activators include the herpes virus VP 16 domain (Gilbert et al., Cell, 2013, 154:442-451, incorporated by reference herein), the VP64 domain which consists of a tetrameric repeat of the minimal activation domain of the VP 16 domain (Seipel, K. et al., EMBO J., 1992, 11: 4961-4968, incorporated by reference herein), and the C-terminal transcriptional activation domain of RelA (p65) NF-kappaB (nuclear factor kappaB) subunit (p65 activation domain or p65AD) (O'Shea et al., Biochem. Soc. Trans., 2008, 36 (Pt 4): 603-608 (incorporated by reference herein). Thus, in some embodiments, the transcriptional activator comprises a VP 16 domain. In some embodiments, the transcriptional activator comprises the VP64 domain. In some embodiments, the transcriptional activator comprises a plurality of VP64 domains. In some embodiments, the transcriptional activator comprises the p65 activation domain.

On the other hand, a transcriptional modulator that has a negative effect on the transcription of one or more genes or constructs, such as repressing transcription thereof, is called “transcriptional repressor.” Non-limiting examples of such transcriptional repressors include the Kruppel associated box (KRAB) repressor domain (see e.g., Margolin et al., PNAS, 1994, 91: 4509-4513, incorporated by reference herein) and the chromoshadow repressor domain (CSD) (see e.g., Lechner et al., Mol. Cell Biol., 2000, 20: 6449-6465, incorporated by reference herein). In some embodiments therefore, the transcriptional repressor comprises the KRAB repressor domain. In some embodiments therefore, the transcriptional repressor comprises the CSD repressor domain.

A non-limiting example of the nuclease-deficient sgRNA-mediated nuclease may be a synthetic dCas9-p65AD fusion protein having the following sequence:

(SEQ ID NO: 12)
MDKKYSIGLAIGTNSVGWAVITDEYKVPSKKFKVL
GNTDRHSIKKNLIGALLFDSGETAEATRLKRTARR
RYTRRKNRICYLQEIFSNEMAKVDDSFFHRLEESF
LVEEDKKHERHPIFGNIVDEVAYHEKYPTIYHLRK
KLVDSTDKADLRLIYLALAHMIKFRGHFLIEGDLN
PDNSDVDKLFIQLVQTYNQLFEENPINASGVDAKA
ILSARLSKSRRLENLIAQLPGEKKNGLFGNLIALS
LGLTPNFKSNFDLAEDAKLQLSKDTYDDDLDNLLA
QIGDQYADLFLAAKNLSDAILLSDILRVNTEITKA
PLSASMIKRYDEHHQDLTLLKALVRQQLPEKYKEI
FFDQSKNGYAGYIDGGASQEEFYKFIKPILEKMDG
TEELLVKLNREDLLRKQRTFDNGSIPHQIHLGELH
AILRRQEDFYPFLKDNREKIEKILTFRIPYYVGPL
ARGNSRFAWMTRKSEETITPWNFEEVVDKGASAQS
FIERMTNFDKNLPNEKVLPKHSLLYEYFTVYNELT
KVKYVTEGMRKPAFLSGEQKKAIVDLLFKTNRKVT
VKQLKEDYFKKIECFDSVEISGVEDRFNASLGTYH
DLLKIIKDKDFLDNEENEDILEDIVLTLTLFEDRE
MIEERLKTYAHLFDDKVMKQLKRRRYTGWGRLSRK
LINGIRDKQSGKTILDFLKSDGFANRNFMQLIHDD
SLTFKEDIQKAQVSGQGDSLHEHIANLAGSPAIKK
GILQTVKVVDELVKVMGRHKPENIVIEMARENQTT
QKGQKNSRERMKRIEEGIKELGSQILKEHPVENTQ
LQNEKLYLYYLQNGRDMYVDQELDINRLSDYDVDA
IVPQSFLKDDSIDNKVLTRSDKNRGKSDNVPSEEV
VKKMKNYWRQLLNAKLITQRKFDNLTKAERGGLSE
LDKAGFIKRQLVETRQITKHVAQILDSRMNTKYDE
NDKLIREVKVITLKSKLVSDFRKDFQFYKVREINN
YHHAHDAYLNAVVGTALIKKYPKLESEFVYGDYKV
YDVRKMIAKSEQEIGKATAKYFFYSNIMNFFKTEI
TLANGEIRKRPLIETNGETGEIVWDKGRDFATVRK
VLSMPQVNIVKKTEVQTGGFSKESILPKRNSDKLI
ARKKDWDPKKYGGFDSPTVAYSVLVVAKVEKGKSK
KLKSVKELLGITIMERSSFEKNPIDFLEAKGYKEV
KKDLIIKLPKYSLFELENGRKRMLASAGELQKGNE
LALPSKYVNFLYLASHYEKLKGSPEDNEQKQLFVE
QHKHYLDEIIEQISEFSKRVILADANLDKVLSAYN
KHRDKPIREQAENIIHLFTLTNLGAPAAFKYFDTT
IDRKRYTSTKEVLDATLIHQSITGLYETRIDLSQL
GGDEGAPKKKRKVGSSGSPKKKRKVGSQYLPDTDD
RHRIEEKRKRTYETFKSIMKKSPFSGPTDPRPPPR
RIAVPSRSSASVPKPAPQPYPFTSSLSTINYDEFP
TMVFPSGQISQASALAPAPPQVLPQAPAPAPAPAM
VSALAQAPAPVPVLAPGPPQAVAPPAPKPTQAGEG
TLSEALLQLQFDDEDLGALLGNSTDPAVFTDLASV
DNSEFQQLLNQGIPVAPHTTEPMLMEYPEAITRLV
TGAQRPPDPAPAPLGAPGLPNGLLSGDEDFSSIAD
MDFSALLSQISSGS.

Another non-limiting example of the nuclease-deficient sgRNA-mediated nuclease may be a synthetic dCAS9-VP64 fusion protein having the following sequence:

(SEQ ID NO: 13)
MDKKYSIGLAIGTNSVGWAVITDEYKVPSKKFKVL
GNTDRHSIKKNLIGALLFDSGETAEATRLKRTARR
RYTRRKNRICYLQEIFSNEMAKVDDSFFHRLEESF
LVEEDKKHERHPIFGNIVDEVAYHEKYPTIYHLRK
KLVDSTDKADLRLIYLALAHMIKFRGHFLIEGDLN
PDNSDVDKLFIQLVQTYNQLFEENPINASGVDAKA
ILSARLSKSRRLENLIAQLPGEKKNGLFGNLIALS
LGLTPNFKSNFDLAEDAKLQLSKDTYDDDLDNLLA
QIGDQYADLFLAAKNLSDAILLSDILRVNTEITKA
PLSASMIKRYDEHHQDLTLLKALVRQQLPEKYKEI
FFDQSKNGYAGYIDGGASQEEFYKFIKPILEKMDG
TEELLVKLNREDLLRKQRTFDNGSIPHQIHLGELH
AILRRQEDFYPFLKDNREKIEKILTFRIPYYVGPL
ARGNSRFAWMTRKSEETITPWNFEEVVDKGASAQS
FIERMTNFDKNLPNEKVLPKHSLLYEYFTVYNELT
KVKYVTEGMRKPAFLSGEQKKAIVDLLFKTNRKVT
VKQLKEDYFKKIECFDSVEISGVEDRFNASLGTYH
DLLKIIKDKDFLDNEENEDILEDIVLTLTLFEDRE
MIEERLKTYAHLFDDKVMKQLKRRRYTGWGRLSRK
LINGIRDKQSGKTILDFLKSDGFANRNFMQLIHDD
SLTFKEDIQKAQVSGQGDSLHEHIANLAGSPAIKK
GILQTVKVVDELVKVMGRHKPENIVIEMARENQTT
QKGQKNSRERMKRIEEGIKELGSQILKEHPVENTQ
LQNEKLYLYYLQNGRDMYVDQELDINRLSDYDVDA
IVPQSFLKDDSIDNKVLTRSDKNRGKSDNVPSEEV
VKKMKNYWRQLLNAKLITQRKFDNLTKAERGGLSE
LDKAGFIKRQLVETRQITKHVAQILDSRMNTKYDE
NDKLIREVKVITLKSKLVSDFRKDFQFYKVREINN
YHHAHDAYLNAVVGTALIKKYPKLESEFVYGDYKV
YDVRKMIAKSEQEIGKATAKYFFYSNIMNFFKTEI
TLANGEIRKRPLIETNGETGEIVWDKGRDFATVRK
VLSMPQVNIVKKTEVQTGGFSKESILPKRNSDKLI
ARKKDWDPKKYGGFDSPTVAYSVLVVAKVEKGKSK
KLKSVKELLGITIMERSSFEKNPIDFLEAKGYKEV
KKDLIIKLPKYSLFELENGRKRMLASAGELQKGNE
LALPSKYVNFLYLASHYEKLKGSPEDNEQKQLFVE
QHKHYLDEIIEQISEFSKRVILADANLDKVLSAYN
KHRDKPIREQAENIIHLFTLTNLGAPAAFKYFDTT
IDRKRYTSTKEVLDATLIHQSITGLYETRIDLSQL
GGDEGAPKKKRKVGSSGSPKKKRKVGSDALDDFDL
DMLGSDALDDFDLDMLGSDALDDFDLDMLGSDALD
DFDLDMLGSPKKKRKVGS.

A yet another non-limiting example of the nuclease-deficient sgRNA-mediated nuclease may be a synthetic dCas9-KRAB fusion protein having the following sequence:

(SEQ ID NO: 14)
MDKKYSIGLAIGTNSVGWAVITDEYKVPSKKFKVL
GNTDRHSIKKNLIGALLFDSGETAEATRLKRTARR
RYTRRKNRICYLQEIFSNEMAKVDDSFFHRLEESF
LVEEDKKHERHPIFGNIVDEVAYHEKYPTIYHLRK
KLVDSTDKADLRLIYLALAHMIKFRGHFLIEGDLN
PDNSDVDKLFIQLVQTYNQLFEENPINASGVDAKA
ILSARLSKSRRLENLIAQLPGEKKNGLFGNLIALS
LGLTPNFKSNFDLAEDAKLQLSKDTYDDDLDNLLA
QIGDQYADLFLAAKNLSDAILLSDILRVNTEITKA
PLSASMIKRYDEHHQDLTLLKALVRQQLPEKYKEI
FFDQSKNGYAGYIDGGASQEEFYKFIKPILEKMDG
TEELLVKLNREDLLRKQRTFDNGSIPHQIHLGELH
AILRRQEDFYPFLKDNREKIEKILTFRIPYYVGPL
ARGNSRFAWMTRKSEETITPWNFEEVVDKGASAQS
FIERMTNFDKNLPNEKVLPKHSLLYEYFTVYNELT
KVKYVTEGMRKPAFLSGEQKKAIVDLLFKTNRKVT
VKQLKEDYFKKIECFDSVEISGVEDRFNASLGTYH
DLLKIIKDKDFLDNEENEDILEDIVLTLTLFEDRE
MIEERLKTYAHLFDDKVMKQLKRRRYTGWGRLSRK
LINGIRDKQSGKTILDFLKSDGFANRNFMQLIHDD
SLTFKEDIQKAQVSGQGDSLHEHIANLAGSPAIKK
GILQTVKVVDELVKVMGRHKPENIVIEMARENQTT
QKGQKNSRERMKRIEEGIKELGSQILKEHPVENTQ
LQNEKLYLYYLQNGRDMYVDQELDINRLSDYDVDA
IVPQSFLKDDSIDNKVLTRSDKNRGKSDNVPSEEV
VKKMKNYWRQLLNAKLITQRKFDNLTKAERGGLSE
LDKAGFIKRQLVETRQITKHVAQILDSRMNTKYDE
NDKLIREVKVITLKSKLVSDFRKDFQFYKVREINN
YHHAHDAYLNAVVGTALIKKYPKLESEFVYGDYKV
YDVRKMIAKSEQEIGKATAKYFFYSNIMNFFKTEI
TLANGEIRKRPLIETNGETGEIVWDKGRDFATVRK
VLSMPQVNIVKKTEVQTGGFSKESILPKRNSDKLI
ARKKDWDPKKYGGFDSPTVAYSVLVVAKVEKGKSK
KLKSVKELLGITIMERSSFEKNPIDFLEAKGYKEV
KKDLIIKLPKYSLFELENGRKRMLASAGELQKGNE
LALPSKYVNFLYLASHYEKLKGSPEDNEQKQLFVE
QHKHYLDEIIEQISEFSKRVILADANLDKVLSAYN
KHRDKPIREQAENIIHLFTLTNLGAPAAFKYFDTT
IDRKRYTSTKEVLDATLIHQSITGLYETRIDLSQL
GGDAYPYDVPDYASLGSGSPKKKRKVEDPKKKRKV
DGIGSGSNGSSGSGGSGGGSMDAKSLTAWSRTLVT
FKDVFVDFTREEWKLLDTAQQIVYRNVMLENYKNL
VSLGYQLTKPDVILRLEKGEEP.

A further non-limiting example of the nuclease-deficient sgRNA-mediated nuclease may be a synthetic dCas9-CSD fusion protein having the following sequence:

(SEQ ID NO: 15)
MDKKYSIGLAIGTNSVGWAVITDEYKVPSKKFKVL
GNTDRHSIKKNLIGALLFDSGETAEATRLKRTARR
RYTRRKNRICYLQEIFSNEMAKVDDSFFHRLEESF
LVEEDKKHERHPIFGNIVDEVAYHEKYPTIYHLRK
KLVDSTDKADLRLIYLALAHMIKFRGHFLIEGDLN
PDNSDVDKLFIQLVQTYNQLFEENPINASGVDAKA
ILSARLSKSRRLENLIAQLPGEKKNGLFGNLIALS
LGLTPNFKSNFDLAEDAKLQLSKDTYDDDLDNLLA
QIGDQYADLFLAAKNLSDAILLSDILRVNTEITKA
PLSASMIKRYDEHHQDLTLLKALVRQQLPEKYKEI
FFDQSKNGYAGYIDGGASQEEFYKFIKPILEKMDG
TEELLVKLNREDLLRKQRTFDNGSIPHQIHLGELH
AILRRQEDFYPFLKDNREKIEKILTFRIPYYVGPL
ARGNSRFAWMTRKSEETITPWNFEEVVDKGASAQS
FIERMTNFDKNLPNEKVLPKHSLLYEYFTVYNELT
KVKYVTEGMRKPAFLSGEQKKAIVDLLFKTNRKVT
VKQLKEDYFKKIECFDSVEISGVEDRFNASLGTYH
DLLKlIKDKDFLDNEENEDILEDIVLTLTLFEDRE
MIEERLKTYAHLFDDKVMKQLKRRRYTGWGRLSRK
LINGIRDKQSGKTILDFLKSDGFANRNFMQLIHDD
SLTFKEDIQKAQVSGQGDSLHEHIANLAGSPAIKK
GILQTVKVVDELVKVMGRHKPENIVIEMARENQTT
QKGQKNSRERMKRIEEGIKELGSQILKEHPVENTQ
LQNEKLYLYYLQNGRDMYVDQELDINRLSDYDVDA
IVPQSFLKDDSIDNKVLTRSDKNRGKSDNVPSEEV
VKKMKNYWRQLLNAKLITQRKFDNLTKAERGGLSE
LDKAGFIKRQLVETRQITKHVAQILDSRMNTKYDE
NDKLIREVKVITLKSKLVSDFRKDFQFYKVREINN
YHHAHDAYLNAVVGTALIKKYPKLESEFVYGDYKV
YDVRKMIAKSEQEIGKATAKYFFYSNIMNFFKTEI
TLANGEIRKRPLIETNGETGEIVWDKGRDFATVRK
VLSMPQVNIVKKTEVQTGGFSKESILPKRNSDKLI
ARKKDWDPKKYGGFDSPTVAYSVLVVAKVEKGKSK
KLKSVKELLGITIMERSSFEKNPIDFLEAKGYKEV
KKDLIIKLPKYSLFELENGRKRMLASAGELQKGNE
LALPSKYVNFLYLASHYEKLKGSPEDNEQKQLFVE
QHKHYLDEIIEQISEFSKRVILADANLDKVLSAYN
KHRDKPIREQAENIIHLFTLTNLGAPAAFKYFDTT
IDRKRYTSTKEVLDATLIHQSITGLYETRIDLSQL
GGDAYPYDVPDYASLGSGSPKKKRKVEDPKKKRKV
DGIGSGSNGSSGSGGSGGGSSADDIKSKKKREQSN
DIARGFERGLEPEKIIGATDSCGDLMFLMKWKDTD
EADLVLAKEANVKCPQIVIAFYEERLTWHAYPEDA
ENKEKETAKS.

In some embodiments therefore, the nuclease-deficient sgRNA-mediated nuclease may be a synthetic dCas9-p65AD fusion protein comprising at least about 70% sequence identity to SEQ ID NO: 12. In some embodiments, the nuclease-deficient sgRNA-mediated nuclease may be a synthetic dCAS9-VP64 fusion protein comprising at least about 70% sequence identity to SEQ ID NO: 13. In some embodiments, the nuclease-deficient sgRNA-mediated nuclease may be a synthetic dCas9-KRAB fusion protein comprising at least about 70% sequence identity to SEQ ID NO: 14. In some embodiments, the nuclease-deficient sgRNA-mediated nuclease may be a synthetic dCas9-CSD fusion protein comprising at least about 70% sequence identity to SEQ ID NO: 15.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components. In some embodiments, the sample is a human tissue sample, serum, plasma, blood draw, brushing, biopsy, or surgical resection of the subject.

The term “subject” used herein refers to a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered. In some embodiments, the subject is one in need of treatment for an underlying disease or disorder, such as but not limited to glioma.

As used herein, the phrase “in need thereof” means that the animal or mammal has been identified or suspected as having a need for the particular method or treatment. In some embodiments, the identification can be by any means of diagnosis or observation. In any of the methods and treatments described herein, the animal or mammal can be in need thereof In some embodiments, the animal or mammal is in an environment or will be traveling to an environment in which a particular disorder or condition is prevalent or more likely to occur.

As used herein, the term “patient” refers to a subject afflicted with a disease or disorder. The term “patient” includes human and veterinary subjects. In some aspects of the disclosed methods, the “patient” has been diagnosed with a need for treatment for glioma, such as, for example, prior to the administering step.

“Inhibit,” “inhibiting” and “inhibition” mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in an aspect, the inhibition or reduction can be at least about 10, 20, 30, 40, 50, 60, 70, 80, 90, 100%, or any amount of reduction in between as compared to native or control levels. In an aspect, the inhibition or reduction is about 10-20, 20-30, 30-40, 40-50, 50-60, 60-70, 70-80, 80-90, or 90-100% as compared to native or control levels. In some embodiments, the inhibition or reduction is about 0-25, 25-50, 50-75, or 75-100% as compared to native or control levels.

“Modulate”, “modulating” and “modulation” as used herein mean a change in activity or function or number. The change may be an increase or a decrease, an enhancement or an inhibition of the activity, function or number.

As used herein, the term “determining” can refer to measuring or ascertaining a quantity or an amount or a change in activity. For example, determining the amount of a disclosed nucleotide in a sample as used herein can refer to the steps that the skilled person would take to measure or ascertain some quantifiable value of the nucleotide in the sample. The art is familiar with the ways to measure an amount of the disclosed nucleotides in a sample.

As used herein, the term “organoid” means an organized mass of cell types generated in vitro that mimics at least to some degree the structure, marker expression, or function of a naturally occurring organ. A “human brain organoid” is an in vitro generated body that mimics structure and function of a human brain. Human brain organoids may contain several types of nerve cells and have anatomical features that resemble human brains.

The term “therapeutically effective amount” means a quantity sufficient to achieve a desired therapeutic effect, for example, an amount which results in the prevention or amelioration of or a decrease in the symptoms associated with a disease that is being treated, e.g., disorders associated with cancer growth or a hyperproliferative disorder. The amount of compound administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The regimen of administration can affect what constitutes an effective amount. Further, several divided dosages, as well as staggered dosages, can be administered daily or sequentially, or the dose can be continuously infused, or can be a bolus injection. Further, the dosages of the compound(s) of the invention can be proportionally increased or decreased as indicated by the exigencies of the therapeutic or prophylactic situation. Typically, an effective amount of the compounds of the present invention, sufficient for achieving a therapeutic effect, range from about 0.000001 mg per kilogram body weight per day to about 10,000 mg per kilogram body weight per day. Preferably, the dosage ranges are from about 0.0001 mg per kilogram body weight per day to about 100 mg per kilogram body weight per day. The compounds disclosed herein can also be administered in combination with each other, or with one or more additional therapeutic compounds.

The terms “treating” or “treatment” or “treat” as used herein refer to both 1) therapeutic measures that cure, slow down, lessen symptoms of, and/or halt progression of a diagnosed pathologic condition or disorder, and 2) prophylactic or preventative measures that prevent or slow the development of a targeted pathologic condition or disorder. Thus, those in need of treatment include those already diagnosed with the disorder; those prone to have the disorder; and those in whom the disorder is to be prevented. In some embodiments, a subject is successfully “treated” according to the methods of the present disclosure if the patient shows one or more of the following: a reduction in the number of and/or complete absence of cancer cells; a reduction in the tumor size; an inhibition of tumor growth; inhibition of and/or an absence of cancer cell infiltration into peripheral organs including the spread of cancer cells into soft tissue and bone; inhibition of and/or an absence of tumor or cancer cell metastasis; inhibition and/or an absence of cancer growth; relief of one or more symptoms associated with the specific cancer; reduced morbidity and mortality; improvement in quality of life; reduction in tumorigenicity; reduction in the number or frequency of cancer stem cells; or some combination of such effects.

The term “tumor” as used herein, refers to all neoplastic cell growth and proliferation, whether malignant or benign, and all pre-cancerous and cancerous cells and tissues.

The term “glioma” refers to a type of tumor that occurs in the brain and spinal cord. Gliomas begin in the gluey supportive cells (glial cells) that surround nerve cells and help them function. Three types of glial cells can produce tumors and gliomas are classified according to the type of glial cell involved in the tumor, as well as the tumor's genetic features, which can help predict how the tumor will behave over time and the treatments most likely to work. Types of glioma include astrocytomas (including astrocytoma, anaplastic astrocytoma and glioblastoma), ependymomas (including anaplastic ependymoma, myxopapillary ependymoma and subependymoma), and oligodendrogliomas (including oligodendroglioma, anaplastic oligodendroglioma and anaplastic oligoastrocytoma).

The term “malignant glioma” refers to a group of gliomas consisting of glioblastomas, anaplastic astrocytomas, anaplastic oligodendrogliomas, and anaplastic oligoastrocytomas, and some less common tumors such as anaplastic ependymomas and anaplastic gangliogliomas.

### Method for Identification of a Radiotherapy Sensitizer

The recently developed CRISPRi technology, or clustered regularly interspaced short palindromic repeats interference, a technology that can repress transcription of any gene via the targeted recruitment of the nuclease-dead dCas9-KRAB repressor fusion protein to the transcription start site (TSS) by a single guide RNA (see Qi et al. (2013) Cell. 152 (5): 1173-1183, incorporated by reference herein), makes genome-scale CRISPRi screening of lncRNA gene function possible. See Liu et al. (2017) Science. 355:eaah7111 (incorporated by reference herein) and US 2017/0204407 (incorporated by reference herein). This technology is adapted and modified in the present disclosure for identification of certain lncRNAs as radiotherapy sensitizers.

Generally, radiotherapy sensitizers may be obtained by exposing a plurality of test cells to a radiation and selecting a test cell that has decreased cell proliferation as compared to a control cell treated with the radiation alone. Each test cell comprises a small guide RNA (sgRNA) that targets a locus encoding a lncRNA and a nuclease-deficient sgRNA-mediated nuclease (dCas9) and thus, the locus targeted by the sgRNA comprised in the test cell selected can be then identified as a radiotherapy sensitizer. The plurality of test cells for such a screening may be obtained by using a CRISPRi library targeting lncRNA loci, or CRiNCL, as described in, for example, Liu et al. (2017, Science. 355:eaah7111; incorporated by reference in its entirty herein), and the cells may be engineered to stably express the dCas9. In some embodiments, the dCas9 comprises a dCas9 domain fused to a transcriptional modulator, which may be a transcriptional activator or a transcriptional repressor. Any known transcriptional activators or transcriptional repressors can be used.

In some embodiments, the dCas9 is a transcriptional activator and comprises a dCas9 domain and a transcriptional activator domain. In some embodiments, the dCas9 domain is fused to a p65 activation domain (p65AD) as exemplified in SEQ ID NO: 12. In some embodiments, the dCas9 domain is fused to one or more copies of a VP8, VP16 or VP64 activation domain as exemplified in SEQ ID NO: 13, which contains a dCas9 domain fused to VP16 or VP64. Both SEQ ID NO: 12 and SEQ ID NO: 13 are two non-limiting, representative examples of such dCas9. In some embodiments, the dCas9 domain fused to a p65AD is at least about 70%, 75%, 80%, 85%, 90%, 95%, 96%, 97%, 98%, or 99% identical, or 100% identical, to SEQ ID NO: 12. In some embodiments, the dCas9 domain fused to a VP8, VP16 or VP64 activation domain is at least about 70%, 75%, 80%, 85%, 90%, 95%, 96%, 97%, 98%, or 99% identical, or 100% identical, to SEQ ID NO: 13.

In some embodiments, the dCas9 is a transcriptional repressor and comprises a dCas9 domain and a transcriptional repressor domain. In some embodiments, the dCas9 domain is fused to a Kruppel associated box (KRAB) repressor domain as exemplified in SEQ ID NO: 14. In some embodiments, the dCas9 domain is fused to a chromoshadow repressor domain as exemplified in SEQ ID NO: 15. Both SEQ ID NO: 14 and SEQ ID NO: 15 are two non-limiting, representative examples of such dCas9. In some embodiments, the dCas9 domain fused to a KRAB repressor domain is at least about 70%, 75%, 80%, 85%, 90%, 95%, 96%, 97%, 98%, or 99% identical, or 100% identical, to SEQ ID NO: 14. In some embodiments, the dCas9 domain fused to a chromoshadow repressor domain is at least about 70%, 75%, 80%, 85%, 90%, 95%, 96%, 97%, 98%, or 99% identical, or 100% identical, to SEQ ID NO: 15.

Any type of cells may be used and non-limiting examples of suitable cells may include K562, U87, HeLa, HEK293T, MCF7, MDA-MB-231, and iPSC. In some embodiments, the cells used are U87 cells, which is a human primary glioblastoma cell line that is commonly used in brain cancer research. In some embodiments, the cells used in the method of the present disclosure are primary glioblastomas (GBM) cells. In some embodiments, the cells used are primary diffuse intrinsic pontine gliomas (DIPG) cells.

Any type of radiation, either ionizing or non-ionizing, can be used to expose the cells. Ionizing radiation includes, but not limited to, gamma rays, X-rays, and the higher ultraviolet part of the electromagnetic spectrum, whereas non-ionizing radiation includes, but not limited to, the lower ultraviolet part of the electromagnetic spectrum and all the spectrum below UV, including visible light (including nearly all types of laser light), infrared, microwaves, and radio waves. In some embodiments, the radiation used to expose the cells is ionizing radiation. In some embodiments, the radiation used to expose the cells is gamma rays.

The radiation used to expose the cells should be in a dose that is clinically relevant, either in a single fraction or in several fractions. In some embodiments, the radiation used to expose the cells is at a total dose of from about 1 Gy to about 12 Gy, from about 2 Gy to about 10 Gy, from about 3 Gy to about 8 Gy, or from about 4 Gy to about 6 Gy. In some embodiments, the radiation is delivered in about 1 to about 6 fractions, for example, in about 1, 2, 3, 4, 5 or 6 fractions. In some embodiments, the radiation used to expose the cells is at a total dose of about 8 Gy and delivered in about 4 fractions.

After radiation treatment, the test cells are selected on the basis of the phenotype comprising culturing the cells, and thereby selecting the cells on the basis of cellular proliferation as compared to a control cell treated with the radiation alone. Suitable control cells include, but not limited to, the same cell type from which the test cells are obtained and may comprise the same dCas9 but without a sgRNA.

The locus targeted by the sgRNA in a test cell thus selected, or so-called a radiotherapy sensitizer, may be identified using any method known in the art, including, but not limited to, RNA-seq analysis. In some embodiments, the radiotherapy sensitizer identified by the method discloed herein is specific to glioma and thus also called lncRNA Glioma Radiation Sensitizers (lncGRS). In some embodiments, the lncGRS identified by the method disclosed herein comprises a identifying a nucleic acid sequence having at least about 70%, 75%, 80%, 85%, 90%, 95%, 96%, 97%, 98%, or 99% sequence identity to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 and SEQ ID NO: 9 by exposure to an agent that binds or non-covalently binds to a nucleic acid sequence having at least about 70%, 75%, 80%, 85%, 90%, 95%, 96%, 97%, 98%, or 99% sequence identity to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 and SEQ ID NO: 9. In some embodiments, the lncGRS identified by the method discloed herein comprises the nucleic acid sequence of any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 and SEQ ID NO: 9.

The disclosure relates to a method of preparing RNA from a sample comprising digesting a sample to obtain an amount of total RNA; isolating the RNA with a reagent specific to bind or associate to single-stranded RNA; centrifuging the isolated RNA to separate the RNA from other particulate matter and then purifying the RNA from the sample. In some embodiments, the method further comprises analyzing the isolated RNA with one or a plurality of compositions disclosed herein that is capable of detecting the presence, absence or quantity of specific RNA sequences in the sample. In some embodiments, RNA isolation from a sample is performed by subcellular fractionation of cells obtained form a sample. In some embodiments, the sample is one or a plurality of cells from a tumor, optionally suspected of being a malignant glioma. In some embodiments, the sample is one or a plurality of cells from a malignant glioma. In some embodiments, the step of analyzing the sample of isolated RNA comprises detecting the presence, absence or quantity of lncRNAs in a sample that comprise at least 70% sequence identity to one or a plurality of RNAs in Table 1. In some embodiments, the detecting step is performed by detecting a binding event between lncRNAs in a sample that comprise at least 70% sequence identity to one or a plurality of RNAs in Table 1 and a nucleic acid sequence that comprises at least 70% sequence identity to a sequence complementary to one or a plurality of RNAs in Table 1 (e.g. at least 70% sequence identity to SEQ ID NO: 38, SEQ ID NO:39, SEQ ID NO:40, SEQ ID NO:41, SEQ ID NO:42, SEQ ID NO:43, SEQ ID NO:45, or SEQ ID NO:46 or functional fragment thereof).

In some embodiments, the methods of the disclosure relate to performing nuclease-based modification of cells to identify lncRNAs associated with glioma. In some embodiments, the methods of the disclosure relate to performing nuclease-based modification of cells to identify lncRNAs from glioma tumors. In some embodiments, the methods of the disclosure relate to performing nuclease-based modification of cells to identify lncRNAs from a sample of malignant brain cancer.

In some embodiments, the cells from which samples are obtained to produce an exposure or production step in the disclosed methods are from a primary culture.

The disclosure relates to a method of identifying an lncRNA associated with radiotherapy sensitivity of a patient, wherein the patient is diagnosed with or suspected of having malignant glioma. In some embodiments, the disclosure relates to a method of identifying a biomarker capable of determining whether a subject in need of treatment has a likelihood of responding to radiotherapy. In some embodiments the methods of the disclosure comprise a step of detecting the presence of any one or more lncRNAs disclosed in Table 1 and then treating the subject with a therapeutically effective amount of radiotherapy. In some embodiments, the step of treating the subject further comprises treating the subject with a therapeutically effective amount of an agent that interferes with lncRNA function of one or more of the lncRNAs in Table 1.

In some embodiment, methods peformed herein comprise the step of correlating the presence or quantity of one or a plurality of lncRNAs in a smaple from a subject to the presence of malignant glioma. In some embodiment, methods peformed herein comprise the step of correlating the presence or quantity of one or a plurality of lncRNAs in a sample from a subject to the likelihood that the subject will respond to radiotherapy, optionally with one or more agent that interfere with the function of the lncRNAs.

Methods of identifying biomarkers of sample from a subject having a brain cancer can be performed by performing steps of testing cells from the sample for the presence of radiotherapy sensitivy as performed by the methods disclosed in CRISPRi-based genome-scale identification of functional long non-coding RNA loci in human cells, Liu et al. Science. 2017 Jan. 6; 355(6320), which is incorporated by reference in its entirety.

TABLE 1
Exemplary lncGRSs identified by the
method disclosed herein.

lncGRS-l (CTC-338M12.4)
cggccgcctttcccgttgggcacccagctgtggct
gtgccctgtcctcgcccagctcccggcataggcat
gcgctctgggccggggccggggccccccgcctccc
cgggaggaatcgctgggttgactctgggtcagcaa
agggagcccggccctttctagcggggggacgtcct
gcagccgctggaggaggctgagtcacgcttttccc
accagaagtctacctgtgcctgcgcccgccggacc
tgccggcctcttccttctcatccagggggcctctt
ctccagcctcacggaggtgggaagcttgctggagg
cccctacctgccttttggggaagaccccgaggctg
gcccacgccctgctggcaggacccttcctgactgg
gtccaccgttcttctgggtctcaccacttctccca
gcccctgggctgcttcctctctcccgtgtctggga
ccccagaggcaccacttccattccatacaagcttg
cagagtcaagacgaactctaactcatgggatggac
aaactggaagatgtaaaataagtaaggctttctgg
gccaaaaagcctcttcttacagaaaatcaaatttt
aaaagaacattgacctcaaaacaataaaactgtcc
tggttatgcaatagaaatagctatataaatggaat
catatccttaatgaacacctcctgtggctacccaa
cttttcacacatcttactgattataggccttgctg
tgaatgctttcttgctctatctgaagtattcccac
cagagttgtttccaactttatttcctatattatga
aaaatgaagtttttttaacattgtaacaaagtata
tgaaagataatgtatactgtattaaataaaccatt
atacaaaaata (SEQ ID NO: 1)
lncGRS-2 (AC005624.2)
ctgggacttggagtccagtccctgggggtcccgcg
gggatggctctgggcacccgaagcccctccccagc
gccgagacaggagggctggggtagattcgagaccc
cggtttgcagagcgcaagactgaggcccagggccg
cgcagtcaggcaggaaacggcgctcctggcggatg
gaacagcacgggcaaaggctgggtatggggggtct
cgctccgttgcccacactggagtgcagtggcgcaa
tcatggctgactgcagacttgaactcctggatgca
agccattctcctgcctcagcctcccgaggagctgg
gactacaggtgtgtgccagcacatgtggctaatta
ttaaaaatttttttggctggg (SEQ ID NO: 2)
lncGRS-3 (PDXDC2P)
ctcaaccatcaggttcggcagccggcggcgccgcc
tggcagctcctcctcttctccgccccgctggccgc
gggcgcgggggacgtcagcgctgccagcgtggaaa
gagctgcggggcgcggga
ggaggaagtagagcccgggaccgccgggccaccac
cggccgcctcagccatggacgcgtccctggagaag
atagcagaccccacgttagctgaaatgggaaaaaa
cttgaaggaggcagtgaagatgctggaggacagtc
agagaagaacagaagaggaaaatggaaagaagctc
atatccagagatattccaggcccactccagggcag
tggacaagatatggtgagcatcctccagttagttc
agaatctgatgcatggagatgaagatgaggagccc
cagagccccagaatccaaaatattggagaacaagg
tcatgtggctgtgttgggacatagtctgggagctt
atattttgactctggacgaagagaagctgagaaaa
cttacaactaggatactttcagataccaccttatg
gctatgcagaattttcagatatgaaaatgggtgtg
cttatttccacgaagaggaaagagaaggacttgca
aagatatgtaggcttgccattcattctcaatatga
agacttcgtagtggatggcttcagtgggttatata
acaagaagcctgtcatatatcttagtgctgctgct
agacctggcctgggccaatacctttgtaatcagct
cggcttgcccttcccctgcttgtgccgtgtaccct
gtaacactatgtttggatcccagcatcagatggat
gttgccttcctggagaaactgattaaagatgatat
agagcgaggaagactgcccctgttgcttgtcgcaa
atgcaggaacggcagcagtaggacacacagacaag
attgggagattgaaagaactctgtgagcagtatgg
catatggcttcatgtggagggtgtgaatctggcaa
cattggctctgggttatgtctcctcatcagtgctg
gctgcagccaaatgtgatagcatgacgatgactcc
tggcccgtggctgggtttgccagctgttcctgcgg
tgacactgtataaacacgaccctgccttgacttta
gttgctggtcttatatcaaataagcccacagacaa
actccgtgccctgcctctgtggttatctttacaat
acttgggacttgatgggtttgtggagaggatcaag
catgcctgtcaactgagtcaatggttgcaggaaag
tttgaagaaagtgaattacatcaaaatcttggtgg
aagatgagctcagctccccagtggtggtgttcaga
tttttccaagaattaccaggctcagatccagtgtt
taaagccgtcccagtgcccaacatgacaccttcag
cagtcggccgggagaggcactcgtgtgatgcgctg
aatctctggctgggagaacagctgaagcagctggt
gcctgcgagcggcctcacagtcatggatctggaag
ctgagggcacgtgtttgcggttcagccctttgatg
accgcagcaggtatgatctcgtgaaaccttgagag
aaactgaatgaggaatgaaactattgttcctgttt
cacacagaagaaaactgaggttggcactcatgatg
agcccctgttctcattctgcaaatggtgaagctct
ctatcgtcctgaccccacagttcctgtcccatgac
cagggcccgctcaccaaggagctgcagcagcacgt
aaagtcagtgacatgcccatgcgagtacctgagga
aggtgaccttgaggaaccctgggagctcaggaagg
aaggagcgcccagaagcagggacagggagctggtt
ggggaggaccagaaatcaggtgcccaacagccccc
ttctcctcctttcccttcccttacttccccccttc
ccctccccttcccctccccctccccaactcagatc
tggccccggtcccatccccttccctcccccctgcc
ctaagccacctccacctctgtcctggctgcctcag
ggcgccctgaaaggaccaggacatgcgggtgcggt
ggctgcgcttttggctcctcttctggctcctgctg
ggatttatcagccatcagcccacccctgttatcaa
tactctggctgaccatcgtcatcgtgagactgact
ttggtggaagtccttggataattatcattattgtg
tttctgggacgttacaaatttaccattctcttctg
cacaatttacctttgtgtgcctttcctgaagacta
tcttctggtctcgaaatggacatgatagatccagg
gatgtacagcagagagctaggaggtccaaccgccg
tagacaggaaggaattaaaattggcctggaagaca
tctgtactttatggaaacaggcggaaacaaaagtt
caagctaaaatccgtaagatgaaggtcacaaagaa
agtcaaccatcattacaaaatcaatggaaagagga
agaccgccaaagaacaatcaccccctctgcaagaa
agcctctttgcaactggctctgtgctcaagccctg
cagagggagatggcagagaggaaggctgcctacaa
gcatcacagtcccatccctgttggtaaccgtgttg
tgcaaaaacaccttcatccccacccagtggggccc
ctgatctaatattcaaagtgtcagaggttccatat
ttgtaatagcaaatgggccctgactgtaaattagt
gaagagtgaatgtaacttattacccacagggacaa
ttccaaatgaaggccttaaatgatgctcagctaag
ctggttcttgtgtggcctctgtaccttcaaaagct
gccgagtcctatgattacacgtgatgggacttgta
cacttgaagtgaaacagttttaaaacttgctttgt
ttagaattcccacctcatttttccatggacaaaag
tattctttatgtcctagtgcacttacaatttggta
ttacctgggagtgaaaagaaatattacagccatgc
ctaactgacttcttgaggtaagattgttctgtcag
aaaaccctctcccagttcccctgcagctcttcagg
aatccacatctctgcagagctctttgttctcatgg
gtggcacctccagagtgaagaagatcctttatcaa
gaagggaaacaggggaaatgagagggtcctgcagg
cagagctggaatcaacttccactctgcctcttgca
agctgtgtgaccccgggcacaatttctccttcctc
tggaaacctctgttttcttagatttggagcagggt
ggtcacactgaccttgcagagttccgagaatcaga
gacagaacataaaaggcctggaaaacattctccaa
aaagaagctgcaacatgtgtggacaatgggctttt
catgcctctcttactgtctgttgacctggtgcaag
aaacatgctctggtgatggctgtgagggaggaatg
aggatagacatagacactcctgtgtctcaaacatg
cctctttattactctgttatgactctgtcttccct
ggggcaggaccccagcctgcctacatttgcagaca
gacacagtggcatgtggagacaacagtgtgtccca
atgacttttctttactccccagctgtcggcagtac
tcagtggaagggtgatattatgacactgatactgc
tattttgaaacctggaggatggaaaggtgcaaaaa
tctatcaccagcaacagaaggtcagaatggcggca
gcggagcatcgtcattcttcaggattgccctactg
gccctacctcacagctgaaactttaaaaaacagga
tgggccgccagccacctcctccaactcaacaacat
tctataactgataactccctgagcctcaagacacc
tcccgaatgtcttcttattccccttccaccctctc
ctcttccaccctccgtggatgataatctcaagact
cctcccttagctactcaggaggccgaggcagaaaa
atcactgaaacccaagaggcagaggttgagtaagc
tgagaacaggtcattgcactcaagcctgggcaata
agaagaaatctgtgagtggaacaaaagaaaaaaat
caaaaaacaaaacaaaacccacactccaaaaacaa
actaacaaagaataaataaataatataaaaataaa
aaaaaaaaaaaaaaaaaaaaaaaaaaaaaaaaaaa
aaaaaaaaaaaaaaaaaaaaaaaaaaaaaaaaaaa
aaaaaaaaaaaaaaaaaaaaaaaaaaaaaaaaaaa
aaaaaaaaaa (SEQ ID NO: 3)
lncGRS-4 (RP11-195F19.9)
gggctccgaacgtctcctggggagcggcatgattg
aatcagggattccctagccccaggtccccttctcc
gtcattctcaggacgaggctgcccagttctcagaa
aaatgtgaaaagagagactggcctagcctcccagc
ctacatctttctcccatcctggatgtttcctgccc
ttgaatgttggactccaagttcttcagttttggaa
ctcggactgtctctccttgctcctcagcctgcaga
cggcctattgtgggaccttgtgatcgtgttgttgt
ggtggtattactactactcagcaagattctggttt
tacttctcatcttgggagttaccatcaagacgtta
ggtatggctgtgtgacccactttggccaatgaaat
gtgaacagaaatggcatatgtcacttgtggacaaa
aacatttaagcaagaatttcccttgctgtcttcct
tgactggagcgaccatggaagcctgtgttgagatg
aagaggctgccaacagatcaaaacacttgaaaact
tgagcagccacatggactacaactgtgctggagac
ttgcctggacccacagcaggcttggtatgcatggg
gaatagaattttgttgtattacactcctcagattt
ggatgtcatttgttattacagcattacctagccta
(SEQ ID NO: 4)
lncGRS-5 (SNHG1)
ctgcctggccagggcgactggcggataaggtcttg
tgcgtggcctcgaggcttaaaagtagcagtggggc
tttgtgaaggacaaaatggcgatggcgggccgtgt
aggtcccccttcctatgatgaggaccttttcacag
acctgtactgagctccgtgaggataaataactctg
aggagatgggccctgcaagcctcttgcttagccgt
ctgttcagaaaatagcgttttcgaaatgccctgag
ttgacctaatgtcttattgggctcctgtctgcagg
atttacgcgcacgttggaaccgaagagagctctgt
tgttgcaatgttcagcccacaagagcttactggtg
aaggaatgggacaagacccatctttatgcaaagcc
agcgttacagtaatgttccagcatctcataatcta
tcctggggaattcagctgcctcccagggtgaatac
aggtattcctgatgacagtctgcctctatcttaca
gagcagcttgttgctatataccattgaaaagcctt
cagagctgagaggtactactaaccaataacctgct
tggctcaaagggccagcaccttctctctaaagccc
aagaggagtttgaggaaaactaggtgtctgtgttc
actccaggctgaagttacaggtctgagcaaataag
gtgtataaaaaatggaatctgtcttggaggacatc
agaaggtgaattttccaagttcttggacaacctag
ctgttgaaaagctttctgggtttggggggtatttc
agatgtaccttaaagtgttagcagacacagattaa
gacactgggagccaatgaaacagcagttgagggtt
tgctgtgtatcacatttctgtattttatcaccccc
ttcctgcaacattatttatctggaatctacctgcc
cttttgttttttagatacaagggcttggttttgtt
acccaggctggtttcaaggccatagctttaagaga
tcctctcaccacagatttccaaagtgctgggattg
caggtgtgattcatggcacccagactttgctgcct
ttcttacatgatccaggcccagaacccaaactcag
gcactgtatagatgaccactttcgtaaactactga
cctagcttgttgccaattgttgattgaacttccca
taactccacttcgtgtctgttcctctgtatacagc
caccttctgttcccgtcatgagcctttaggtctcc
atttgcatattgcaaatactatgttccatgtaggt
agctcattcagggccttgctcttcacttcaaaaaa
ggttcccttgaggactggctgtcaatttgtgttgc
tgtgttggttgttgatgaaaataataaaatgattg
attacata (SEQ ID NO: 5)
lncGRS-6 (RP1-122P22.2)
cccttccctcccctcctcctctttcctctcctctc
ccctcccctcccctcctttctcccccttcctcctc
ttcctcctcctcctcttgctcctcccgggcgggga
aacccagccccgcgctcgtctttggggccaccggt
cgcccccgcctccgggacctgcggggagggcccgc
gagagatggagacgagagatctggacttctgaaca
tctaccggaaaagataaaatgtgcaagcccttcag
aacgctggggaggttgggctgaactgcagtgggag
aggagaagagatgctggcgtgaagggctggggcag
aagatcaggggaaaattgcggatgatctgaatgat
agcagtgaccaccagcaacctacagagctgcccaa
ccaggaaattgtcagatggaagcaacgctt
(SEQ ID NO: 6)
lncGRS-7 (MIR210HG)
gagagggtgccagcggccgcagctgaagttgggcc
gagagccggcgacggccccgcgccggggtcgcagg
cctgcaggagttgagggctgcacctgctcgctgga
gagggagaggcagatttagtggacgcctggcatgg
actcggactggcctttggaagctccctgccctgac
ggggttgcctgtcaccactgcgaagtgaggcttgg
caggacctgcacctgagaaaggctgtgtgtggtct
tggggtccacacctgcagagctaacttactgccag
acggcgacttactgtgggccaccctcagtgaaccg
gggtgtcctcagctggccctacagagcacttctgt
gctggggatgagtaggaactctgggcgaggagggt
cccagcgccgcccctcgatacagcctggctctgcc
ctctgcccgtacttacaccaggtgggatccctgcc
ctgcattgcctggggattggctgggcttgggcccg
ccctgctgtggaactggatgttttcagggagccca
gcctttcctcatgtcaacacagttcacaatatagt
tttcaaagtacagtttaaaactcaaaagtaaactt
ttcagcaactcaaaggtttgctgagtgatctgaag
cactctggccactttttggggccatgggatttggt
tcacctgaaacagccagtgagaggccgggtgtggt
ggctcacacccgtaatcccaacacttcaggaggca
gacgcgggtgatcgctcacttgagatcaggagttc
aagaccagcctgggcaacatggtgaaacctcgtct
ctactaaaaatacaaaaattagctaggcatggtgg
tgggcacctgtaatcccagctacttggaaggctga
ggcaagagaatcgcttgaacctgggaggtggaggt
tgcagcgagacgagattacgccgctgcactccagc
ctgggtgacgagagactctgcctcaaaaaaataaa
aaaatgaaacagccagtgaggaggaaggctccccg
ccttccccccgccggaacatagccatagctgctgc
tgggacaccctcttggtggggaagaaggctggtta
gcttcatcagagccagcagcagcagaccagggacg
ggcacctaggcagtggcctcagagtgaacaggagt
tcctcagaaacacacacagggacggcgtggcgcat
gctctgccagctccatgcctccttcccattgtggg
gctggggtacgtagggcagagctcatgacctccgg
gaggacatgggggtgggctctggatggcacctggc
attgccccctgctggcctatgtgacggtgtggagg
gctggtcacagaggtacgaccatccctccagaatg
tgggtcggggctgtggatggaggagtaggcccctc
atatcccaggcctgctgcccaggcacaacccactt
ggcctatgcattccaggctccatcccatgtgactc
tgggcttagccccttctggggccacaggtcaggca
ggtccaggccccaaggacctcccagtgacaggcga
ctgtgagctgggcagacaggagtgaagtcaggtgg
gggttctggcttgctgacaccagcgtttggagcct
cctgctgctgcctggcttccctgcattccctgttc
cctgcctcaggcaagaaataaccaagccgagttgc
ctctgcacagcagtgagctcctggtggccctggct
tctggggagccctgtggatggcttccttgcccaag
tccaggccttcttgttccctttgtgtgctccagag
aaagggggcagcaccagatccagatccagggccaa
ccaacagaaagctgagtccatcccaaactcgccca
ttctcagagcacaaagaccccatgatctagggcaa
acttgtccaactgttggcccatggaacagctttga
atgcagcccaacacaaatctataaattttcttaaa
cattg (SEQ ID NO: 7)
lncGRS-8 (SNHG12)
cccggtgtcgacttactagctgcaagcctctgcct
gccttcctgcgcgccgttccccgctagtcgctgct
gctggcgcgcactcgccgggtttttcctcccacgg
cctcgagatggtggtgaatgtggcacggaggagcc
gggccttccaacccggtgggcccgagctccgaaag
gccccctcggcagtgagaggggcgggagcccgcgg
gggccgcgcccttctctcgcttcggactgcgcaac
gctgcgctctgggctgacaggcggataaaacggtc
ccatcaagactgagaaaaagcacaccagctattgg
cacagcgtgggcagtggggcctacaggatgactga
cttagtctacagagatcccggcgtacttaagcaga
tgaagactcttaagatgacagaaggtgatttttct
ggtgatcgaggacttccggggtaatgacagtgatg
aaatgcaggggacctggttgcccccaagtttcctg
gcagtgtgtgatactgaggaggtgagcttgtttct
ggagctgtgctttaagattcatgttacatgtaaag
ctgtcctcatttgtgactatggacctatggagttg
ggacaatctctatgggaagcagaaggcaaggaccc
cggtcattttaggtagaaacaacagcatgctaatg
caaaaaattatgcagtgtgctactgaacttcagag
gtgatcaataaaagaagaataaaaagactaataaa
agtaaaaaaaaaaaaaaaaaaaa
(SEQ ID NO: 8)
lncGRS-9 (RP11-339B21.10)
gggctcagggaccagagcgggattatggaaatcaa
aaccaagcttgcagtggaggggagaggtatgggcc
actttgagaggtgcctcgtgtgggagggggtcaga
atggacctcaccattgaggggccgggagaagaggc
ttctaggcagaagagaacagtgggtgccaaggccc
tgaggcaggaaggtgtttaggagcagaaagaagga
agcctggggcttagcatgtgctcagtacttggcca
agaagcctcggcccaggggctgggagtgtgagcgc
aggaaagaggttgctggacacggcaggagagaatg
ggaactgggccctccaagggcagcccttccaggag
atgggacatggattccttgtttgtgttctctgcta
tagccaaatatatgtgcacgtgta
((SEQ ID NO: 9)

### Methods of Diagnosis and Treatment

In one aspect, the disclosure provides a method of diagnosing malignant glioma in a subject comprising detecting the presence, absence or quantity of any one of the lncGRSs disclosed herein in the subject. The presence or absence of any one of the lncGRSs disclosed herein in the subject may be detected using any method known in the art and a positive detection of the presence of any one of the lncGRSs disclosed herein in the subject is indicative of said subject having malignant glioma. In some embodiments, the presence or absence of any one of the lncGRSs disclosed herein in the subject is detected by using a nucleic acid that is complementary, or at least substantially complementary, to any one of the lncGRSs disclosed herein and detecting the interaction between them. Such interaction may be detected by way of, for instance, fluorescence, radioactivity, enzyme-linked electrochemical, or chemoluminescence, all of which are well known and routinely used by one skilled in the art. In some embodiments, the lncGRS disclosed herein comprises a nucleic acid sequence having at least about 70%, 75%, 80%, 85%, 90%, 95%, 96%, 97%, 98%, 99% or 100% sequence identity to SEQ ID NO: 1. In some embodiments, the lncGRS disclosed herein comprises a nucleic acid sequence having at least about 70%, 75%, 80%, 85%, 90%, 95%, 96%, 97%, 98%, 99% or 100% sequence identity to SEQ ID NO: 2. In some embodiments, the lncGRS disclosed herein comprises a nucleic acid sequence having at least about 70%, 75%, 80%, 85%, 90%, 95%, 96%, 97%, 98%, 99% or 100% sequence identity to SEQ ID NO: 3. In some embodiments, the lncGRS disclosed herein comprises a nucleic acid sequence having at least about 70%, 75%, 80%, 85%, 90%, 95%, 96%, 97%, 98%, 99% or 100% sequence identity to SEQ ID NO: 4. In some embodiments, the lncGRS disclosed herein comprises a nucleic acid sequence having at least about 70%, 75%, 80%, 85%, 90%, 95%, 96%, 97%, 98%, 99% or 100% sequence identity to SEQ ID NO: 5. In some embodiments, the lncGRS disclosed herein comprises a nucleic acid sequence having at least about 70%, 75%, 80%, 85%, 90%, 95%, 96%, 97%, 98%, 99% or 100% sequence identity to SEQ ID NO: 6. In some embodiments, the lncGRS disclosed herein comprises a nucleic acid sequence having at least about 70%, 75%, 80%, 85%, 90%, 95%, 96%, 97%, 98%, 99% or 100% sequence identity to SEQ ID NO: 7. In some embodiments, the lncGRS disclosed herein comprises a nucleic acid sequence having at least about 70%, 75%, 80%, 85%, 90%, 95%, 96%, 97%, 98%, 99% or 100% sequence identity to SEQ ID NO: 8. In some embodiments, the lncGRS disclosed herein comprises a nucleic acid sequence having at least about 70%, 75%, 80%, 85%, 90%, 95%, 96%, 97%, 98%, 99% or 100% sequence identity to SEQ ID NO: 9. In some embodiments, the malignant glioma is glioblastoma or diffuse intrinsic pontine gliomas.

Nucleic acids that are complementary, or at least substantially complementary, to any one of the lncGRSs disclosed herein can be identified using any methods known in the art. Non-limiting examples are provided below, which are the nucleotide sequences that are fully completary to the lncGRS disclosed herein. In some embodiments, the method of diagnosing a subject with malignant glioma or radiosensitive malignant glioma comprise a step of detecting the presence, absence, or quantity of lncRNAs in a sample by detecting hybridization of the lncRNAs to a nucleic acid sequence complementary to the lncRNAs. In some embodiments, that nucleic acid sequence is a cDNA or RNA or hybrid DNA/RNA molecule optionally immbolized to a ssolid substrate.

Complementary sequence to IncGRS-l
(CTC-338M12.4)
(SEQ ID NO: 38)
tatttttgtataatggtttatttaatacagtatac
attatctttcatatactttgttacaatgttaaaaa
aacttcatttttcataatataggaaataaagttgg
aaacaactctggtgggaatacttcagatagagcaa
gaaagcattcacagcaaggcctataatcagtaaga
tgtgtgaaaagttgggtagccacaggaggtgttca
ttaaggatatgattccatttatatagctatttcta
ttgcataaccaggacagttttattgttttgaggtc
aatgttcttttaaaatttgattttctgtaagaaga
ggctttttggcccagaaagccttacttattttaca
tcttccagtttgtccatcccatgagttagagttcg
tcttgactctgcaagcttgtatggaatggaagtgg
tgcctctggggtcccagacacgggagagaggaagc
agcccaggggctgggagaagtggtgagacccagaa
gaacggtggacccagtcaggaagggtcctgccagc
agggcgtgggccagcctcggggtcttccccaaaag
gcaggtaggggcctccagcaagcttcccacctccg
tgaggctggagaagaggccccctggatgagaagga
agaggccggcaggtccggcgggcgcaggcacaggt
agacttctggtgggaaaagcgtgactcagcctcct
ccagcggctgcaggacgtccccccgctagaaaggg
ccgggctccctttgctgacccagagtcaacccagc
gattcctcccggggaggcggggggccccggccccg
gcccagagcgcatgcctatgccgggagctgggcga
ggacagggcacagccacagctgggtgcccaacggg
aaaggcggccg
Complementary sequence to lncGRS-2
(AC00 5624.2)
(SEQ ID NO: 39)
cccagccaaaaaaatttttaataattagccacatg
tgctggcacacacctgtagtcccagctcctcggga
ggctgaggcaggagaatggcttgcatccaggagtt
caagtctgcagtcagccatgattgcgccactgcac
tccagtgtgggcaacggagcgagaccccccatacc
cagcctttgcccgtgctgttccatccgccaggagc
gccgtttcctgcctgactgcgcggccctgggcctc
agtcttgcgctctgcaaaccggggtctcgaatcta
ccccagccctcctgtctcggcgctggggaggggct
tcgggtgcccagagccatccccgcgggacccccag
ggactggactccaagtcccag
Complementary sequence to lncGRS-3
(PDXDC2P)
(SEQ ID NO: 40)
ttttttttttttttttttttttttttttttttttt
ttttttttttttttttttttttttttttttttttt
ttttttttttttttttttttttttttttttttttt
tttttttttttttatttttatattatttatttatt
ctttgttagtttgtttttggagtgtgggttttgtt
ttgttttttgatttttttcttttgttccactcaca
gatttcttcttattgcccaggcttgagtgcaatga
cctgttctcagcttactcaacctctgcctcttggg
tttcagtgatttttctgcctcggcctcctgagtag
ctaagggaggagtcttgagattatcatccacggag
ggtggaagaggagagggtggaaggggaataagaag
acattcgggaggtgtcttgaggctcagggagttat
cagttatagaatgttgttgagttggaggaggtggc
tggcggcccatcctgttttttaaagtttcagctgt
gaggtagggccagtagggcaatcctgaagaatgac
gatgctccgctgccgccattctgaccttctgttgc
tggtgatagatttttgcacctttccatcctccagg
tttcaaaatagcagtatcagtgtcataatatcacc
cttccactgagtactgccgacagctggggagtaaa
gaaaagtcattgggacacactgttgtctccacatg
ccactgtgtctgtctgcaaatgtaggcaggctggg
gtcctgccccagggaagacagagtcataacagagt
aataaagaggcatgtttgagacacaggagtgtcta
tgtctatcctcattcctccctcacagccatcacca
gagcatgtttcttgcaccaggtcaacagacagtaa
gagaggcatgaaaagcccattgtccacacatgttg
cagcttctttttggagaatgttttccaggcctttt
atgttctgtctctgattctcggaactctgcaaggt
cagtgtgaccaccctgctccaaatctaagaaaaca
gaggtttccagaggaaggagaaattgtgcccgggg
tcacacagcttgcaagaggcagagtggaagttgat
tccagctctgcctgcaggaccctctcatttcccct
gtttcccttcttgataaaggatcttcttcactctg
gaggtgccacccatgagaacaaagagctctgcaga
gatgtggattcctgaagagctgcaggggaactggg
agagggttttctgacagaacaatcttacctcaaga
agtcagttaggcatggctgtaatatttcttttcac
tcccaggtaataccaaattgtaagtgcactaggac
ataaagaatacttttgtccatggaaaaatgaggtg
ggaattctaaacaaagcaagttttaaaactgtttc
acttcaagtgtacaagtcccatcacgtgtaatcat
aggactcggcagcttttgaaggtacagaggccaca
caagaaccagcttagctgagcatcatttaaggcct
tcatttggaattgtccctgtgggtaataagttaca
ttcactcttcactaatttacagtcagggcccattt
gctattacaaatatggaacctctgacactttgaat
attagatcaggggccccactgggtggggatgaagg
tgtttttgcacaacacggttaccaacagggatggg
actgtgatgcttgtaggcagccttcctctctgcca
tctccctctgcagggcttgagcacagagccagttg
caaagaggctttcttgcagagggggtgattgttct
ttggcggtcttcctctttccattgattttgtaatg
atggttgactttctttgtgaccttcatcttacgga
ttttagcttgaacttttgtttccgcctgtttccat
aaagtacagatgtcttccaggccaattttaattcc
ttcctgtctacggcggttggacctcctagctctct
gctgtacatccctggatctatcatgtccatttcga
gaccagaagatagtcttcaggaaaggcacacaaag
gtaaattgtgcagaagagaatggtaaatttgtaac
gtcccagaaacacaataatgataattatccaagga
cttccaccaaagtcagtctcacgatgacgatggtc
agccagagtattgataacaggggtgggctgatggc
tgataaatcccagcaggagccagaagaggagccaa
aagcgcagccaccgcacccgcatgtcctggtcctt
tcagggcgccctgaggcagccaggacagaggtgga
ggtggcttagggcaggggggagggaaggggatggg
accggggccagatctgagttggggagggggagggg
aaggggaggggaaggggggaagtaagggaagggaa
aggaggagaagggggctgttgggcacctgatttct
ggtcctccccaaccagctccctgtccctgcttctg
ggcgctccttccttcctgagctcccagggttcctc
aaggtcaccttcctcaggtactcgcatgggcatgt
cactgactttacgtgctgctgcagctccttggtga
gcgggccctggtcatgggacaggaactgtggggtc
aggacgatagagagcttcaccatttgcagaatgag
aacaggggctcatcatgagtgccaacctcagtttt
cttctgtgtgaaacaggaacaatagtttcattcct
cattcagtttctctcaaggtttcacgagatcatac
ctgctgcggtcatcaaagggctgaaccgcaaacac
gtgccctcagcttccagatccatgactgtgaggcc
gctcgcaggcaccagctgcttcagctgttctccca
gccagagattcagcgcatcacacgagtgcctctcc
cggccgactgctgaaggtgtcatgttgggcactgg
gacggctttaaacactggatctgagcctggtaatt
cttggaaaaatctgaacaccaccactggggagctg
agctcatcttccaccaagattttgatgtaattcac
tttcttcaaactttcctgcaaccattgactcagtt
gacaggcatgcttgatcctctccacaaacccatca
agtcccaagtattgtaaagataaccacagaggcag
ggcacggagtttgtctgtgggcttatttgatataa
gaccagcaactaaagtcaaggcagggtcgtgttta
tacagtgtcaccgcaggaacagctggcaaacccag
ccacgggccaggagtcatcgtcatgctatcacatt
tggctgcagccagcactgatgaggagacataaccc
agagccaatgttgccagattcacaccctccacatg
aagccatatgccatactgctcacagagttctttca
atctcccaatcttgtctgtgtgtcctactgctgcc
gttcctgcatttgcgacaagcaacaggggcagtct
tcctcgctctatatcatctttaatcagtttctcca
ggaaggcaacatccatctgatgctgggatccaaac
atagtgttacagggtacacggcacaagcaggggaa
gggcaagccgagctgattacaaaggtattggccca
ggccaggtctagcagcagcactaagatatatgaca
ggcttcttgttatataacccactgaagccatccac
tacgaagtcttcatattgagaatgaatggcaagcc
tacatatctttgcaagtccttctctttcctcttcg
tggaaataagcacacccattttcatatctgaaaat
tctgcatagccataaggtggtatctgaaagtatcc
tagttgtaagttttctcagcttctcttcgtccaga
gtcaaaatataagctcccagactatgtcccaacac
agccacatgaccttgttctccaatattttggattc
tggggctctggggctcctcatcttcatctccatgc
atcagattctgaactaactggaggatgctcaccat
atcttgtccactgccctggagtgggcctggaatat
ctctggatatgagcttctttccattttcctcttct
gttcttctctgactgtcctccagcatcttcactgc
ctccttcaagttttttcccatttcagctaacgtgg
ggtctgctatcttctccagggacgcgtccatggct
gaggcggccggtggtggcccggcggtcccgggctc
tacttcctcctcccgcgccccgcagctctttccac
gctggcagcgctgacgtcccccgcgcccgcggcca
gcggggcggagaagaggaggagctgccaggcggcg
ccgccggctgccgaacctgatggttgag
Complementary sequence to lncGRS-4
(RP11-195F19.9)
(SEQ ID NO: 41)
taggctaggtaatgctgtaataacaaatgacatcc
aaatctgaggagtgtaatacaacaaaattctattc
cccatgcataccaagcctgctgtgggtccaggcaa
gtctccagcacagttgtagtccatgtggctgctca
agttttcaagtgttttgatctgttggcagcctctt
catctcaacacaggcttccatggtcgctccagtca
aggaagacagcaagggaaattcttgcttaaatgtt
tttgtccacaagtgacatatgccatttctgttcac
atttcattggccaaagtgggtcacacagccatacc
taacgtcttgatggtaactcccaagatgagaagta
aaaccagaatcttgctgagtagtagtaataccacc
acaacaacacgatcacaaggtcccacaataggccg
tctgcaggctgaggagcaaggagagacagtccgag
ttccaaaactgaagaacttggagtccaacattcaa
gggcaggaaacatccaggatgggagaaagatgtag
gctgggaggctaggccagtctctcttttcacattt
ttctgagaactgggcagcctcgtcctgagaatgac
ggagaaggggacctggggctagggaatccctgatt
caatcatgccgctccccaggagacgttcggagccc
Complementary sequence to lncGRS-5
(SNHG1)
(SEQ ID NO: 42)
tatgtaatcaatcattttattattttcatcaacaa
ccaacacagcaacacaaattgacagccagtcctca
agggaaccttttttgaagtgaagagcaaggccctg
aatgagctacctacatggaacatagtatttgcaat
atgcaaatggagacctaaaggctcatgacgggaac
agaaggtggctgtatacagaggaacagacacgaag
tggagttatgggaagttcaatcaacaattggcaac
aagctaggtcagtagtttacgaaagtggtcatcta
tacagtgcctgagtttgggttctgggcctggatca
tgtaagaaaggcagcaaagtctgggtgccatgaat
cacacctgcaatcccagcactttggaaatctgtgg
tgagaggatctcttaaagctatggccttgaaacca
gcctgggtaacaaaaccaagcccttgtatctaaaa
aacaaaagggcaggtagattccagaaaataatgtt
gcaggaagggggtgataaaatacagaaatgtgata
cacagcaaaccctcaactgctgtttcattggctcc
cagtgtcttaatctgtgtctgctaacactttaagg
tacatctgaaataccccccaaacccagaaagcttt
tcaacagctaggttgtccaagaacttggaaaattc
accttctgatgtcctccaagacagattccattttt
tatacaccttatttgctcagacctgtaacttcagc
ctggagtgaacacagacacctagttttcctcaaac
tcctcttgggctttagagagaaggtgctggccctt
tgagccaagcaggttattggttagtagtacctctc
agctctgaaggcttttcaatggtatatagcaacaa
gctgctctgtaagatagaggcagactgtcatcagg
aatacctgtattcaccctgggaggcagctgaattc
cccaggatagattatgagatgctggaacattactg
taacgctggctttgcataaagatgggtcttgtccc
attccttcaccagtaagctcttgtgggctgaacat
tgcaacaacagagctctcttcggttccaacgtgcg
cgtaaatcctgcagacaggagcccaataagacatt
aggtcaactcagggcatttcgaaaacgctattttc
tgaacagacggctaagcaagaggcttgcagggccc
atctcctcagagttatttatcctcacggagctcag
tacaggtctgtgaaaaggtcctcatcataggaagg
gggacctacacggcccgccatcgccattttgtcct
tcacaaagccccactgctacttttaagcctcgagg
ccacgcacaagaccttatccgccagtcgccctggc
caggcag
Complementary sequence to lncGRS-6
(RP1-122P22.2)
(SEQ ID NO: 43)
aagcgttgcttccatctgacaatttcctggttggg
cagctctgtaggttgctggtggtcactgctatcat
tcagatcatccgcaattttcccctgatcttctgcc
ccagcccttcacgccagcatctcttctcctctccc
actgcagttcagcccaacctccccagcgttctgaa
gggcttgcacattttatcttttccggtagatgttc
agaagtccagatctctcgtctccatctctcgcggg
ccctccccgcaggtcccggaggcgggggcgaccgg
tggccccaaagacgagcgcggggctgggtttcccc
gcccgggaggagcaagaggaggaggaggaagagga
ggaagggggagaaaggaggggaggggaggggagag
gagaggaaagaggaggaggggagggaaggg
Complementary sequence to lncGRS-7
(MIR210HG)
(SEQ ID NO: 44)
caatgtttaagaaaatttatagatttgtgttgggc
tgcattcaaagctgttccatgggccaacagttgga
caagtttgccctagatcatggggtctttgtgctct
gagaatgggcgagtttgggatggactcagctttct
gttggttggccctggatctggatctggtgctgccc
cctttctctggagcacacaaagggaacaagaaggc
ctggacttgggcaaggaagccatccacagggctcc
ccagaagccagggccaccaggagctcactgctgtg
cagaggcaactcggcttggttatttcttgcctgag
gcagggaacagggaatgcagggaagccaggcagca
gcaggaggctccaaacgctggtgtcagcaagccag
aacccccacctgacttcactcctgtctgcccagct
cacagtcgcctgtcactgggaggtccttggggcct
ggacctgcctgacctgtggccccagaaggggctaa
gcccagagtcacatgggatggagcctggaatgcat
aggccaagtgggttgtgcctgggcagcaggcctgg
gatatgaggggcctactcctccatccacagccccg
acccacattctggagggatggtcgtacctctgtga
ccagccctccacaccgtcacataggccagcagggg
gcaatgccaggtgccatccagagcccacccccatg
tcctcccggaggtcatgagctctgccctacgtacc
ccagccccacaatgggaaggaggcatggagctggc
agagcatgcgccacgccgtccctgtgtgtgtttct
gaggaactcctgttcactctgaggccactgcctag
gtgcccgtccctggtctgctgctgctggctctgat
gaagctaaccagccttcttccccaccaagagggtg
tcccagcagcagctatggctatgttccggcggggg
gaaggcggggagccttcctcctcactggctgtttc
atttttttatttttttgaggcagagtctctcgtca
cccaggctggagtgcagcggcgtaatctcgtctcg
ctgcaacctccacctcccaggttcaagcgattctc
ttgcctcagccttccaagtagctgggattacaggt
gcccaccaccatgcctagctaatttttgtattttt
agtagagacgaggtttcaccatgttgcccaggctg
gtcttgaactcctgatctcaagtgagcgatcaccc
gcgtctgcctcctgaagtgttgggattacgggtgt
gagccaccacacccggcctctcactggctgtttca
ggtgaaccaaatcccatggccccaaaaagtggcca
gagtgcttcagatcactcagcaaacctttgagttg
ctgaaaagtttacttttgagttttaaactgtactt
tgaaaactatattgtgaactgtgttgacatgagga
aaggctgggctccctgaaaacatccagttccacag
cagggcgggcccaagcccagccaatccccaggcaa
tgcagggcagggatcccacctggtgtaagtacggg
cagagggcagagccaggctgtatcgaggggcggcg
ctgggaccctcctcgcccagagttcctactcatcc
ccagcacagaagtgctctgtagggccagctgagga
caccccggttcactgagggtggcccacagtaagtc
gccgtctggcagtaagttagctctgcaggtgtgga
ccccaagaccacacacagcctttctcaggtgcagg
tcctgccaagcctcacttcgcagtggtgacaggca
accccgtcagggcagggagcttccaaaggccagtc
cgagtccatgccaggcgtccactaaatctgcctct
ccctctccagcgagcaggtgcagccctcaactcct
gcaggcctgcgaccccggcgcggggccgtcgccgg
ctctcggcccaacttcagctgcggccgctggcacc
ctctc
Complementary sequence to lncGRS-8
(SNHG12)
(SEQ ID NO: 45)
ttttttttttttttttttttacttttattagtctt
tttattcttcttttattgatcacctctgaagttca
gtagcacactgcataattttttgcattagcatgct
gttgtttctacctaaaatgaccggggtccttgcct
tctgcttcccatagagattgtcccaactccatagg
tccatagtcacaaatgaggacagctttacatgtaa
catgaatcttaaagcacagctccagaaacaagctc
acctcctcagtatcacacactgccaggaaacttgg
gggcaaccaggtcccctgcatttcatcactgtcat
taccccggaagtcctcgatcaccagaaaaatcacc
ttctgtcatcttaagagtcttcatctgcttaagta
cgccgggatctctgtagactaagtcagtcatcctg
taggccccactgcccacgctgtgccaatagctggt
gtgctttttctcagtcttgatgggaccgttttatc
cgcctgtcagcccagagcgcagcgttgcgcagtcc
gaagcgagagaagggcgcggcccccgcgggctccc
gcccctctcactgccgagggggcctttcggagctc
gggcccaccgggttggaaggcccggctcctccgtg
ccacattcaccaccatctcgaggccgtgggaggaa
aaacccggcgagtgcgcgccagcagcagcgactag
cggggaacggcgcgcaggaaggcaggcagaggctt
gcagctagtaagtcgacaccggg
Complementary sequence to lncGRS-9
(RP11-339B21.10)
(SEQ ID NO: 46)
tacacgtgcacatatatttggctatagcagagaac
acaaacaaggaatccatgtcccatctcctggaagg
gctgcccttggagggcccagttcccattctctcct
gccgtgtccagcaacctctttcctgcgctcacact
cccagcccctgggccgaggcttcttggccaagtac
tgagcacatgctaagccccaggcttccttctttct
gctcctaaacaccttcctgcctcagggccttggca
cccactgttctcttctgcctagaagcctcttctcc
cggcccctcaatggtgaggtccattctgaccccct
cccacacgaggcacctctcaaagtggcccatacct
ctcccctccactgcaagcttggttttgatttccat
aatcccgctctggtccctgagccc

In some embodiments, the nucleic acids that are complementary or at least substantially complementary, to any one of the lncGRSs disclosed herein comprise at least about 70%, 75%, 80%, 85%, 90%, 95%, 96%, 97%, 98%, 99% or 100% sequence identity to SEQ ID NO: 38, or a functional fragment thereof In some embodiments, the nucleic acids that are complementary or at least substantially complementary, to any one of the lncGRSs disclosed herein comprise at least about 70%, 75%, 80%, 85%, 90%, 95%, 96%, 97%, 98%, 99% or 100% sequence identity to SEQ ID NO: 39, or a functional fragment thereof In some embodiments, the nucleic acids that are complementary or at least substantially complementary, to any one of the lncGRSs disclosed herein comprise at least about 70%, 75%, 80%, 85%, 90%, 95%, 96%, 97%, 98%, 99% or 100% sequence identity to SEQ ID NO: 40, or a functional fragment thereof. In some embodiments, the nucleic acids that are complementary or at least substantially complementary, to any one of the lncGRSs disclosed herein comprise at least about 70%, 75%, 80%, 85%, 90%, 95%, 96%, 97%, 98%, 99% or 100% sequence identity to SEQ ID NO: 41, or a functional fragment thereof. In some embodiments, the nucleic acids that are complementary or at least substantially complementary, to any one of the lncGRSs disclosed herein comprise at least about 70%, 75%, 80%, 85%, 90%, 95%, 96%, 97%, 98%, 99% or 100% sequence identity to SEQ ID NO: 42, or a functional fragment thereof. In some embodiments, the nucleic acids that are complementary or at least substantially complementary, to any one of the lncGRSs disclosed herein comprise at least about 70%, 75%, 80%, 85%, 90%, 95%, 96%, 97%, 98%, 99% or 100% sequence identity to SEQ ID NO: 43, or a functional fragment thereof. In some embodiments, the nucleic acids that are complementary or at least substantially complementary, to any one of the lncGRSs disclosed herein comprise at least about 70%, 75%, 80%, 85%, 90%, 95%, 96%, 97%, 98%, 99% or 100% sequence identity to SEQ ID NO: 44, or a functional fragment thereof. In some embodiments, the nucleic acids that are complementary or at least substantially complementary, to any one of the lncGRSs disclosed herein comprise at least about 70%, 75%, 80%, 85%, 90%, 95%, 96%, 97%, 98%, 99% or 100% sequence identity to SEQ ID NO: 45, or a functional fragment thereof In some embodiments, the nucleic acids that are complementary or at least substantially complementary, to any one of the lncGRSs disclosed herein comprise at least about 70%, 75%, 80%, 85%, 90%, 95%, 96%, 97%, 98%, 99% or 100% sequence identity to SEQ ID NO: 46, or a functional fragment thereof.

In some embodiments, the method further comprises obtaining a sample from the subject. Any type of samples suitable for detecting the presence or absence of a nucleic acid, particularly any one of the lncGRSs disclosed herein, may be used. In some embodiment, suitable samples include, but not limited to, a human tissue sample, serum, plasma, blood draw, brushing, biopsy, or surgical resection.

In some embodiments, the method further comprises a step of correlating the binding between the lncRNA in the sample and the nucleic acid complementary to the lncRNA to the subject having or being diagnosed with malignant glioma. In some embodiments, prior to this correlating step, the method further comprises normalizing the binding between the lncRNA in the sample and the nucleic acid complementary to the lncRNA against the binding between the lncRNA in a sample of a control subject. In some embodiments, prior to this correlating step, the method further comprises normalizing the intensity or absorbance of a signal obtained from measuring the binding between the lncRNA in the sample and the nucleic acid complementary to the lncRNA against the intensity or absorbance of a signal obtained by measuring the binding between the lncRNA or non-lncRNA in a sample of a control subject. In some embodiments, wherein normalizing is performed In some embodiments, the control subject is a healthy subject. In some embodiments, the control subject is a subject known to have malignant glioma.

In some embodiments, the method further comprises a step of providing the subject a treatment regime to manage the malignant glioma. Treatment regimes may include, but not limited to radiotherapy, chemotherapy, surgery, and targeted therapy, or any combination thereof. In some embodiments, the treatment regime includes at least radiotherapy. In other embodiments, the treatment regime includes administering a therapeutically effective amount of any of the pharmaceutical compositions disclosed herein. In some embodiments, the treatment regime includes a combination of at least radiotherapy and administering a therapeutically effective amount of any of the pharmaceutical compositions disclosed herein.

In another aspect, the present disclosure provides a method of treating malignant glioma in a subject in need thereof comprising administering to the subject a therapeutically effective amount of an agent that knockdowns expression of any one of the lncGRSs disclosed herein in the subject. In another aspect, the present disclosure provides a method of treating malignant glioma in a subject in need thereof comprising administering to the subject a therapeutically effective amount of the agent and a dose of radiation. In some embodiments, the dose of radiation used for such treatments is the same as routinely used for treatment of malignant glioma as determined by a physician. In some embodiments, the dose of radiation used for such treatments is less than what routinely used for treatment of malignant glioma as determined by the physician. The expression of any one of the lncGRSs disclosed herein may be knocked down, or depleted, by any method known in the art. In some embodiments, the expression of any one of the lncGRSs disclosed herein in the subject is knocked down by an interference mechanism using, for example, a microRNA (miRNA) or a small interfering RNA (siRNA). In some embodiments, the expression of any one of the lncGRSs disclosed herein in the subject is knocked down by an oligomer, such as an antisense oligonucleotide (ASO) or a morpholino. Other molecules capable of knocking down gene expression may also be used and thus are also encompassed herein. In some embodiments, the target lncGRS being knocked down comprises a nucleic acid sequence having at least about 70%, 75%, 80%, 85%, 90%, 95%, 96%, 97%, 98%, 99% or 100% sequence identity to SEQ ID NO: 1. In some embodiments, the target lncGRS being knocked down comprises a nucleic acid sequence having at least about 70%, 75%, 80%, 85%, 90%, 95%, 96%, 97%, 98%, 99% or 100% sequence identity to SEQ ID NO: 2. In some embodiments, the target lncGRS being knocked down comprises a nucleic acid sequence having at least about 70%, 75%, 80%, 85%, 90%, 95%, 96%, 97%, 98%, 99% or 100% sequence identity to SEQ ID NO: 3. In some embodiments, the target lncGRS being knocked down comprises a nucleic acid sequence having at least about 70%, 75%, 80%, 85%, 90%, 95%, 96%, 97%, 98%, 99% or 100% sequence identity to SEQ ID NO: 4. In some embodiments, the target lncGRS being knocked down comprises a nucleic acid sequence having at least about 70%, 75%, 80%, 85%, 90%, 95%, 96%, 97%, 98%, 99% or 100% sequence identity to SEQ ID NO: 5. In some embodiments, the target lncGRS being knocked down comprises a nucleic acid sequence having at least about 70%, 75%, 80%, 85%, 90%, 95%, 96%, 97%, 98%, 99% or 100% sequence identity to SEQ ID NO: 6. In some embodiments, the target lncGRS being knocked down comprises a nucleic acid sequence having at least about 70%, 75%, 80%, 85%, 90%, 95%, 96%, 97%, 98%, 99% or 100% sequence identity to SEQ ID NO: 7. In some embodiments, the target lncGRS being knocked down comprises a nucleic acid sequence having at least about 70%, 75%, 80%, 85%, 90%, 95%, 96%, 97%, 98%, 99% or 100% sequence identity to SEQ ID NO: 8. In some embodiments, the target lncGRS being knocked down comprises a nucleic acid sequence having at least about 70%, 75%, 80%, 85%, 90%, 95%, 96%, 97%, 98%, 99% or 100% sequence identity to SEQ ID NO: 9.

In some embodiments, the expression of any one of the lncGRSs disclosed herein in the subject is knocked down by a nucleic acid that is at least about 70%, 75%, 80%, 85%, 90%, 95%, 96%, 97%, 98%, 99% or 100% sequence identity to SEQ ID NO: 38, or a functional fragment thereof. In some embodiments, the expression of any one of the lncGRSs disclosed herein in the subject is knocked down by a nucleic acid that is at least about 70%, 75%, 80%, 85%, 90%, 95%, 96%, 97%, 98%, 99% or 100% sequence identity to SEQ ID NO: 39, or a functional fragment thereof In some embodiments, the expression of any one of the lncGRSs disclosed herein in the subject is knocked down by a nucleic acid that is at least about 70%, 75%, 80%, 85%, 90%, 95%, 96%, 97%, 98%, 99% or 100% sequence identity to SEQ ID NO: 40, or a functional fragment thereof In some embodiments, the expression of any one of the lncGRSs disclosed herein in the subject is knocked down by a nucleic acid that is at least about 70%, 75%, 80%, 85%, 90%, 95%, 96%, 97%, 98%, 99% or 100% sequence identity to SEQ ID NO: 41, or a functional fragment thereof In some embodiments, the expression of any one of the lncGRSs disclosed herein in the subject is knocked down by a nucleic acid that is at least about 70%, 75%, 80%, 85%, 90%, 95%, 96%, 97%, 98%, 99% or 100% sequence identity to SEQ ID NO: 42, or a functional fragment thereof In some embodiments, the expression of any one of the lncGRSs disclosed herein in the subject is knocked down by a nucleic acid that is at least about 70%, 75%, 80%, 85%, 90%, 95%, 96%, 97%, 98%, 99% or 100% sequence identity to SEQ ID NO: 43, or a functional fragment thereof In some embodiments, the expression of any one of the lncGRSs disclosed herein in the subject is knocked down by a nucleic acid that is at least about 70%, 75%, 80%, 85%, 90%, 95%, 96%, 97%, 98%, 99% or 100% sequence identity to SEQ ID NO: 44, or a functional fragment thereof In some embodiments, the expression of any one of the lncGRSs disclosed herein in the subject is knocked down by a nucleic acid that is at least about 70%, 75%, 80%, 85%, 90%, 95%, 96%, 97%, 98%, 99% or 100% sequence identity to SEQ ID NO: 45, or a functional fragment thereof In some embodiments, the expression of any one of the lncGRSs disclosed herein in the subject is knocked down by a nucleic acid that is at least about 70%, 75%, 80%, 85%, 90%, 95%, 96%, 97%, 98%, 99% or 100% sequence identity to SEQ ID NO: 46, or a functional fragment thereof.

In some embodiments, the expression of any one of the lncGRSs disclosed herein in the subject is knocked down by a nucleic acid that is a fragment of any of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 and SEQ ID NO: 46. In some embodiments, such fragments of any of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 and SEQ ID NO: 46 comprise at least about 10, 11, 12, 13, 14, 15, 16, 17, 18, 19, 20 or more nucleotides. In some embodiments, such fragments of any of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 and SEQ ID NO: 46 comprise more than about 20, 30, 40, 50, 60, 70, 80, 90 or 100 nucleotides. In some embodiments, such fragments of any of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 and SEQ ID NO: 46 comprise from about 10 to about 200 nucleotides. In some embodiments, such fragments of any of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 and SEQ ID NO: 46 comprise from about 10 to about 150 nucleotides. In some embodiments, such fragments of any of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 and SEQ ID NO: 46 comprise from about 10 to about 100 nucleotides.

In some particular embodiments, the target lncGRS being knocked down comprises a nucleic acid sequence having at least about 70%, 75%, 80%, 85%, 90%, 95%, 96%, 97%, 98%, or 99% sequence identity to SEQ ID NO: 1. In other embodiments, the target lncGRS being knocked down comprises the nucleic acid sequence of SEQ ID NO: 1. In some embodiments, the agent that knockdowns expression of the lncGRS is an ASO comprising a nucleic acid sequence having at least about 70%, 75%, 80%, 85%, 90%, 95%, 96%, 97%, 98%, or 99% sequence identity to SEQ ID NO: 10 or SEQ ID NO: 11. In some embodiments, the agent that knockdowns expression of the lncGRS is an ASO comprising the nucleic acid sequence of SEQ ID NO: 10 or SEQ ID NO: 11.

In some embodiments, the subject being treated is concurrently under radiation therapy. Due to the nature of being a radiotherapy sensitizer, administering the agent that knockdowns expression of any one of the lncGRSs disclosed herein would synergize the effect of the radiation therapy the subject is receiving. In some embodiments therefore, the administration of the agent that knockdowns expression of any one of the lncGRSs disclosed herein increases the effect of the radiation therapy compared to administering the radiation therapy alone. In some embodiments, the malignant glioma being treated is glioblastoma or diffuse intrinsic pontine gliomas.

In yet another aspect, the present disclosure provides a method of inhibiting growth and/or proliferation of glioma cells in a subject in need thereof comprising administering to the subject a therapeutically effective amount of radiotherapy or any of the agents described above to knock down the expression of any one of the lncGRSs disclosed herein in the subject; or radiotherapy with any of the agents described above to knock down the expression of any one of the lncGRSs disclosed herein. In some embodiments, the target lncGRS being knocked down comprises a nucleic acid sequence having at least about 70%, 75%, 80%, 85%, 90%, 95%, 96%, 97%, 98%, 99% or 100% sequence identity to SEQ ID NO: 1. In some embodiments, the target lncGRS being knocked down comprises a nucleic acid sequence having at least about 70%, 75%, 80%, 85%, 90%, 95%, 96%, 97%, 98%, 99% or 100% sequence identity to SEQ ID NO: 2. In some embodiments, the target lncGRS being knocked down comprises a nucleic acid sequence having at least about 70%, 75%, 80%, 85%, 90%, 95%, 96%, 97%, 98%, 99% or 100% sequence identity to SEQ ID NO: 3. In some embodiments, the target lncGRS being knocked down comprises a nucleic acid sequence having at least about 70%, 75%, 80%, 85%, 90%, 95%, 96%, 97%, 98%, 99% or 100% sequence identity to SEQ ID NO: 4. In some embodiments, the target lncGRS being knocked down comprises a nucleic acid sequence having at least about 70%, 75%, 80%, 85%, 90%, 95%, 96%, 97%, 98%, 99% or 100% sequence identity to SEQ ID NO: 5. In some embodiments, the target lncGRS being knocked down comprises a nucleic acid sequence having at least about 70%, 75%, 80%, 85%, 90%, 95%, 96%, 97%, 98%, 99% or 100% sequence identity to SEQ ID NO: 6. In some embodiments, the target lncGRS being knocked down comprises a nucleic acid sequence having at least about 70%, 75%, 80%, 85%, 90%, 95%, 96%, 97%, 98%, 99% or 100% sequence identity to SEQ ID NO: 7. In some embodiments, the target lncGRS being knocked down comprises a nucleic acid sequence having at least about 70%, 75%, 80%, 85%, 90%, 95%, 96%, 97%, 98%, 99% or 100% sequence identity to SEQ ID NO: 8. In some embodiments, the target lncGRS being knocked down comprises a nucleic acid sequence having at least about 70%, 75%, 80%, 85%, 90%, 95%, 96%, 97%, 98%, 99% or 100% sequence identity to SEQ ID NO: 9.

In some particular embodiments, the target lncGRS being knocked down comprises a nucleic acid sequence having at least about 70%, 75%, 80%, 85%, 90%, 95%, 96%, 97%, 98%, or 99% sequence identity to SEQ ID NO: 1. In other embodiments, the target lncGRS being knocked down comprises the nucleic acid sequence of SEQ ID NO: 1. In some embodiments, the agent that knockdowns expression of the lncGRS is an ASO comprising a nucleic acid sequence having at least about 70%, 75%, 80%, 85%, 90%, 95%, 96%, 97%, 98%, or 99% sequence identity to SEQ ID NO: 10 or SEQ ID NO: 11. In some embodiments, the agent that knockdowns expression of the lncGRS is an ASO comprising the nucleic acid sequence of SEQ ID NO: 10 or SEQ ID NO: 11.

In some embodiments, the subject in need thereof has or is suspected to have malignant glioma. In some embodiments, the malignant glioma is glioblastoma or diffuse intrinsic pontine gliomas. In some embodiments, the subject having or being suspected to have malignant glioma is concurrently under radiation therapy. Again, because of the nature of being a radiotherapy sensitizer, the agent that knockdowns expression of any one of the lncGRSs disclosed herein would synergize the effect of the radiation therapy the subject is receiving. In some embodiments therefore, the administration of the agent that knockdowns expression of any one of the lncGRSs disclosed herein increases the effect of the radiation therapy compared to administering the radiation therapy alone.

In some embodiments, the agent that knockdowns expression of any one of the lncGRSs disclosed herein comprises at least about 70%, 75%, 80%, 85%, 90%, 95%, 96%, 97%, 98%, 99% or 100% sequence identity to SEQ ID NO: 38. In some embodiments, the agent that knockdowns expression of any one of the lncGRSs disclosed herein comprises at least about 70%, 75%, 80%, 85%, 90%, 95%, 96%, 97%, 98%, 99% or 100% sequence identity to SEQ ID NO: 39. In some embodiments, the agent that knockdowns expression of any one of the lncGRSs disclosed herein comprises at least about 70%, 75%, 80%, 85%, 90%, 95%, 96%, 97%, 98%, 99% or 100% sequence identity to SEQ ID NO: 40. In some embodiments, the agent that knockdowns expression of any one of the lncGRSs disclosed herein comprises at least about 70%, 75%, 80%, 85%, 90%, 95%, 96%, 97%, 98%, 99% or 100% sequence identity to SEQ ID NO: 41. In some embodiments, the agent that knockdowns expression of any one of the lncGRSs disclosed herein comprises at least about 70%, 75%, 80%, 85%, 90%, 95%, 96%, 97%, 98%, 99% or 100% sequence identity to SEQ ID NO: 42. In some embodiments, the agent that knockdowns expression of any one of the lncGRSs disclosed herein comprises at least about 70%, 75%, 80%, 85%, 90%, 95%, 96%, 97%, 98%, 99% or 100% sequence identity to SEQ ID NO: 43. In some embodiments, the agent that knockdowns expression of any one of the lncGRSs disclosed herein comprises at least about 70%, 75%, 80%, 85%, 90%, 95%, 96%, 97%, 98%, 99% or 100% sequence identity to SEQ ID NO: 44. In some embodiments, the agent that knockdowns expression of any one of the lncGRSs disclosed herein comprises at least about 70%, 75%, 80%, 85%, 90%, 95%, 96%, 97%, 98%, 99% or 100% sequence identity to SEQ ID NO: 45. In some embodiments, the agent that knockdowns expression of any one of the lncGRSs disclosed herein comprises at least about 70%, 75%, 80%, 85%, 90%, 95%, 96%, 97%, 98%, 99% or 100% sequence identity to SEQ ID NO: 46.

In some embodiments, the agent that knockdowns expression of any one of the lncGRSs disclosed herein a fragment of any of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 and SEQ ID NO: 46. In some embodiments, such fragments of any of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 and SEQ ID NO: 46 comprise at least about 10, 11, 12, 13, 14, 15, 16, 17, 18, 19, 20 or more nucleotides. In some embodiments, such fragments of any of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 and SEQ ID NO: 46 comprise more than about 20, 30, 40, 50, 60, 70, 80, 90 or 100 nucleotides. In some embodiments, such fragments of any of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 and SEQ ID NO: 46 comprise from about 10 to about 200 nucleotides. In some embodiments, such fragments of any of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 and SEQ ID NO: 46 comprise from about 10 to about 150 nucleotides. In some embodiments, such fragments of any of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 and SEQ ID NO: 46 comprise from about 10 to about 100 nucleotides.

In some embodiments, the agent that knockdowns expression of any one of the lncGRSs disclosed herein a miRNA, a siRNA or an ASO comprising at least about 70% sequence identity to SEQ ID NO: 38 or a functional fragement thereof. In some embodiments, the agent that knockdowns expression of any one of the lncGRSs disclosed herein a miRNA, a siRNA or an ASO comprising at least about 70% sequence identity to SEQ ID NO: 39 or a functional fragement thereof In some embodiments, the agent that knockdowns expression of any one of the lncGRSs disclosed herein a miRNA, a siRNA or an ASO comprising at least about 70% sequence identity to SEQ ID NO: 40 or a functional fragement thereof In some embodiments, the agent that knockdowns expression of any one of the lncGRSs disclosed herein a miRNA, a siRNA or an ASO comprising at least about 70% sequence identity to SEQ ID NO: 41 or a functional fragement thereof In some embodiments, the agent that knockdowns expression of any one of the lncGRSs disclosed herein a miRNA, a siRNA or an ASO comprising at least about 70% sequence identity to SEQ ID NO: 42 or a functional fragement thereof. In some embodiments, the agent that knockdowns expression of any one of the lncGRSs disclosed herein a miRNA, a siRNA or an ASO comprising at least about 70% sequence identity to SEQ ID NO: 43 or a functional fragement thereof In some embodiments, the agent that knockdowns expression of any one of the lncGRSs disclosed herein a miRNA, a siRNA or an ASO comprising at least about 70% sequence identity to SEQ ID NO: 44 or a functional fragement thereof. In some embodiments, the agent that knockdowns expression of any one of the lncGRSs disclosed herein a miRNA, a siRNA or an ASO comprising at least about 70% sequence identity to SEQ ID NO: 45 or a functional fragement thereof. In some embodiments, the agent that knockdowns expression of any one of the lncGRSs disclosed herein a miRNA, a siRNA or an ASO comprising at least about 70% sequence identity to SEQ ID NO: 46 or a functional fragement thereof In some embodiments, the agent that knockdowns expression of any one of the lncGRSs disclosed herein comprises one or a plurality of modified nucleotides.

Administration of an agent that knockdowns expression of any one of the lncGRSs disclosed herein, such as a miRNA, siRNA, ASO or a morpholino, can be carried out using the various mechanisms known in the art, including naked administration and administration in pharmaceutically acceptable carriers, such as, but not limited to, solvent, aqueous solvent, non-aqueous solvent, dispersion media, diluent, dispersion, suspension aid, surface active agent, isotonic agent, thickening or emulsifying agent, preservative, lipid, lipidoids liposome, lipid nanoparticle, core-shell nanoparticles, polymer, lipoplexe peptide, protein, cell, hyaluronidase, and mixtures thereof. For example, lipid carriers for antisense delivery are disclosed in U.S. Pat. Nos. 5,855,911 and 5,417,978, and lipid nanoparticles for delivery of polynucleotides is described in U.S. Pat. No. 9,872,900, all of which are incorporated herein by reference. The administration may also be in form of a nanoparticle formulation as bescribed in, for instance, WO 2014/152211 and US2017/0173128, both are incorporated herein by reference. In general, the agent can be administered by intradermal, intravenous, intraperitoneal, intramuscular, subcutaneous or oral routes, or direct local tumor injection.

The amount of agent administered is one effective to knockdown expression of the target lncRNA, such as any one of the lncGRSs disclosed herein, in glioma cells. It will be appreciated that this amount will vary both with the effectiveness of the agent employed, and with the nature of any carrier used. The determination of appropriate amounts for any given composition is within the skill in the art, through standard series of tests designed to assess appropriate therapeutic levels.

### Pharmaceutical Compositions

According to another aspect, the present disclosure provides a pharmaceutical composition for treating malignant glioma in a subject in need thereof comprising (i) a therapeutically effective amount of any of the agents that knockdowns expression of any of the lncGRSs disclosed herein as described elsewhere herein, and (ii) a pharmaceutically acceptable carrier. In some embodiments, the agent comprised in the disclosed composition is capable of knocking down the expression of a lncGRS having at least about 70%, 75%, 80%, 85%, 90%, 95%, 96%, 97%, 98%, or 99% sequence identity to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 and SEQ ID NO: 9. In some embodiments, the agent comprised in the disclosed composition is capable of knocking down the expression of a lncGRS having at least about 70%, 75%, 80%, 85%, 90%, 95%, 96%, 97%, 98%, or 99% sequence identity to SEQ ID NO: 1. In some embodiments, the agent comprised in the disclosed composition is capable of knocking down the expression of a lncGRS comprising the nucleic acid sequence of SEQ ID NO: 1.

In some embodiments, the agent that knockdowns expression of the disclosed lncGRS is a miRNA, siRNA, antisense oligonucleotide (ASO), or morpholino. In some embodiments, the agent is an ASO comprising a nucleic acid sequence having at least about 70%, 75%, 80%, 85%, 90%, 95%, 96%, 97%, 98%, or 99% sequence identity to SEQ ID NO: 10 or SEQ ID NO: 11 and is capable of knocking down expression of a lncGRS having at least about 70%, 75%, 80%, 85%, 90%, 95%, 96%, 97%, 98%, or 99% sequence identity to SEQ ID NO: 1. In some embodiments, the agent is an ASO comprising the nucleic acid sequence of SEQ ID NO: 10 or SEQ ID NO: 11.

The pharmaceutical compositions of the present disclosure can further include one or more compatible active ingredients which are aimed at providing the composition with another pharmaceutical effect in addition to that provided by the agent that knockdowns expression of the lncGRS. “Compatible” as used herein means that the active ingredients of such a composition are capable of being combined with each other in such a manner so that there is no interaction that would substantially reduce the efficacy of each active ingredient or the composition under ordinary use conditions. Such one or more compatible active ingredients may include, but not limited to, chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc., or immunosuppressive agents, e.g., cyclosporin A, tacrolimus, mycophenolate, rapamycin, corticosteroids, etc. In some embodiments, the agents are administered in the form of a salt, e.g., a pharmaceutically acceptable salt. “Pharmaceutically acceptable salts” means physiologically and pharmaceutically acceptable salts of compounds, such as oligomeric compounds, i.e., salts that retain the desired biological activity of the parent compound and do not impart undesired toxicological effects thereto. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

The choice of carrier in the pharmaceutical composition may be determined in part by the particular method used to administer the composition. Accordingly, there is a variety of suitable formulations. For example, the pharmaceutical composition can contain preservatives. Suitable preservatives may include, for example, methylparaben, propylparaben, sodium benzoate, and benzalkonium chloride. In some embodiments, a mixture of two or more preservatives is used. The preservative or mixtures thereof are typically present in an amount of about 0.0001% to about 2% by weight of the total composition.

In addition, buffering agents in some aspects are included in the composition. Suitable buffering agents include, for example, citric acid, sodium citrate, phosphoric acid, potassium phosphate, and various other acids and salts. In some embodiments, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins 21^{st} ed. (May 1, 2005) or Remington's Pharmaceutical Sciences, 18^{th} or 19^{th} ed. published by the Mack Publishing Company of Easton, Pa., both are incorporated herein by reference.

In some embodiments, the pharmaceutical composition comprises the disclosed composition in an amount that is effective to treat or prevent the disease or condition, such as a therapeutically effective or prophylactically effective amount. Thus, in some embodiments, the methods of administration include administration of the disclosed composition at effective amounts. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

Although the descriptions of pharmaceutical compositions provided herein are principally directed to pharmaceutical compositions, which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation.

Pharmaceutical compositions that are useful in the methods of the disclosure may be prepared/formulated, packaged, or sold in formulations suitable for oral, rectal, vaginal, parenteral, topical, pulmonary, intranasal, intra-lesional, buccal, ophthalmic, intravenous, intra-organ or another route of administration.

According to some embodiments, the pharmaceutical compositions of the present disclosure may be administered initially, and thereafter maintained by further administrations. For example, according to some embodiments, the pharmaceutical compositions of the described invention may be administered by one method of injection, and thereafter further administered by the same or by different method.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations may include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt. For parenteral application, suitable vehicles consist of solutions, e.g., oily or aqueous solutions, as well as suspensions, emulsions, or implants. Aqueous suspensions may contain substances, which increase the viscosity of the suspension and include, for example, sodium carboxymethyl cellulose, sorbitol and/or dextran.

### Three-Dimensional Human Brain Organoid of Malignant Glioma

Another aspect of the present disclosure relates to the generation and use of three-dimensional (3D) human brain organoids of malignant glioma. Organoids are miniature three dimensional (3D) representations of their in vivo counterpart organs, and organoid-based models of cancer are emerging as a useful platform for the evaluation of therapeutics [25]. Different organoid platforms have their own important utility in different pre-clinical research uses.

Human brain organoids have been previously generated from pluripotent stem cell (PSC) populations, mimicking early stages of fetal brain development [26-28]. Such embryonic brain organoids have been useful to study the genetic mutations that cause GBM [29] and can also serve as a 3D tissue substrate for the growth of glial tumors [30]. However, embryonic brain organoids do not closely represent the mature brain tissue of glioma patients, limiting their utility for assessing potential drug toxicity to normal adult brain cells.

A “mature” human brain organoids (MBOs) can offer certain characteristics that distinguish them from embryonic brain organoids. In contrast to embryonic brain organoids that mimic early stages of fetal brain development, MBOs are assembled from cell populations that are more mature and postmitotic [54,55]. Because embryonic brain organoids contain a large proportion of proliferative precursor cells, radiation treatment and/or other traditional chemotherapies may not be well-tolerated by such normal but immature cells. A systematic three-dimensional coculture containing pre-differentiated human pluripotent stem cell (hPSC)-derived astrocytes (hAstros) combined with neurons generated from hPSC-derived neural stem cells (hNeurons) or directly induced via Neurogenin 2 overexpression (iNeurons) has been previously described. See Krencik et al. (2017) Stem Cell Reports. 9(6):1745-1753.

In contrast to embryonic brain organoids and MBOs, GBM-derived tumor organoids, which comprise of only tumor cells, are useful for the study of drug efficacy in 3D tissues. In some embodiments therefore, the present disclosure provides a method for generation of a human brain organoid model of malignant glioma for such uses. The disclosed method comprises seeding cells derived from glioblastoma (GBM) or diffuse intrinsic pontine gliomas (DIPG) onto a surface of a three-dimensional human brain organoid having one or more aspects of a mature human brain and allowing the cells to grow and form a tumor within the human brain organoid.

A 3D human brain organoid having one or more aspects of a mature human brain, or a MBO, can be generated using any methods known in the art, such as that disclosed in Krencik et al. (2017) Stem Cell Reports. 9(6):1745-1753. Aspects of a mature human brain may include, but not limited to, the presence of mature human astrocytes from human iPSCs (iAstrocytes) and/or mature cortical neurons with NGN2 induction (i3Neurons). In some embodiments, the 3D human brain organoid comprises iAstrocytes and/or i3Neurons. In some embodiments, the 3D human brain organoid comprises solely iAstrocytes. In some embodiments, the 3D human brain organoid comprises solely i3Neurons. In some embodiments, the 3D human brain organoid comprises iAstrocytes and i3Neurons in a ratio of about 1:1.

To generate a human brain organoid model of malignant glioma, cells derived from malignant glioma are first seeded onto a surface of the 3D human brain organoid described above and then allowed to grow, propagate, and form a tumor within the human brain organoid. Cells may be derived from any type of malignant gliomas, including but not limited to glioblastoma (GBM) or diffuse intrinsic pontine gliomas (DIPG). In some embodiments, the cells are derived from GBM. In other embodiments, the cells are derived from DIPG. In some embodiments where the cells are derived from GBM, the cells are GBM SF10360 cells. In some embodiments where the cells are derived from DIPG, the cells are DIPG SF8628 cells.

Also provided herein are the human brain organoid model of malignant glioma obtained by any of the methods described above and their use in screening therapeutics for treating gliomas. In some embodiments, the present disclosure further provides a method of screening an agent capable of synergizing with radiation to reduce growth of glioma cells by using the human brain organoid model of malignant glioma disclosed herein. The method comprises adding a candidate agent to a human brain organoid model of malignant glioma disclosed herein, treating the human brain organoid model of malignant glioma with a clinically therapeutic level of radiation, and detecting the effectiveness of the candidate agent in synergizing with the radiation to reduce growth of glioma cells by comparing with a corresponding control treated with the radiation alone without addition of the candidate agent.

Any type of candidate agent may be tested by the discosed method for its potential in synergizing with the radiation to reduce growth of glioma cells. In some embodiments, the candidate agent is an agent that knockdowns expression of a lncRNA, particularly any of the lncGRS disclosed herein. In some embodiments, the candidate agent that knockdowns expression of a lncRNA is an miRNA, siRNA, ASO, or morpholino. In some embodiments, the lncRNA being knocked down comprises a nucleic acid sequence having at least about 70%, 75%, 80%, 85%, 90%, 95%, 96%, 97%, 98%, or 99% sequence identity to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 and SEQ ID NO: 9. In some embodiments, the candidate agent is an miRNA, siRNA, ASO, or morpholino that knocks down expression of a lncRNA comprising a nucleic acid sequence having at least about 70%, 75%, 80%, 85%, 90%, 95%, 96%, 97%, 98%, or 99% sequence identity to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 and SEQ ID NO: 9. In some embodiments, the lncRNA being knocked down comprises a nucleic acid sequence having at least about 70%, 75%, 80%, 85%, 90%, 95%, 96%, 97%, 98%, or 99% sequence identity to any of SEQ ID NO: 1. In some embodiments, the candidate agent is an miRNA, siRNA, ASO, or morpholino that knocks down expression of a lncRNA comprising a nucleic acid sequence having at least about 70%, 75%, 80%, 85%, 90%, 95%, 96%, 97%, 98%, or 99% sequence identity to SEQ ID NO: 1. In some embodiments, the candidate agent is an ASO that knocks down expression of a lncRNA comprising the nucleic acid sequence of SEQ ID NO: 1. In some embodiments, the candidate agent is an ASO comprising a nucleic acid sequence having at least about 70%, 75%, 80%, 85%, 90%, 95%, 96%, 97%, 98%, or 99% sequence identity to SEQ ID NO: 10 or SEQ ID NO: 11. In some embodiments, the candidate agent is an ASO comprising the nucleic acid sequence of SEQ ID NO: 10 or SEQ ID NO: 11.

In some embodiments, the candidate agent that knockdowns expression of a lncRNA comprises at least about 70%, 75%, 80%, 85%, 90%, 95%, 96%, 97%, 98%, 99% or 100% sequence identity to SEQ ID NO: 38, or a functional fragment thereof In some embodiments, the candidate agent that knockdowns expression of a lncRNA comprises at least about 70%, 75%, 80%, 85%, 90%, 95%, 96%, 97%, 98%, 99% or 100% sequence identity to SEQ ID NO: 39, or a functional fragment thereof. In some embodiments, the candidate agent that knockdowns expression of a lncRNA comprises at least about 70%, 75%, 80%, 85%, 90%, 95%, 96%, 97%, 98%, 99% or 100% sequence identity to SEQ ID NO: 40, or a functional fragment thereof. In some embodiments, the candidate agent that knockdowns expression of a lncRNA comprises at least about 70%, 75%, 80%, 85%, 90%, 95%, 96%, 97%, 98%, 99% or 100% sequence identity to SEQ ID NO: 41, or a functional fragment thereof In some embodiments, the candidate agent that knockdowns expression of a lncRNA comprises at least about 70%, 75%, 80%, 85%, 90%, 95%, 96%, 97%, 98%, 99% or 100% sequence identity to SEQ ID NO: 42, or a functional fragment thereof. In some embodiments, the candidate agent that knockdowns expression of a lncRNA comprises at least about 70%, 75%, 80%, 85%, 90%, 95%, 96%, 97%, 98%, 99% or 100% sequence identity to SEQ ID NO: 43, or a functional fragment thereof. In some embodiments, the candidate agent that knockdowns expression of a lncRNA comprises at least about 70%, 75%, 80%, 85%, 90%, 95%, 96%, 97%, 98%, 99% or 100% sequence identity to SEQ ID NO: 44, or a functional fragment thereof In some embodiments, the candidate agent that knockdowns expression of a lncRNA comprises at least about 70%, 75%, 80%, 85%, 90%, 95%, 96%, 97%, 98%, 99% or 100% sequence identity to SEQ ID NO: 45, or a functional fragment thereof. In some embodiments, the candidate agent that knockdowns expression of a lncRNA comprises at least about 70%, 75%, 80%, 85%, 90%, 95%, 96%, 97%, 98%, 99% or 100% sequence identity to SEQ ID NO: 46, or a functional fragment thereof.

In some embodiments, the candidate agent that knockdowns expression of a lncRNA is a fragment of any of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 and SEQ ID NO: 46. In some embodiments, such fragments of any of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 and SEQ ID NO: 46 comprise at least about 10, 11, 12, 13, 14, 15, 16, 17, 18, 19, 20 or more nucleotides. In some embodiments, such fragments of any of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 and SEQ ID NO: 46 comprise more than about 20, 30, 40, 50, 60, 70, 80, 90 or 100 nucleotides. In some embodiments, such fragments of any of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 and SEQ ID NO: 46 comprise from about 10 to about 200 nucleotides. In some embodiments, such fragments of any of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 and SEQ ID NO: 46 comprise from about 10 to about 150 nucleotides. In some embodiments, such fragments of any of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 and SEQ ID NO: 46 comprise from about 10 to about 100 nucleotides.

The level of radiation used to treat the human brain organoid model of malignant glioma in the disclosed method should be at a clinically therapeutic level. In some embodiments, the human brain organoid model of malignant glioma is treated with a total dose of radiation from about 1 Gy to about 12 Gy, from about 10 Gy to about 100 Gy, from about 20 Gy to about 90 Gy, from about 30 Gy to about 80 Gy, from about 40 Gy to about 70 Gy, from about 50 Gy to about 60 Gy, or from about 60 Gy to about 80 Gy. The radiation may be administered as a single dose or in fractions. When used in fractions, the radiation may be administered in about 2, 3, 4, 5, 6, 7, 8, 9 or 10 fractions. If necessary, the radiation may also be administered in more than 10 fractions. In some embodiments, the human brain organoid model of malignant glioma is treated with a fractionated radiation up to about 10, 20, 30, 40, 50, 60, 70, 80, 90 or 100. If necessary, the radiation may also be administered in less than 10 Gy or more than 100 Gy. In some embodiments, the human brain organoid model of malignant glioma is treated with a fractionated radiation up to about 24, 32, 40, 48 or 54 Gy. In some embodiments, the human brain organoid model of malignant glioma is treated with about 1, 2, 3, 4, 5, 6, 7, 8, 9 or 10 Gy fractionated radiation in about 2, 3, 4, 5, 6, 7, 8, 9 or 10 fractions.

### Systems

The disclosure also provides a system for detecting the presence, absence or quantity of any of the lncRNAs disclosed herein in a sample. The systems comprises: a) one or a plurality of nucleic acids complementary to, or substantially complementary to, one or a plurality of lncRNAs chosen from nucleic acid sequences comprising at least about 70%, 75%, 80%, 85%, 90%, 95%, 96%, 97%, 98%, 99% or 100% sequence identity to one or a plurality of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 and SEQ ID NO: 9; and b) a solid support onto to which the one or plurality of nucleic acids are immobilized. In some embodiments, the one or plurality of nucleic acids in the disclosed system comprises at least about 70%, 75%, 80%, 85%, 90%, 95%, 96%, 97%, 98%, 99% or 100% sequence identity to one or a plurality of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 and SEQ ID NO: 46, or a functional fragment thereof. In some embodiments, the one or plurality of nucleic acids in the disclosed system are fragments of any of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 and SEQ ID NO: 46, wherein the fragment comprises from about 10 to about 100 nucleotides. In some embodiments, the one or plurality of nucleic acids in the disclosed system are fragments of any of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 and SEQ ID NO: 46, wherein the fragment comprises from about 10 to about 20 nucleotides.

In some embodiments, the system of the disclosure comprises nucleic acids capable of detecting at least about two different lncRNAs in a sample. In some embodiments, the system of the disclosure comprises nucleic acids capable of detecting at least about three different lncRNAs in a sample. In some embodiments, the system of the disclosure comprises nucleic acids capable of detecting at least about four different lncRNAs in a sample. In some embodiments, the system of the disclosure comprises nucleic acids capable of detecting at least about five different lncRNAs in a sample. In some embodiments, the system of the disclosure comprises nucleic acids capable of detecting at least about six different lncRNAs in a sample. In some embodiments, the system of the disclosure comprises nucleic acids capable of detecting at least about seven different lncRNAs in a sample. In some embodiments, the system of the disclosure comprises nucleic acids capable of detecting at least about eight different lncRNAs in a sample. In some embodiments, the system of the disclosure comprises nucleic acids capable of detecting at least about nine different lncRNAs in a sample. In some embodiments, the system of the disclosure comprises nucleic acids capable of detecting at least about ten different lncRNAs in a sample. In some embodiments, the system of the disclosure comprises nucleic acids capable of detecting more than about ten different lncRNAs in a sample.

Any solid support known to one skilled in the art, including but not limited to microarray plate, glass slide, gel, or bead, can be used in the present disclosure. In some embodiments, the solid support is a microarray plate. In some embodiments, the solid support is glass slide. In some embodiments, the solid support is a gel. In some embodiments, the solid support is a bead.

### EXAMPLES

### Example 1

### CRISPRi-Based Radiation Modifier Screen Identifies Long Non-Coding RNA Therapeutic Targets in Glioma

Long noncoding RNAs (lncRNAs) exhibit highly cell type-specific expression and function, making this class of transcript attractive for targeted cancer therapy. However, the vast majority of lncRNAs have not been tested as potential therapeutic targets, particularly in the context of currently used cancer treatments. Malignant glioma is rapidly fatal, and ionizing radiation is part of the current standard-of-care used to slow tumor growth in both adult and pediatric patients.

We use CRISPR interference (CRISPRi) to screen 5689 lncRNA loci in human glioblastoma (GBM) cells, identifying 467 hits that modify cell growth in the presence of clinically relevant doses of fractionated radiation. 33 of these lncRNA hits sensitize cells to radiation, and based on their expression in adult and pediatric glioma, nine of these hits are prioritized as lncRNA Glioma Radiation Sensitizers (lncGRS). Knockdown of lncGRS-1, a primate-conserved, nuclear-enriched lncRNA, inhibits the growth and proliferation of primary adult and pediatric glioma cells, but not the viability of normal brain cells. Using human brain organoids comprised of mature neural cell types as a 3-dimensional tissue substrate to model the invasive growth of glioma, we find that antisense oligonucleotides targeting lncGRS-1 selectively decrease tumor growth and sensitize glioma cells to radiation therapy.

These studies identify lncGRS-1 as a glioma-specific therapeutic target and establish a generalizable approach to rapidly identify novel therapeutic targets in the vast non-coding genome to enhance radiation therapy.

1. Methods

i. Cell Culture and Radiation Treatment

U87, HeLa, NHA and HEK293T cells were grown in DMEM with 10% FBS and antibiotics/antimycotics. DIPG SF8628, GBM SF10360 and GBM 43 were cultured in N5 (Neurobasal-A (1×), N2 (1×), B27 supplement without vitamin A (1×), L-glutamine (1×)), 5% FBS, FGF and EGF (20 ng/mL each). SU-DIPG 24 and SU-DIPG 25 were cultured in TSM (Neurobasal-A (1×), DMEM/F12 (1×), HEPES (10 mM), sodium pyruvate (1 mM), MEM non-essential amino acids solution (0.1 mM), GlutaMax supplement (1×), antibiotic-antimycotic (1×)) with EGF (20 ng/mL), FGF-basic-154 (20 ng/mL), PDGF-AA (10 ng/mL), PDGF-BB (10 ng/mL) and Heparin (2 ug/mL). Proliferation was measured using a manual hemocytometer. Cell viability was measured using the CellTiter-Blue Cell Viability Assay (Promega, Madison, Wis.), and apoptosis was measured using the Apo-ONE Homogenous Caspase-3/7 Assay (Promega, Madison, Wis.). Radiation was delivered using a Cesium-137 irradiator with rotating platform, with cells treated while in suspension for the CRISPRi screen and after achieving adherence on plates for validation assays.

Determination of radiation synergy was calculated as follows. The null model of additive effects was determined by multiplying the mean decrease in cell proliferation caused by radiation treatment alone by the decrease in cell proliferation following gene knockdown. Synergistic effects were identified if the observed absolute decrease in cell proliferation following combination of lncRNA knockdown and radiation was significantly greater than the predicted decrease in cell proliferation based on the additive model (two tailed Student's t test).

ii. CRISPRi Screens

sgRNA library was derived from the CRISPRi Non-Coding Library (CRiNCL) [8], selecting sub-libraries that targeted all expressed lncRNAs in U87: Common+Cancer common+(U87, HEK293T)+U87 unique. sgRNAs were cloned into the library expression vector pCRISPRia-v2 [11,68] and lentivirus pools were generated as previously described [8]. U87-dCas9-KRAB cells were generated previously in [69]. Lentivirus libraries were infected in duplicate cultures, cultured for 2 days following infection, puromycin (1 mg/mL) selected for two days, and recovered for one day without puromycin. Cells were then cultured for 12 days at a minimum coverage of 1000×, starting at this “T0.” For the radiation modifier screen, doses of 2 Gy radiation were given at the following days: T0, T2, T4, T6, for a total of 8 Gy fractionated ionizing radiation. Genomic DNA was harvested from aliquots of ˜60M cells each from T0 and T12 and processed for sequencing as previously described [11,68]. Data processing and hit analysis was performed as described in [8], with the exception that neighbor hits were considered to be hits whose TSS's were within 1 kb of any protein coding gene TSS expressed in U87. Growth-only screen data for U87 was obtained from [8] and compared to the radiation screen data obtained in this study. For the identification of radiation sensitizers, the screen scores (defined as the average phenotype of the top three sgRNAs against a given gene multiplied by the negative log₁₀(Mann-Whitney-U p value) for that gene) for the radiation screen was compared to those of the growth screen for all genes targeted in both screens. Phenotype in these CRISPRi screens refer to the relative log 2 enrichment of barcodes in the final timepoint divided by the enrichment of barcodes at the initial timepoint, as has been previously described [8,11]. A screen score threshold of 5, which corresponded to an empirical false discovery rate of 0.25%, was applied to both screens, and hits with radiation scores greater than growth scores were retained. LncRNAs with evidence of expression in primary glioma cells were then identified as lncGRS's. Z standardized growth and radiation phenotypes were calculated as log 2 enrichment normalized by the standard deviation of negative control genes' phenotypes. Sensitizer score was defined as the ratio of the radiation modifier screen score in irradiated cells to the growth screen score in non-irradiated cells.

sgRNA validation and internally controlled growth assays was performed as described in [8], with the addition of 4 fractions of 2 Gy radiation starting two days following sgRNA infection, delivered every other day. Purified populations of sgRNA expressing cells were selected in 1 mg/mL puromycin for 3 days before assay. Internally controlled CRISPRi growth assays were performed as previously described, briefly, by partially infecting a population of cells stably expressing dCas9-KRAB with a fluorescently labeled sgRNA expression vector and tracking the sgRNA-infected population over time using flow cytometry relative to the non-infected population [8]. sgRNA protospacer sequences for individual knockdowns are listed in Table 2.

TABLE 2
CRISPRi sgRNA protospacer sequences used
for individual knockdown, qPCR
primers used, and ASO targeting sequences.

| | | |
|---|---|---|
| | | SEQ |
| | | ID |
| Description | Sequence | NO: |

sgRNA protospacer sequences

| | | |
|---|---|---|
| CTC-338M12.4 sgRNA 1 | GAAAGAGGGCGGCCCCGGAG | 18 |
| CTC-338M12.4 sgRNA 2 | GGAACCGGGCCGGGAGACGG | 19 |
| ERCC6L2 sgRNA 1 | GGCGCGGAAACCTCAGGCAA | 20 |
| ERCC6L2 sgRNA 2 | GGCGCTCGCCCCTTACGCAG | 21 |
| AC005624.2 sgRNA 1 | GCATCCCCGCGGGACCCCCA | 47 |
| AC005624.2 sgRNA 2 | GCTGTCTCGGCGCTGGGGAG | 48 |
| PDXDC2P sgRNA 1 | GGCGGGGCGGGGCCCTGTAA | 49 |
| PDXDC2P sgRNA 2 | GGTTCCGTGGGGGCCGCGAG | 50 |
| RP11-195F19.9 sgRNA 1 | GTGAGTGGAGGCGGGTTCTG | 51 |
| RP11-195F19.9 sgRNA 2 | GGTTCTGCGGAGGAGGAACC | 52 |
| SNHG1 sgRNA 1 | GATGAGAACGAATCTCCCCG | 53 |
| SNHG1 sgRNA 2 | GAAGACATTAGGTCAACTCA | 54 |
| RP1-122P22.2 sgRNA 1 | GGCCCGCGAGAGGTAAGGGG | 55 |
| RP1-122P22.2 sgRNA2 | GCCGCAGGTCCCGGAGGCGG | 56 |
| MIR210HG sgRNA 1 | GCTGAAGTTGGGCCGAGAGC | 57 |
| MIR210HG sgRNA 2 | GGTCGGGCCGGGGGGCGAGA | 58 |
| SNHG12 sgRNA 1 | GTAAGTCGACACCGGGAATG | 59 |
| SNHG12 sgRNA 2 | GGCCCCCTCGGCAGTGAGAG | 60 |
| RP11-339B21.10 sgRNA 1 | GCAGTGGAGGGGAGAGGTAT | 61 |
| RP11-339B21.10 sgRNA 2 | GTCTTCTCCCGGCCCCTCAA | 62 |

qPCR primers used

| | | |
|---|---|---|
| RPLPO-F orwardPrimer | TTCATTGTGGGAGCAGAC | 22 |
| RPLPO-ReversePrimer | CAGCAGTTTCTCCAGAGC | 23 |
| MALAT1 -F orwardPrimer | ATGCGAGTTGTTCTCCGTCT | 24 |
| MALAT1 -ReversePrimer | TATCTGCGGTTTCCTCAAGC | 25 |
| NEAT 1 -F orwardPrimer | GTGGCTGTTGGAGTCGGTAT | 26 |
| NEAT 1 -ReversePrimer | ATTCACTCCCCACCCTCTCT | 27 |
| U1-ForwardPrimer | ACGAAGGTGGTTTTCCCAG | 28 |
| U1-ReversePrimer | GTCCCCCACTACCACAAA | 29 |
| GAPDH-ForwardPrimer | GGGAAATTCAACGGCACAGT | 30 |
| GAPDH-ReversePrimer | AGATGGTGATGGGCTTCCC | 31 |
| lncGRS-1-ForwardPrimer | CTACCTGCCTTTTGGGGAAG | 32 |
| lncGRS-1-ReversePrimer | TTCGTCTTGACTCTGCAAGC | 33 |
| POLA1-F orwardPrimer | GCTATGTGGAAGATGCCGA | 34 |
| POLA1-ReversePrimer | TGTTCGGTTTTGTCACTGCG | 35 |
| TP53-F orwardPrimer | GCCCAACAACACCAGCTCCT | 36 |
| TP53-ReversePrimer | CCTGGGCATCCTTGAGTTCC | 37 |

ASO targeting sequences

| | | |
|---|---|---|
| CTC-338M12.4 ASO 1 | CCAACGGGAAAGGCGG | 10 |
| CTC-338M12.4 ASO 2 | CAGAAGAACGGTGGAC | 11 |
| TP53 ASO | CCAATGCGCCTGATTA | 16 |
| POLA1 ASO | CATTTAGTGTAAGCGG | 17 |

The sgRNA used in this experiment has the following sequence:

(SEQ ID NO: 63)
[protospacer]-GTTTAAGAGCTAAGCTGGAAAC
AGCATAGCAAGTTTAAATAAGGCTAGTCCGTTATCA
ACTTGAAAAAGTGGCACCGAGTCGGTGCTTTTTTT.

iii. RT-qPCR

RNA was harvested in TRIzol at 24 hours following ASO transfection, or in the case of CRISPRi, 72 hours following initiation of puromycin selection for sgRNA expression. RNA was purified using the Direct-zol MiniPrep or MicroPrep RNA purification kits (Zymo Research, Irvine, Calif.) with the on-column DNAse digestion step. cDNA was generated using the Transcriptor First Strand cDNA Synthesis Kit (Hoffmann-La Roche AG, Basel, Switzerland) and diluted 1:5 fold before proceeding to qPCR. qPCR was performed using the SYBR Green I Master Mix (Hoffmann-La Roche AG, Basel, Switzerland) on a LightCycler 480 instrument (Hoffmann-La Roche AG, Basel, Switzerland). qPCR primers are listed in Table 2.

iv. Subcellular Fractionation

Cells were plated in 15-cm dish and fractionated as previously described [70]. Briefly, 10 to 20 million cells were collected, washed with phosphate-buffered saline (PBS), and resuspended at 4×10⁷ cells/ml in buffer A (10 mM HEPES [pH 7.9], 10 mM KCl, 1.5 mM MgCl₂, 0.34 M sucrose, 10% glycerol, 1 mM dithiothreitol, and protease inhibitor cocktail [Boehringer Ingelheim, Ingelheim am Rhein, Germany]). Triton X-100 was added (0.1% final concentration), the cells were incubated on ice for 8 min, and nuclei (fraction P1) were collected by centrifugation (5 min, 1,300×g, 4° C.). The supernatant (fraction S1) was clarified by high-speed centrifugation (5 min, 20,000×g, 4° C.), and the supernatant (fraction S2) was collected. The P1 nuclei were washed once in buffer A and lysed for 30 min in buffer B (3 mM EDTA, 0.2 mM EGTA, 1 mM dithiothreitol, and protease inhibitor cocktail [Boehringer Ingelheim, Ingelheim am Rhein, Germany]), and insoluble chromatin (fraction P3) and soluble (fraction S3) fractions were separated by centrifugation (5 min, 1,700×g, 4° C.). The P3 fraction was washed once with buffer B. All the fractions obtained were resuspended in TRIzol for RNA extraction and qPCR was performed as described above.

v. Single-Molecule Fluorescence In Situ Hybridization (FISH)

In situ hybridization (ISH) was performed on cell lines and MBOs using the RNAscope 2.5 HD Assay—BROWN (Advanced Cell Diagnostics, Newark, Calif.). Probes targeting the lncGRS-1 transcript were used (RNAscope Probe Hs-CTC-338M12.4, catalog number 300031). ISH was performed following manufacturer's instructions.

vi. Western Blot

Cells were washed with PBS and lysed using RIPA buffer supplemented with HALT protease inhibitor (78429; ThermoFisher Scientific). The lysate was mixed (1:1) with 2× NuPAGE LDS Sample Buffer and ran on NuPAGE 10% Bis-Tris gel using NuPAGE MOPS SDS Running Buffer. Proteins were transferred from gel to Amersham Hybond PVDF membrane using NuPAGE Transfer Buffer with 10% methanol. The membrane was blocked for lhour in Odyssey Blocking Buffer and incubated overnight at 4° C. with primary antibodies—p21 (2947), 1:1000 and GAPDH_14C10(2118),1:1000 both from Cell Signaling Technologies. Following 3×10 minutes washes with PBS+0.1% Tween, the membrane was probed by the IRDye 800CW Goat anti-Rabbit IgG (926-32211). Images were captured using LiCOR Odyssey Infrared Imaging Systems and quantified using ImageJ. Western blot intensities were normalized by their respective loading controls and then normalized again to the mean of the negative control conditions.

vii. Immunohistochemistry for P53BP1 & Gamma-H2AX

Cells were plated in 8-well chambered slides (Thermo Scientific Nunc 154526) at a density of ˜10,000 cells/cm² and cultured overnight. Each chamber was transfected with either control ASO or lncGRS-1 ASO and then irradiated at 2 Gy. Cells were fixed 6 hours following radiation with 4% paraformaldehyde for 15 minutes at room temperature. They were further incubated in permeabilization reagent (0.1% Triton-X100 in TBS) for 15 minutes at room temperature. Cells were blocked in blocking solution (10% Normal Donkey Serum in TBS) for 1.5 hours in a humidified chamber. Cells were incubated in primary antibody solution anti-rabbit Phospho-53BP1 (Ser1778, Cell Signaling Technologies) at a 1:100 concentration and anti-rabbit Gamma H2AX (Ser139, Cell Signaling Technologies) at a 1:400 concentration. Plates were incubated overnight at 4° C. Cells were washed in PBST and secondary antibody and DAPI were added (Alexa 488 for P53BP1 and Alexa 594 for Gamma H2AX) at a 1:1000 concentration. Plates were incubated for 2 hours at room temperature in a humidified chamber away from light. Slides were washed and mounted with Aqua-PolyMount. Slides were left to dry in the dark at room temperature for 3 hours. Images were acquired at room temperature on an SP3 Leica Confocal microscope using 63× oil objective and processed using ImageJ.

viii. Flow Cytometry for Cell Cycle Analysis

Cells were transfected with ASOs as described below. After 72 hours, cells were pulsed with 33 μM bromodeoxyuridine (BrdU) for 20 min, and afterwards fixed in 70% ethanol. Cells were then stained with primary anti-BrdU antibody (Clone B44; BD Biosciences) for 1 hour, followed by 1 hour incubation with Alexa Fluor 488 anti-mouse IgG (Invitrogen). DNA was counterstained using 0.1 mg/ml propidium iodide supplemented with RNase for 1 hour at 37° C. Analysis was performed on a FACSCalibur using CellQuest (BD). Quantification and analysis of cell cycle profiles were performed using FlowJo (Tree Star, Inc).

ix. Antisense Oligonucleotides

Locked nucleic acid antisense oligonucleotides were designed using the Qiagen custom LNA oligonucleotides designer. ASOs were transfected at a final concentration of 50 nM using the Lipofectamine RNAiMAX Reagent using manufacturer's instructions (ThermoFisher Scientific, Waltham, Mass.). Three ASOs were tested and the top two based on knockdown efficiency were used for subsequent studies. For organoid transfection experiments, the ASO with the highest knockdown efficiency was used. For ASO penetration control studies, 5′-FAM labeled ASOs of Negative Control ASO A were used (Qiagen, Hilden, Germany). ASO target sequences are listed in Table 2.

x. Nanopore Direct RNA-Sequencing

Total RNA was isolated from U87 human glioblastoma cells using TRI Reagent Solution (ThermoFisher Scientific, Waltham, Mass.), followed by bead-based poly(A) selection. Approximately 750 ng of poly(A) RNA was used for dT adapter ligation, followed by reverse transcription, and additional ligation of motor adapter prior to loading onto the Oxford Nanopore Technologies (ONT, Oxford, United Kingdom) PromethION for sequencing. The ionic current trace for each poly(A) RNA strand was base called using the ONT Guppy algorithm.

xi. RNA-seq Sample Preparation and Data Analysis

For U87 radiation assays, RNA was harvested using TRIzol 48 hours following radiation treatment and purified using the Direct-zol MiniPrep RNA purification kits (Zymo Research, Irvine, Calif.) with the on-column DNAse digestion step. For ASO assays followed by RNA-seq, RNA was harvested at 24 hours following ASO transfection. RNA integrity was confirmed using the Agilent Bioanalyzer (Agilent Technologies, Santa Clara, Calif.). RNA-seq libraries were generated using TruSeq Stranded mRNA kit according to manufacturer's protocol (Illumina, San Diego, Calif.). cDNA was validated using the Agilent Bioanalyzer, Qubit 2.0 Fluorometer (Life Technologies, Carlsbad, Calif.), and ddPCR (Bio-Rad Laboratories, Hercules, Calif.). Cluster generation and sequencing was performed on a HiSeq 2500, using the paired end 100 read protocol.

Reads were aligned to the human genome (GRCh38) using the spliced read aligner HISAT2 v2.0.3 [71] against an index containing SNP and transcript information (genome_snp_tran). Quantification of Ensembl build 75 genes was carried out with featureCounts [72] using only uniquely mapped reads. Differential expression analysis was performed using DESeq2 [73] using the Wald test with an adjusted (multiple hypothesis corrected) p value threshold of 0.05 as threshold for differential expression. Complete linkage hierarchical clustering was performed using 1—Pearson correlation coefficients as the distance matrix, using only differentially expressed genes or differentially expressed lncRNAs. Gene ontology terms were obtained using Enrichr [74], taking gene names from the clusters of all upregulated or downregulated genes. Analysis was performed using R version 3.6.

xii. Mature Brain Organoids (MBO) and Tumor Co-Cultures

Generation of iAstrocytes

WTC11 human iPSCs were directed towards a cortical astrocyte fate as previously described [54,55]. Briefly, iPSCs were dissociated and reformed as embryoid bodies, dual SMAD inhibition was used to initiate neural induction using SB431542 and DMH1 (2 μM each) in defined media composed of DMEM F12, GlutaMax, sodium bicarbonate, sodium pyruvate, N-2 and B27 supplements. Once neuroepithelial cells were isolated, cultures were maintained in suspension for ˜180 days using defined media composition detailed above plus EGF and FGFb (10 ng/ml each) to drive proliferation and maturation into cortical astrocytes.

Generation of i³Neurons

WTC11 human iPSCs containing a transgenic doxycycline inducible cassette of NEUROG2 were induced into neurons as previously described [75]. Briefly, iPSCs were treated with doxycycline (2 μg/ml) in a defined media composed of DMEM F12, N-2 supplement, non-essential amino acids, and GlutaMax for 3 days. Populations were characterized as post-mitotic and expressing MAP2 and βIII-Tubulin to validate neuronal induction efficiency.

Generation of MBO Cultures

Combined iAstrocytes and i³Neuron mature organoids (AN-MBO) cultures were generated by combining iPSC derived iAstrocytes and iPSC derived i³Neuron at a ratio of 1:1 (iAstrocytes:i³Neurons), unless otherwise specified, in single cell suspension. Both iAstrocytes and i³Neurons were isogenic and derived from WTC11 human iPSCs. iAstrocyte mature organoids (A-MBO) were generated with iAstrocytes alone after 6-8 month generation time. Organoid spheres were generated by introducing 10,000-20,000 composite cells onto spheroid microplates (Corning). MBO cultures were prepared by combining desired cells types in single cell suspension and aliquoting the specified concentration across multiple wells of spheroid microplates (Corning). Microplates were then centrifuged at 300 g for 3 minutes. Organoids were allowed to coalesce for ˜48 hours prior to initiation of tumor cell seeding. Organoid cultures were maintained in AMO media—DMEM/F12, N2 supplement, B27 supplement, GlutaMax, antibiotic-antimycotic (Gibco) to 1× manufacturer's recommended final concentrations, and heparin (2 μg/mL, Sigma-Aldrich, St. Louis, Mo.).

DIPG Cell Seeding and Time Course

DIPG SF8628 cells labeled with lentiviral red fluorescent protein (RFP) were added as single cell suspension directly into 96 well plates, with each well containing a single pre-formed A-MBO, at a ratio of 1:5 (tumor cell:non-tumor cell). Tumor cells were seeded by pipetting directly into the culture media, and therefore only a small proportion of tumor cells invaded each organoid after seeding (presumably only those cells that landed directly on top of the organoid). 24 hours following seeding of DIPG cells onto A-MBOs, co-cultures were transfected with ASO (50 nM) and repeated once every 7 days. Co-cultures were maintained in AMO with EGF and FGFb (20 ng/ml each). Growth-arrested DIPG A-MBO co-cultures used for radiation does testing were maintained in AM0 without growth factors. Phase contrast and fluorescence images were obtained by focusing on a central Z-plane through the center of each organoid using a Leica DMI4000 B fluorescence microscope.

U87 Cell Seeding and Time Course

AN-MBOs comprised of 1:1 (iAstrocytes:i³Neurons) at 10,000 cells of each type were prepared before tumor cell seeding. AN-MBOs were allowed to mature for 2-3 weeks in AM0. GBM U87 cells labeled with lentiviral RFP (3500 cells per co-culture) were added as single cell suspension directly into 96 well plates, with each well containing a single pre-formed AN-MBO. 24 hours following seeding of GBM U87 cells onto AN-MBOs, co-cultures were transfected with ASO (50 nM) and repeated once every 7 days. Co-cultures were maintained in AM0 without growth factors. Phase contrast and fluorescence images were obtained by focusing on a central Z-plane through the center of each organoid using a Leica DMI4000 B fluorescence microscope.

Tumor Burden Quantification

2 dimensional fluorescence images acquired through the central focal plane of each organoid sphere were analyzed using ImageJ (Version 1.51m9, NIH). The 2 dimensional region of interest encompassing the MBO with tumor co-culture was calculated by manual selection of the sphere. The 2 dimensional size of the RFP+ tumor infiltrate within the interior of each organoid was quantified by thresholding the fluorescence intensity to exclude non-tumor infiltrated regions and tumor-free organoids, and excluding any RFP+ regions that fall outside the spherical boundaries of the total organoid co-culture surface area. Thresholding was performed in an unbiased manner with the same color intensity applied across replicates and time-points.

Organoid Preparation for Confocal Imaging

MBOs were fixed using 4% paraformaldehyde and incubated at 4° C. for 60 minutes. Organoids were then washed three times with PBS at room temperature, allowing 5 minutes incubation per wash. Organoids were then incubated for 24 hours at 4° C. in PBS with 30% (wt/vol) sucrose. Organoids were embedded in disposable base molds (Fisherbrand #22363552) using embedding solution (1:1, OCT:30% sucrose solution). Embedded organoids were frozen and sliced using a cryotome producing 15 μm sections and mounted on microscopy slides. Imaging was performed using a Leica TCS SP5 X confocal microscope and analyzed in imageJ.

2. Results

i. A CRISPRi Platform for Radiotherapy Sensitization in a Glioma Cell Culture Model

To systematically identify lncRNAs as potential therapeutic targets that sensitize malignant glioma to radiotherapy, we developed a radiation modifier screen using CRISPRi for gene knockdown. CRISPRi represses transcription via the recruitment of catalytically “dead” Cas9 protein fused to the KRAB repressor (dCas9-KRAB), which is targeted to transcriptional start sites (TSS) by a single guide RNA (sgRNA) [11,31,32]. For the screen, we used a workhorse GBM cell line (U87) engineered to stably express dCas9-KRAB (U87-dCas9-KRAB) to identify hits for subsequent study in patient-derived cultures of pediatric and adult forms of malignant glioma.

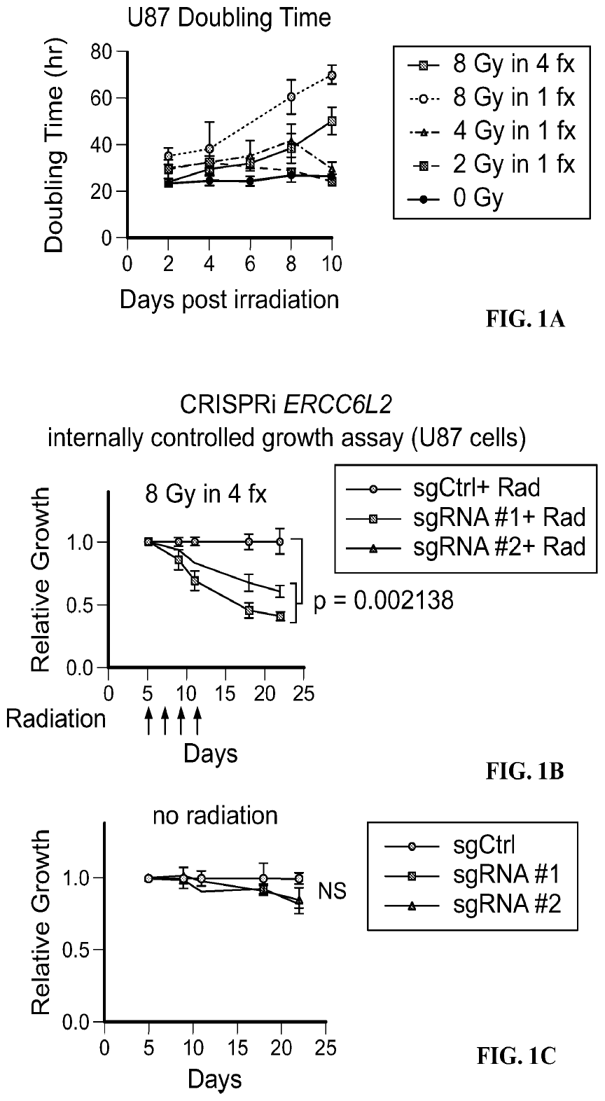

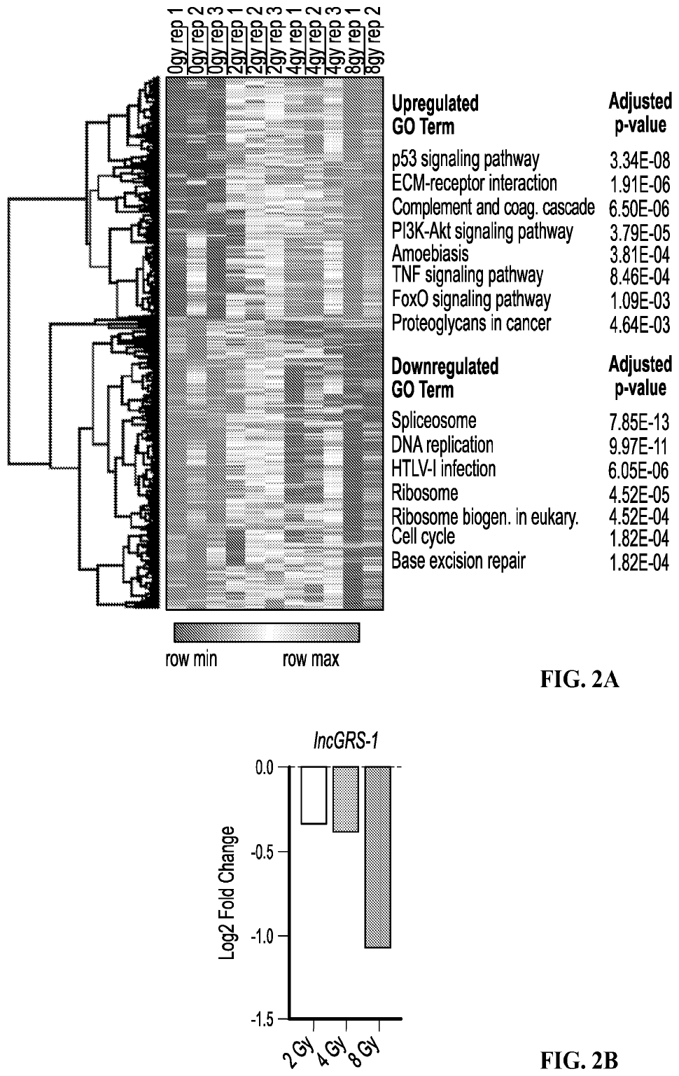

First, a radiation dose and delivery schedule in U87-dCas9-KRAB cells that enables the discovery of radiation-effect modifiers was determined. For the treatment of human GBM patients, the total radiation dose is typically delivered in ˜2 Gy daily fractions [18,19]. When 2 Gy was delivered to U87-dCas9-KRAB cells as a single dose, cell proliferation transiently decreased but returned to normal after 8-10 days (FIG. 1A). A single dose of 4 or 8 Gy had correspondingly stronger and more prolonged inhibitory effects upon cell proliferation (FIG. 1A), and RNA-seq analysis revealed dose dependent gene expression changes including induction of the p53 pathway and repression of DNA replication (FIG. 2A). However, these higher single doses exceed those typically used for patients with malignant glioma. Thus, a total radiation dose of 8 Gy delivered in 2 Gy fractions given every other day (8 Gy in 4 fractions) was also tested. This 8 Gy fractionated dose increased the cell doubling time by approximately 2-fold, which is an effect size approximating LD₅₀ (FIG. 1A), which allows optimal discovery of both synergistic and buffering screen hits [33], while still utilizing a clinically relevant fractional dose of 2 Gy.

Whether U87-dCas9-KRAB cells treated with 8 Gy fractionated radiation can reveal radiation sensitization effects with CRISPRi gene targeting was next tested. The inhibition of DNA repair pathways is known to potentiate the therapeutic effects of radiation in cancer cells [34,35]. In internally controlled growth assays [8,11], two distinct CRISPRi sgRNAs targeting the DNA damage repair gene ERCC6L2 [36,37] reduced cell growth in U87 cells treated with 8 Gy fractionated radiation (FIG. 1B). Without radiation treatment, CRISPRi targeting of ERCC6L2 did not reduce cell growth (FIG. 1C). Thus, CRISPRi targeting of the DNA repair gene ERCC6L2 produces a strong radiation sensitization effect in this in vitro model of fractionated radiotherapy.

ii. CRISPRi Screen Identifies lncRNA Glioma Radiation Sensitizer (lncGRS) Hits

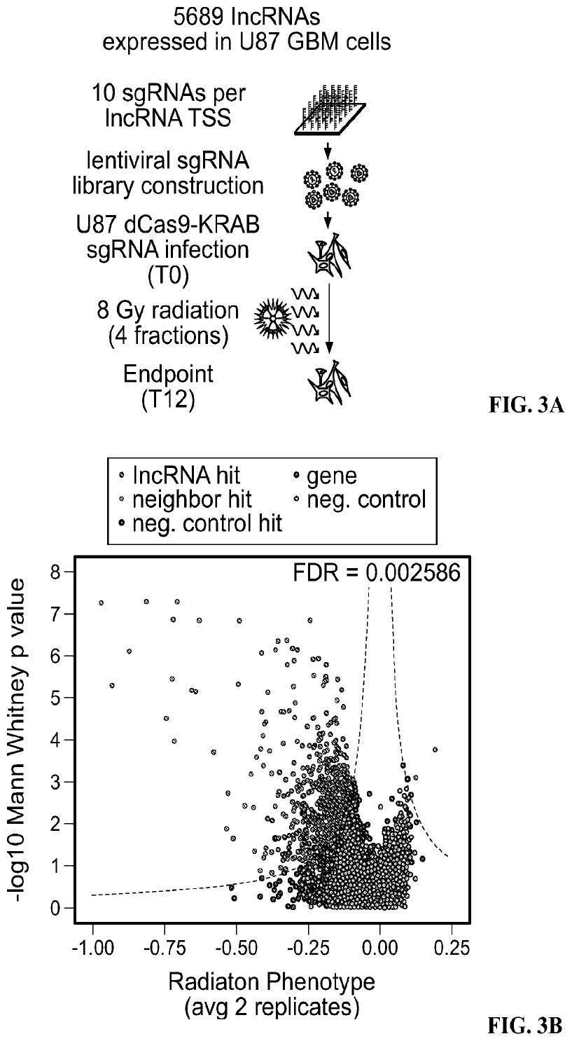

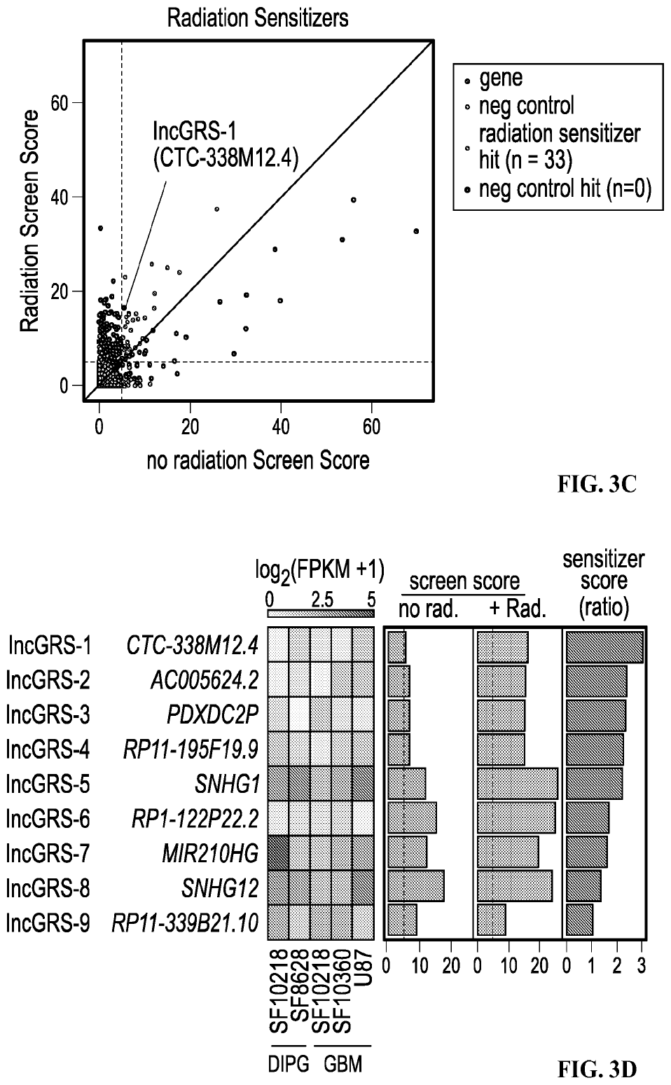

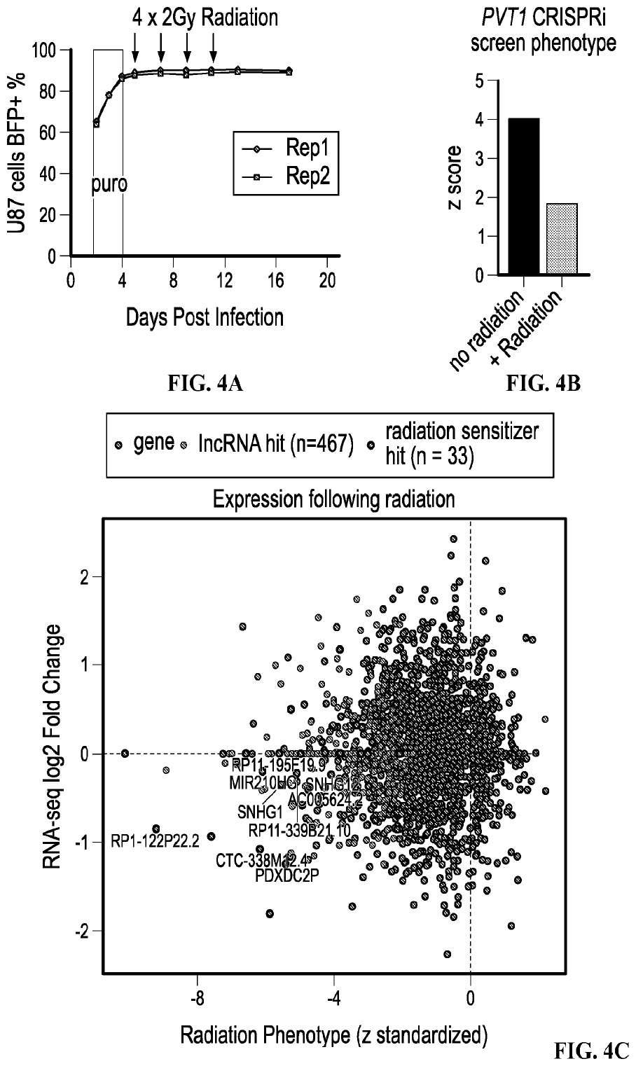

Although disruption of known DNA repair pathways is an intriguing avenue for novel therapeutics, accumulation of unrepaired DNA damage may paradoxically increase cancer risk [38,39]. The human genome produces thousands of lncRNAs that represent a large set of novel potential therapeutic targets. The function of 5689 lncRNAs expressed in human glioma were screened by leveraging a CRISPRi Non-Coding Library [8], selecting 10 sgRNAs for each lncRNA TSS and cloning this pool of 56,890 sgRNAs into lentiviral vectors along with 1202 non-targeting control sgRNAs (FIG. 3A). This lentiviral sgRNA library was used to infect two replicates of U87-dCas9-KRAB cultures, selected for infected cells with puromycin, treated cultures with 8 Gy fractionated radiation, then continued cell propagation for a total of 12 days. The proportion of sgRNA positive cells remained stable throughout the screen, indicating that CRISPRi targeting does not exhibit non-specific toxicity even after radiation treatment (FIG. 4A).

Targeted next generation sequencing of sgRNA barcodes performed at the beginning and end of the screen (˜7.1 cell doublings) identified 652 loci that modified cell growth and proliferation in the presence of radiation as compared to the effect of radiation alone (FDR=0.25%). 185 of these lncRNA TSS hits were within 1 kb of an expressed protein-coding gene and therefore conservatively labeled as “neighbor hits,” with the 1 kb window around the TSS being based on analyses of CRISPRi mechanism and maximum effective distance for knockdown [8,11]. These neighbor hits were removed from further analysis, leaving 467 “lncRNA hits” that modified the propagation of radiation-treated U87 cells (FIG. 3B). Interestingly, CRISPRi-mediated knockdown of the lncRNA PVT1 protected cells against the effect of radiation (FIG. 4B), consistent with PVT1 acting as a negative regulator of MYC [40]. The other 466 lncRNA hits negatively affected cell culture growth when combined with radiation.

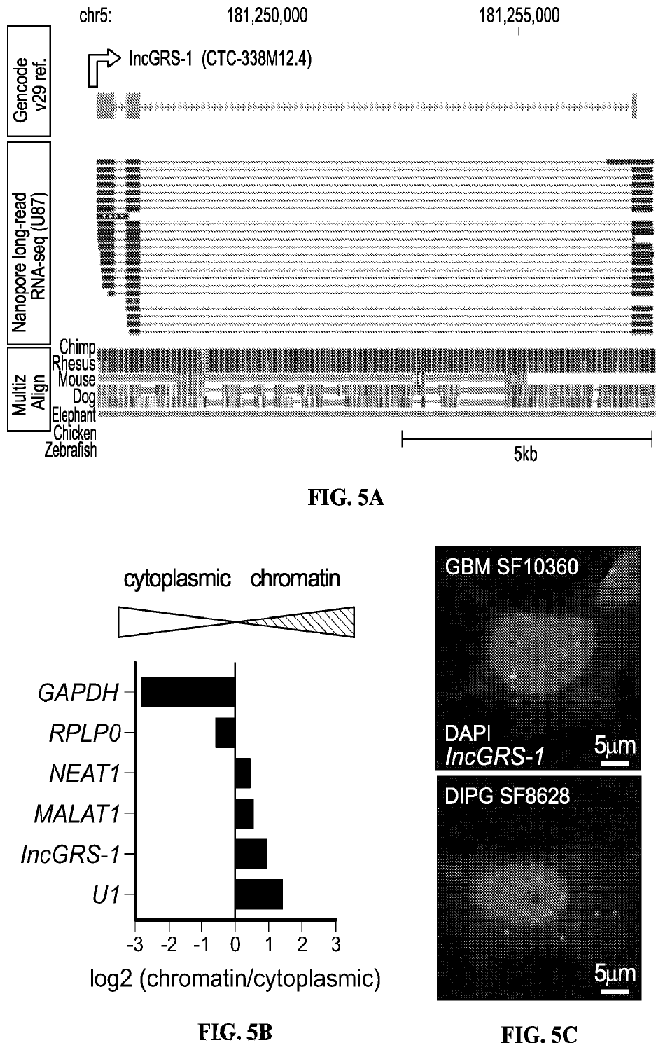

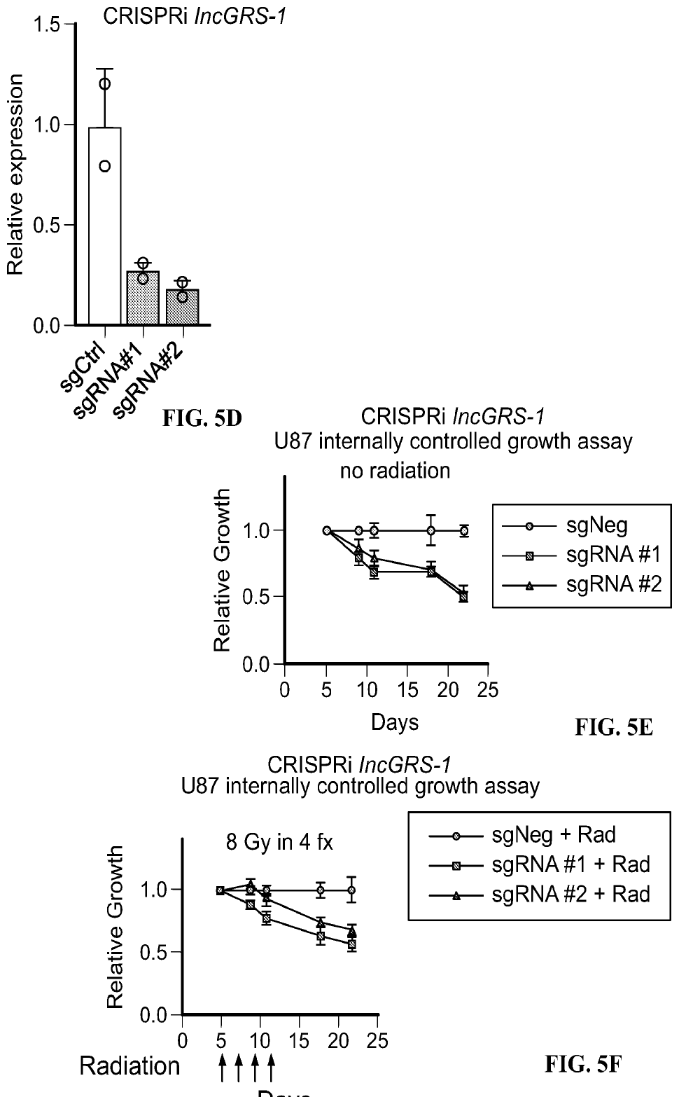

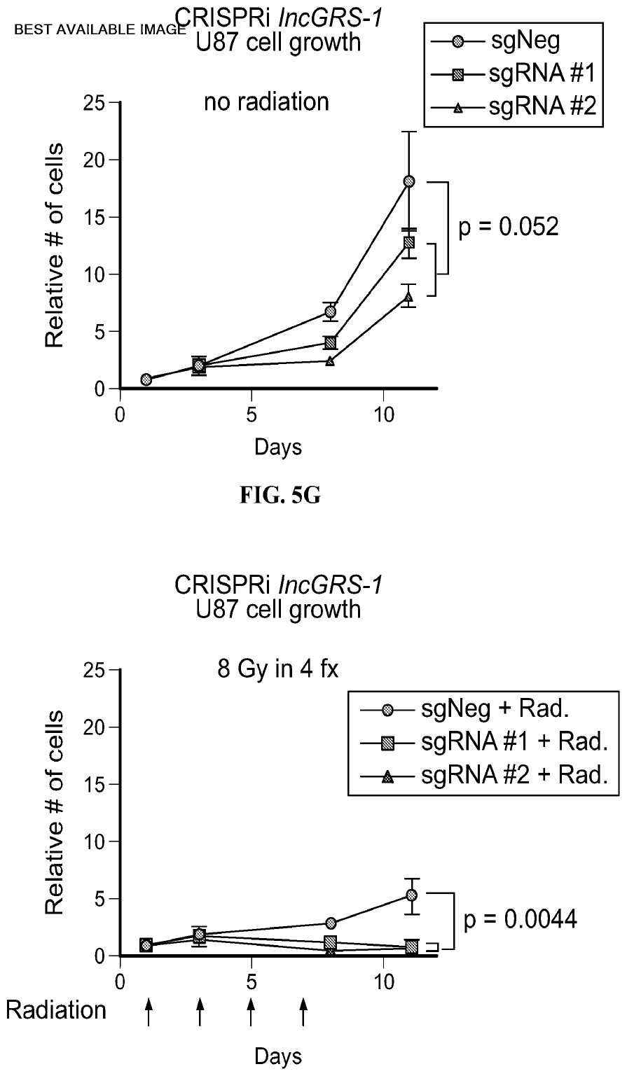

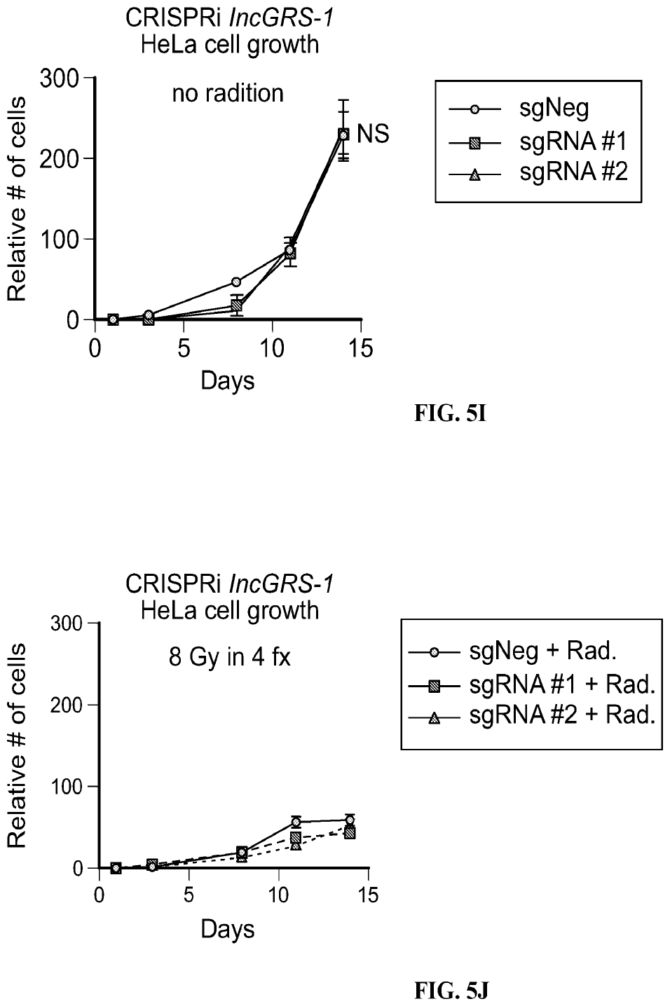

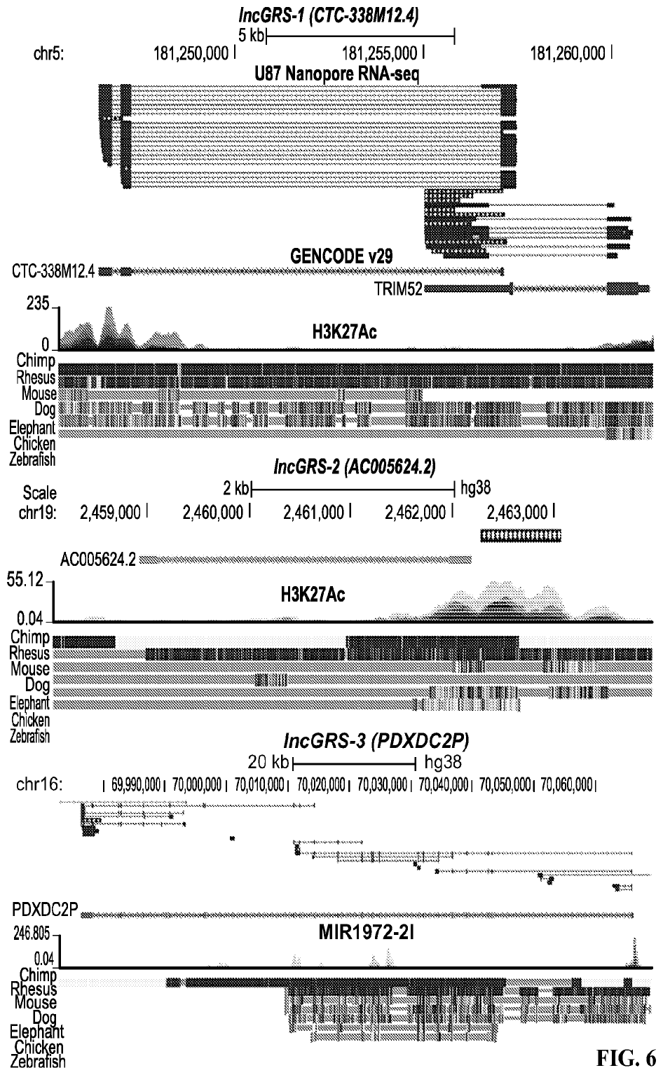

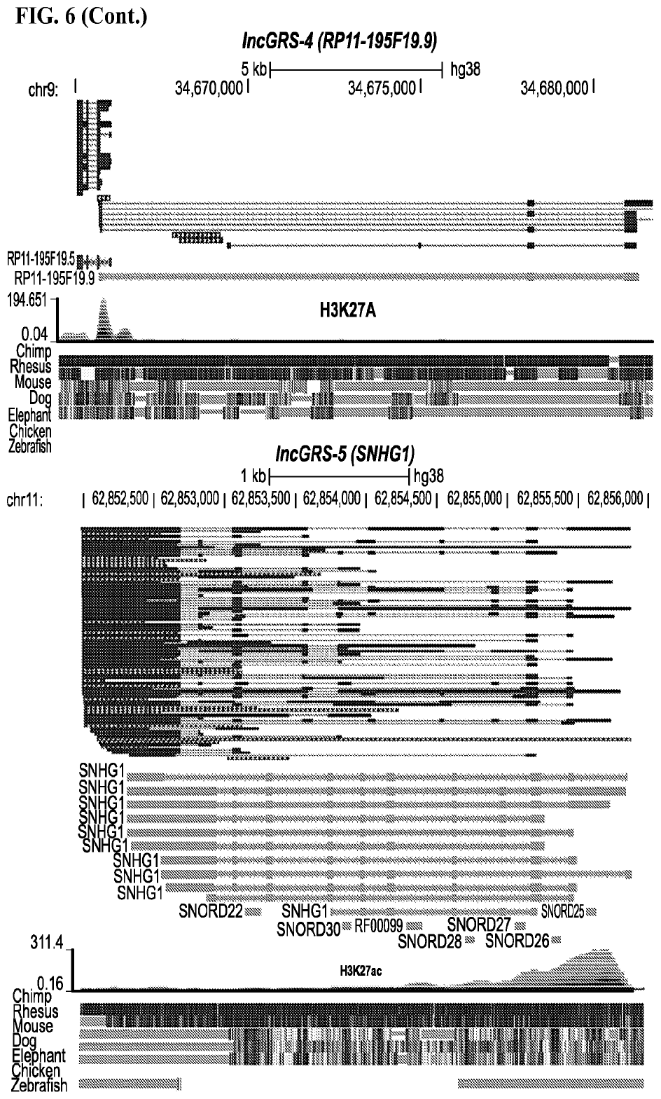

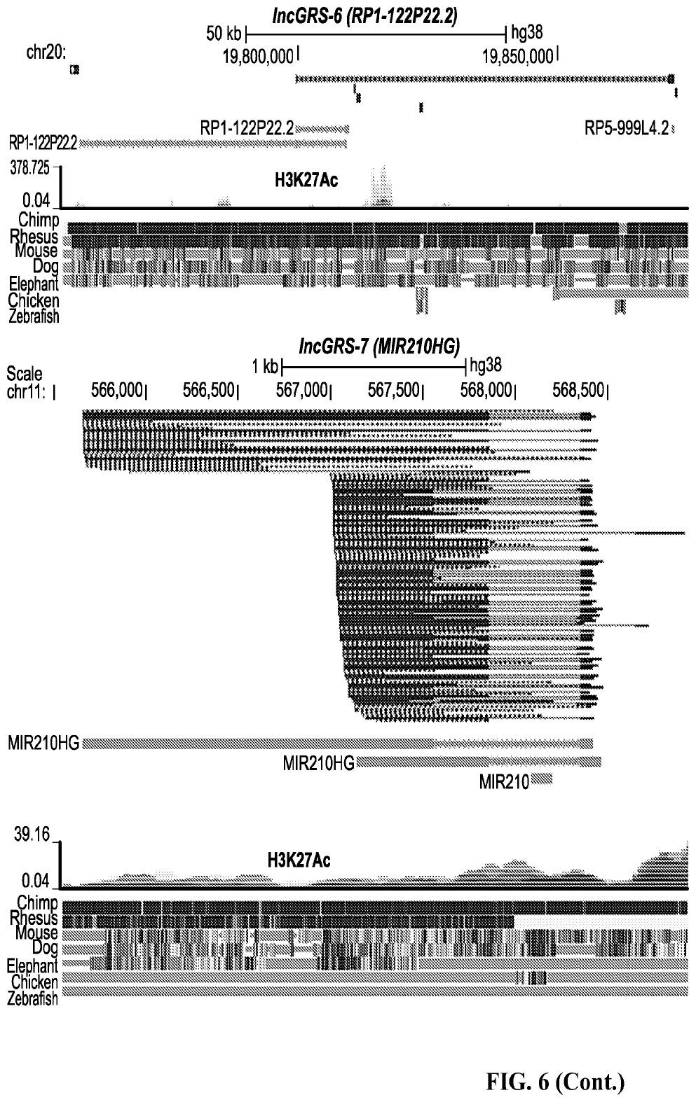

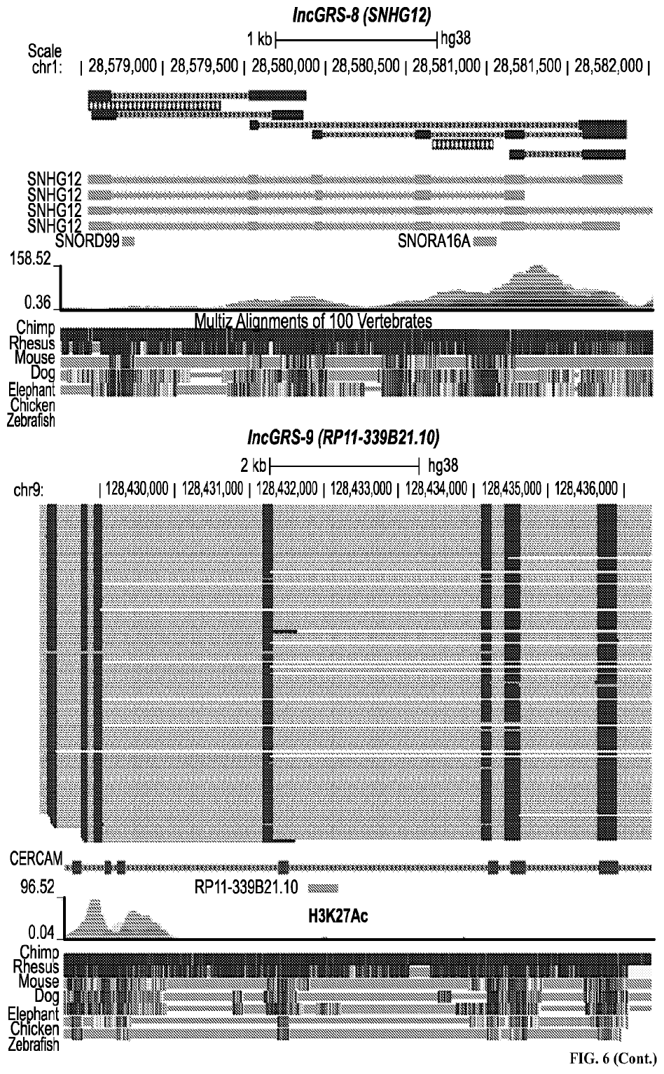

To identify lncRNA hits that sensitized cells to the effect of radiation, the screen scores (defined as the average phenotype of the top three sgRNAs against a given gene multiplied by the negative log₁₀(Mann-Whitney-U p value) for that gene) for the radiation modifier screen were compared with the growth screen scores from U87 cells that were not irradiated [8,11]. 33 hits identified in both screens had screen scores that were greater in the radiation modifier screen (FIG. 3C). Interestingly, the expression of these 33 sensitizer lncRNA hits tended to be downregulated following exposure to 8 Gy of radiation (Pearson R=0.36), which was not observed for the larger set of 467 hits (Pearson R=0.079) (FIG. 4C), suggesting that transcriptional repression following radiation may be a common feature of lncRNA radiosensitizers. Nine of these 33 sensitizer hits were expressed in a panel of various malignant glioma cells including both adult GBM (U87, SF10360, SF10281) and pediatric DIPG (SF8628, SF10218). These nine hits were ranked by a “sensitizer score,” which is defined as the ratio of the radiation modifier screen score in irradiated cells to the growth screen score in non-irradiated cells, and denoted these genes as lncRNA Glioma Radiation Sensitizers (lncGRS) 1 to 9 (FIG. 3D). To more accurately survey the transcript structure(s) of these hits, long-read single molecule native RNA sequencing was performed using the Oxford Nanopore PromethION and defined transcript variants and splice boundaries of lncGRS-1 through 9 (FIG. 5A and FIG. 6).

iii. CRISPRi-Mediated Knockdown of lncGRS-1 Synergizes with Fractionated Radiation

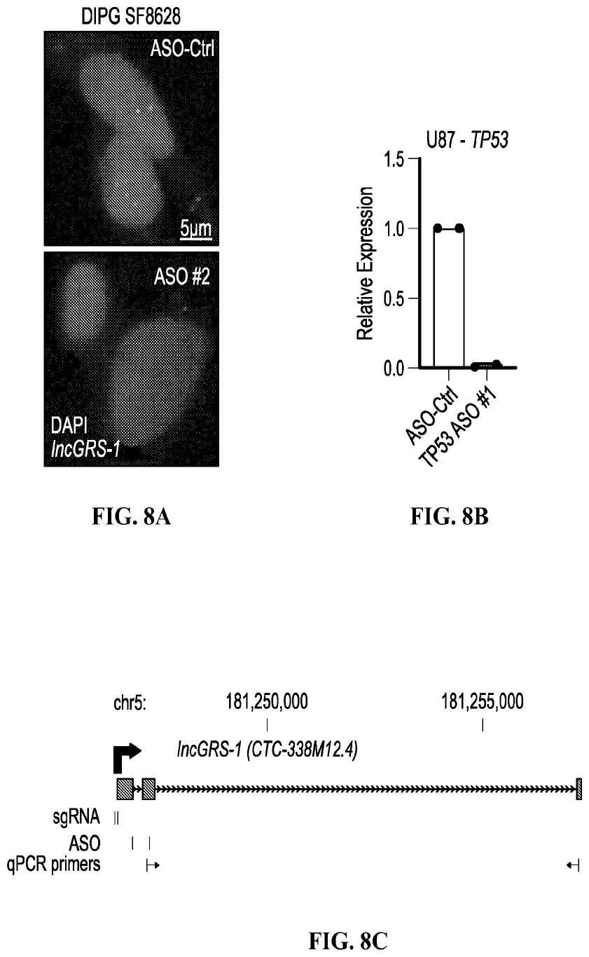

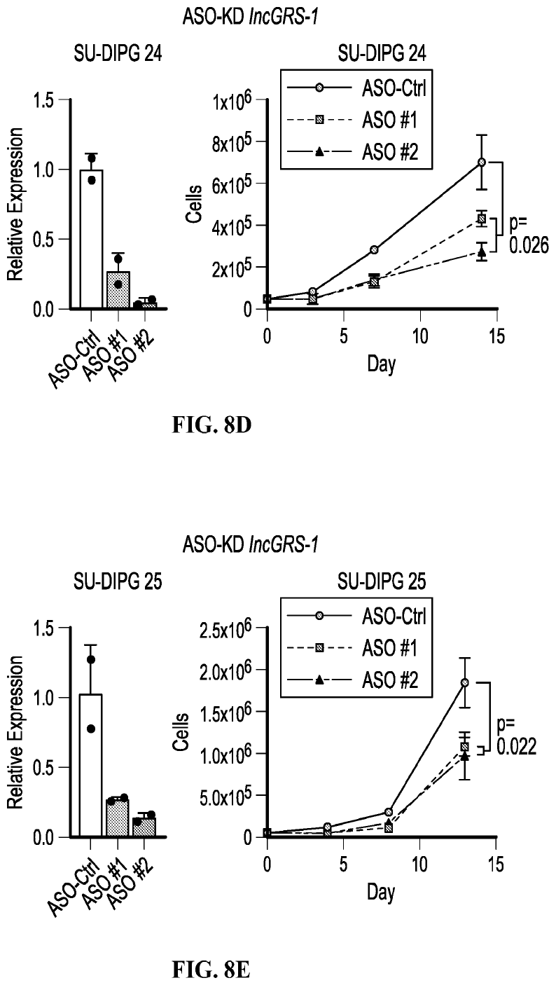

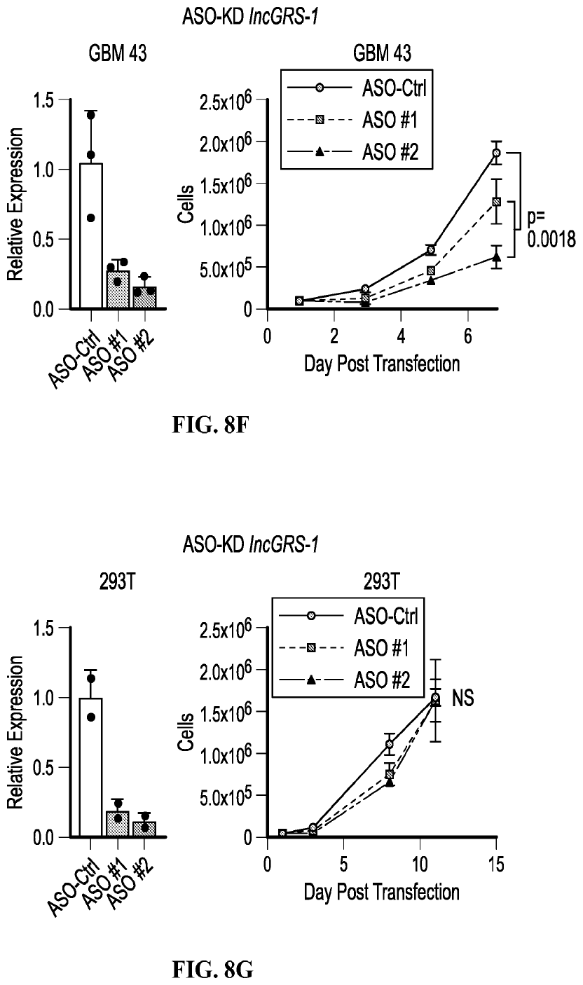

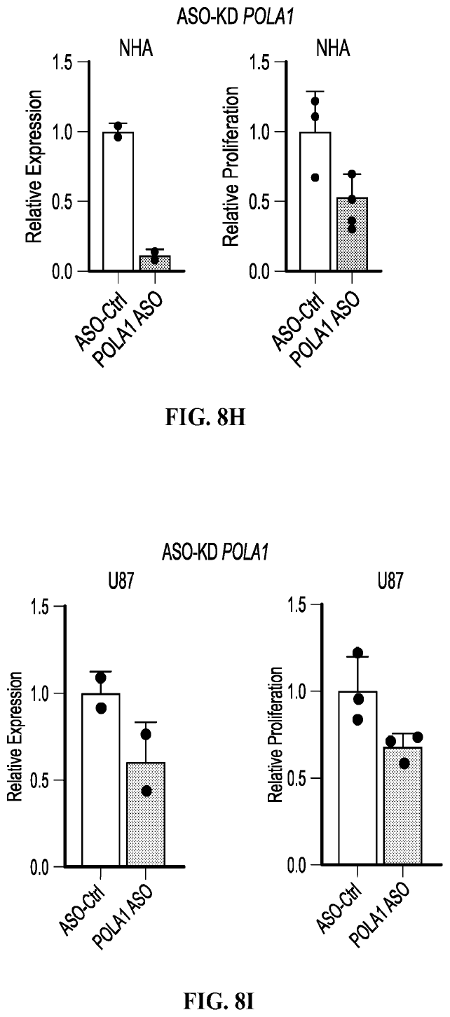

lncGRS-1 encodes spliced, poly-adenylated transcripts (687 to 1013 bp) from chromosome 5 (FIG. 5A and FIG. 6). Although previously annotated (CTC-338M12.4) [41], this lncRNA's biological function was not known. Cell fractionation analysis (FIG. 5B) and in situ hybridization studies (FIG. 5C and FIG. 8A) revealed lncGRS-1 to be a nuclear-enriched transcript in glioma cells, including those of patient-derived, primary glioma cultures. Furthermore, lncGRS-1 transcription is downregulated following radiation (FIG. 2B).

In internally controlled growth assays of U87 cell growth, CRISPRi-mediated knockdown of lncGRS-1 with two different, individual sgRNAs (FIG. 5D) reduced cell proliferation by ˜48% in the absence of radiation (FIG. 5E). Treatment of cells with 8 Gy fractionated radiation alone decreased cell proliferation (FIG. 1A), and lncGRS-1 knockdown further reduced cell proliferation by ˜37% relative to radiation alone (FIG. 5F). U87-dCas9-KRAB cells that express sgRNA against lncGRS-1 or non-targeting control sgRNAs were purified and the growth of these cultures with and without 8 Gy fractionated radiation was studied. Without radiation, sgRNAs against lncGRS-1 reduced cell proliferation by 42% (FIG. 5G), and radiation alone reduced the proliferation of cells expressing control sgRNAs by 71% (FIG. 5G and FIG. 5H). When combined, lncGRS-1 knockdown and radiation treatment resulted in a pronounced 95% decrease in proliferation (FIG. 5H), indicating synergy of the two treatments, as the predicted decrease in cell proliferation from an additive effect model is only 83% (p=0.0052). Interestingly, while radiation decreased proliferation of HeLa cervical cancer cells by 74%, consistent with known radiosensitivity of cervical cancer [42], the combination of CRISPRi-mediated lncGRS-1 knockdown with radiation was indistinguishable from radiation alone (FIG. 5I and FIG. 5J), suggesting that lncGRS-1 is a cell type-specific radiation sensitizer target.

iv. CRISPRi- and Antisense Oligonucleotide (ASO)-Mediated lncGRS-1 Knockdown Inhibits the Growth of Glioma Cells from Both Adult and Pediatric Patients

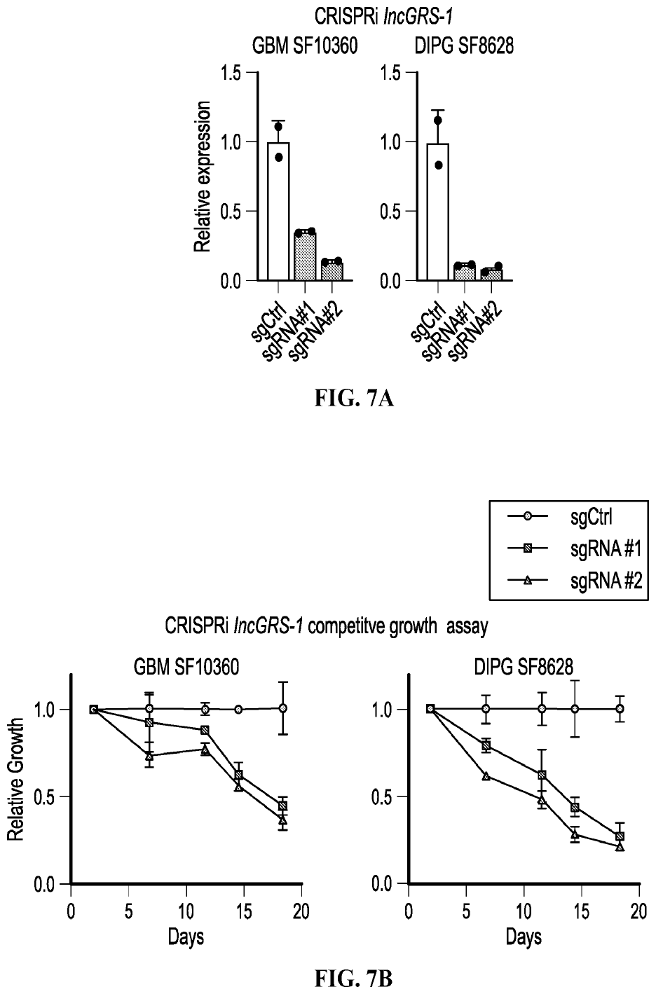

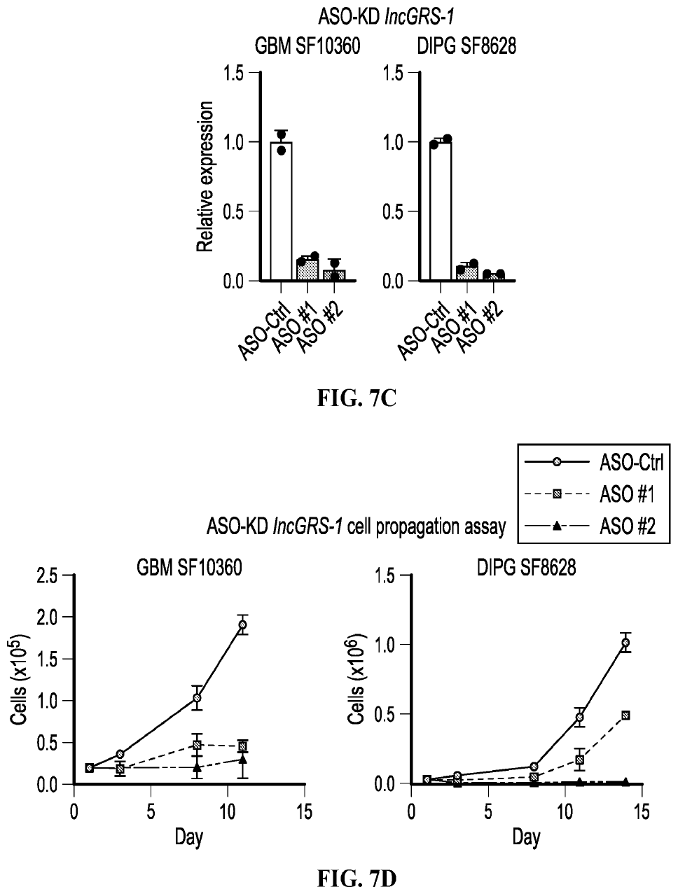

Validating lncGRS-1 as a potential therapeutic target in patient-derived cultures of malignant glioma was next performed. Human adult GBM (SF10360) and pediatric DIPG (SF8628) cells stably expressing dCas9-KRAB were generated and the effect of CRISPRi-mediated lncGRS-1 knockdown was studied in internally controlled growth assays. In both GBM SF10360 and DIPG SF8628 cells, lncGRS-1 knockdown produced growth inhibition (FIG. 7A and FIG. 7B) similar to that observed in U87 cultures. Thus, CRISPRi targeting of lncGRS-1 slows the growth of GBM cell lines and primary malignant glioma cells in culture.

Antisense oligonucleotides (ASOs) degrade complementary RNAs via a ribonuclease H-based mechanism, are effective for the knockdown of nuclear lncRNAs [43], and are currently used to treat human CNS diseases [44,45]. To confirm the ability of ASOs to efficiently deplete gene expression, we used ASOs to knockdown TP53 (p53) in U87 cells and observed ˜99% knockdown efficiency (FIG. 8B). Locked nucleic acid ASOs against lncGRS-1 were then designed (FIG. 8C; Table 2) and tested for efficacy in patient-derived glioma cell cultures. Two different ASOs against lncGRS-1 produced a mean knockdown of 89% in patient-derived GBM SF10360 cells and 93% in patient-derived DIPG SF8628 cells (FIG. 7C). Cell proliferation was decreased by an average of 80% across both ASOs in both SF10360 and SF8628 by 11 days post transfection (FIG. 7D). ASO-mediated knockdown of lncGRS-1 also decreased the proliferation of patient-derived DIPG cultures SU-DIPG 24, SU-DIPG 25 (FIG. 8D and FIG. 8E), and GBM 43 (FIG. 8F). Thus, ASOs targeting lncGRS-1 inhibit the growth of multiple human patient-derived cultures of malignant glioma.

v. ASO-Mediated Knockdown of lncGRS-1 is not Toxic to Human Astrocytes

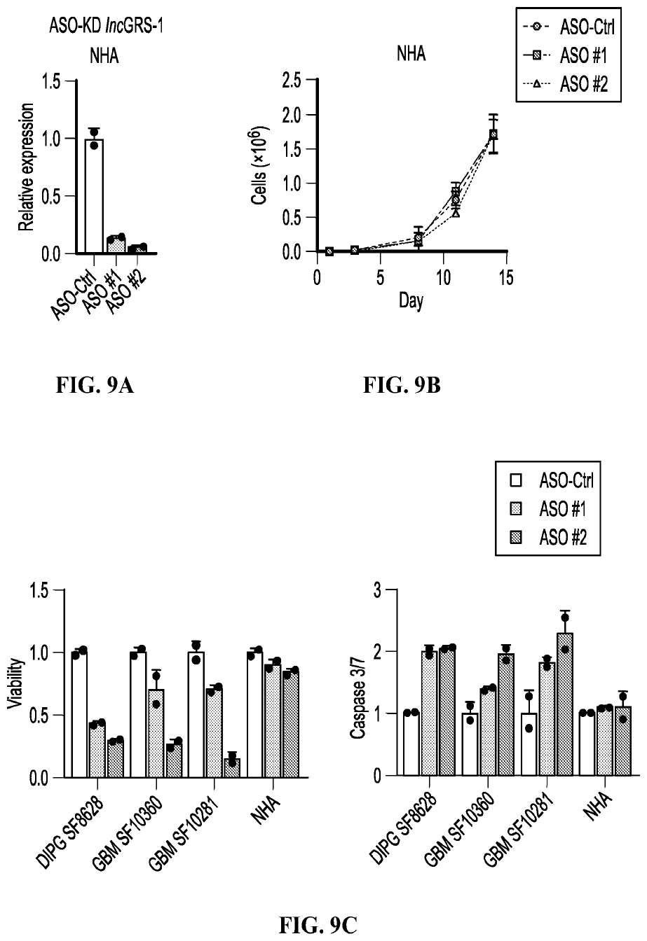

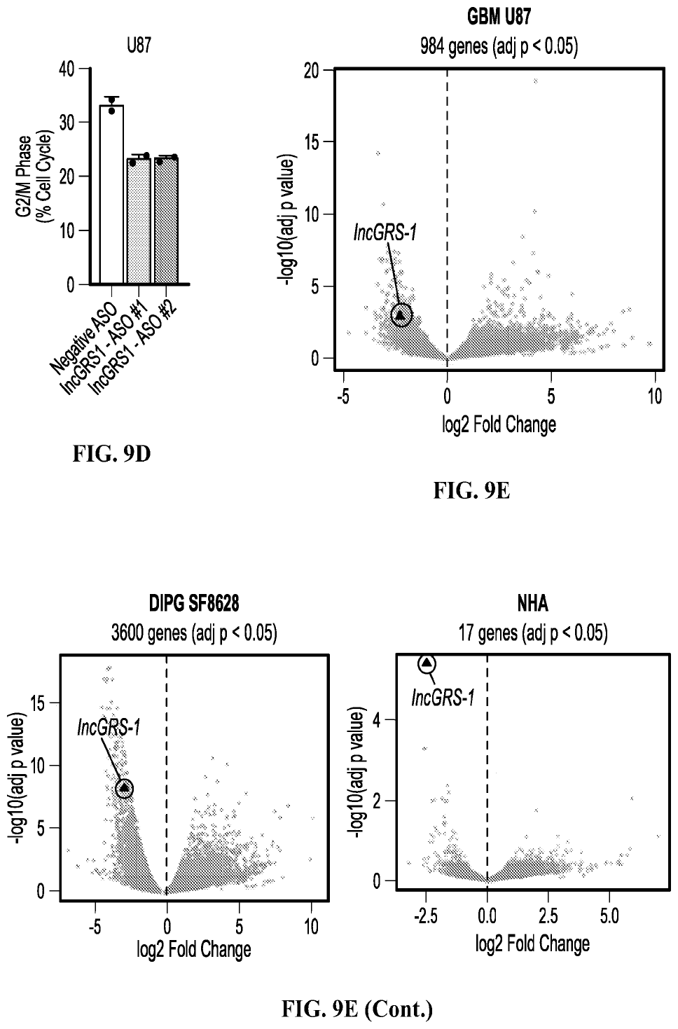

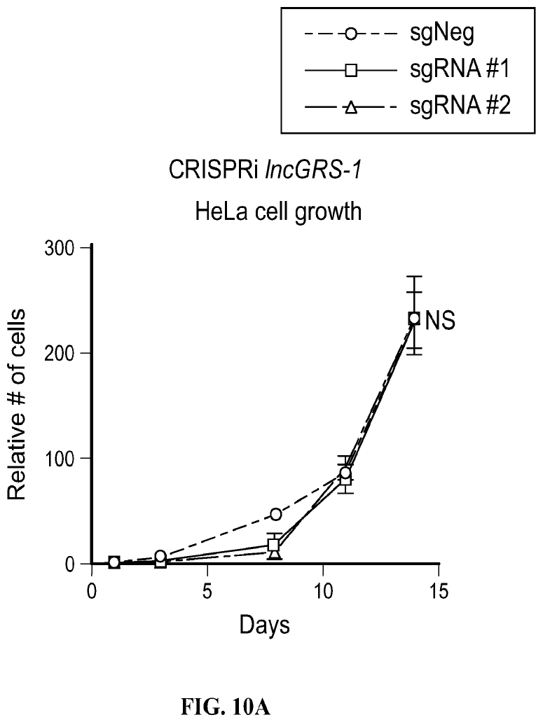

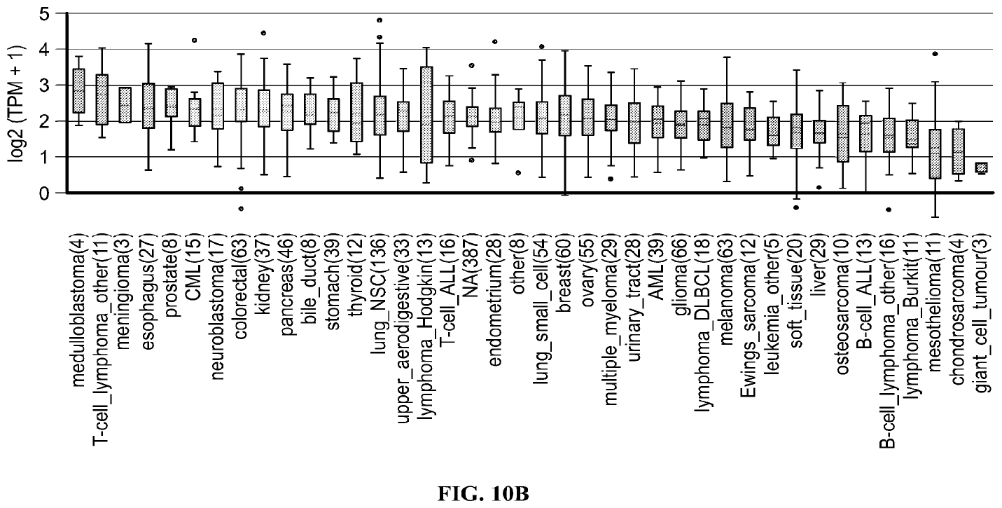

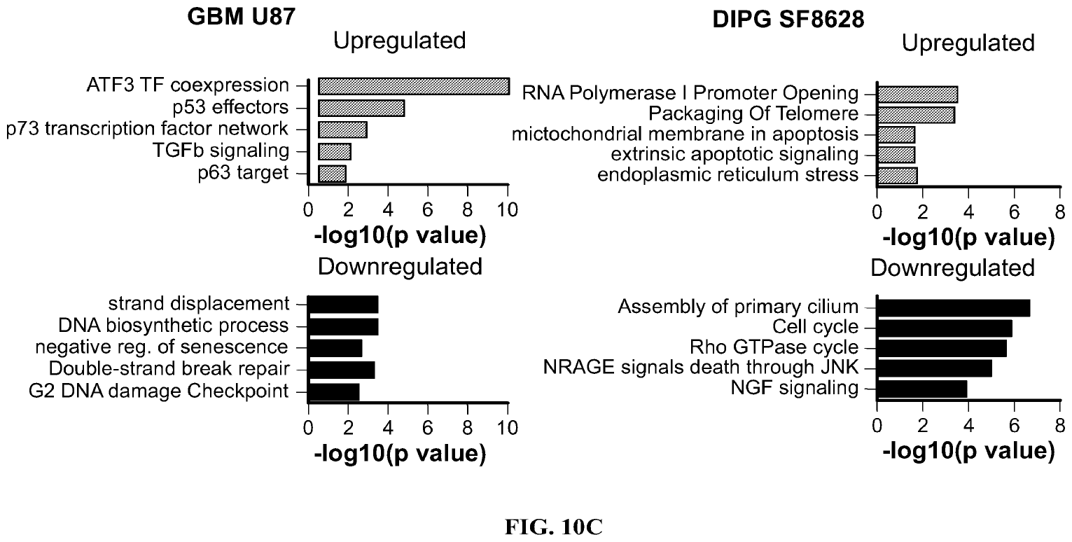

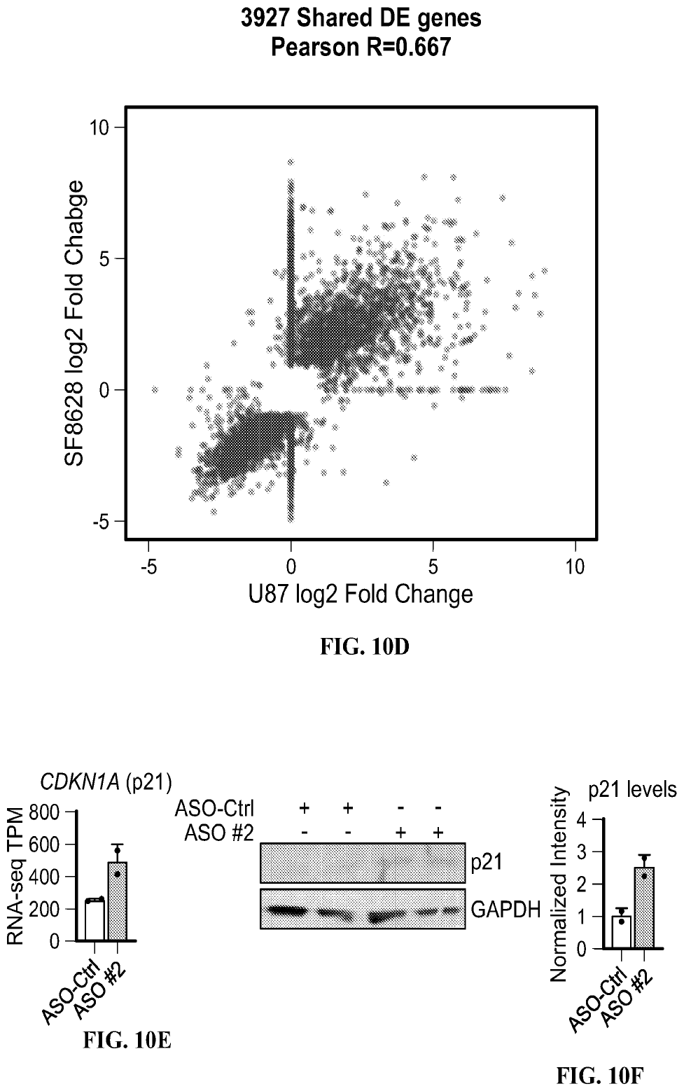

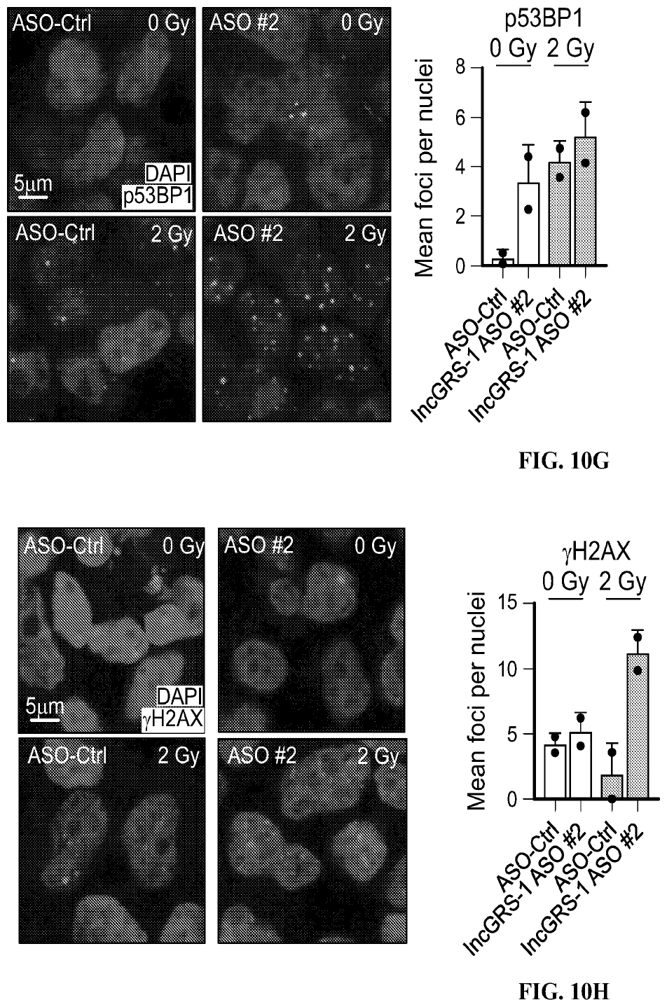

Current standard-of-care treatments for glioma are limited by toxicity to normal tissues [23]. Consistent with lncRNAs having exquisitely cell type-specific essential function [8], ASO-mediated knockdown of lncGRS-1 did not affect the proliferation of the kidney derived HEK293T cell line (FIG. 8G). Astrocytes are the most numerous cell type of the human brain [46] and are phenotypically related to subpopulations of malignant glioma [47,48]. Thus, the effect of lncGRS-1 knockdown in cultures of normal human astrocytes (NHA) [49] was next investigated. Knockdown of essential gene POLA1 (DNA polymerase alpha 1) resulted in decreased proliferation of NHA cells, in addition to U87 GBM cells (FIG. 8H-8i). While ASO-mediated lncGRS-1 knockdown was robustly achieved in NHA (FIG. 9A), cell proliferation was not reduced (FIG. 9B). Moreover, in NHA, measures of cell viability and apoptosis were not changed by lncGRS-1 knockdown, whereas in patient-derived glioma cells, viability was decreased and apoptosis was increased (FIG. 9C). Finally, CRISPRi-mediated knockdown of lncGRS-1 with the same two sgRNAs used for knockdown in U87 cells did not result in decreased proliferation in HeLa cervical cancer cells stably expressing dCas9-KRAB (FIG. 10A), despite these cells expressing lncGRS-1 at a robust level (TPM=4.38) amongst the CCLE compendium of cancer cell lines (FIG. 10B) [42].

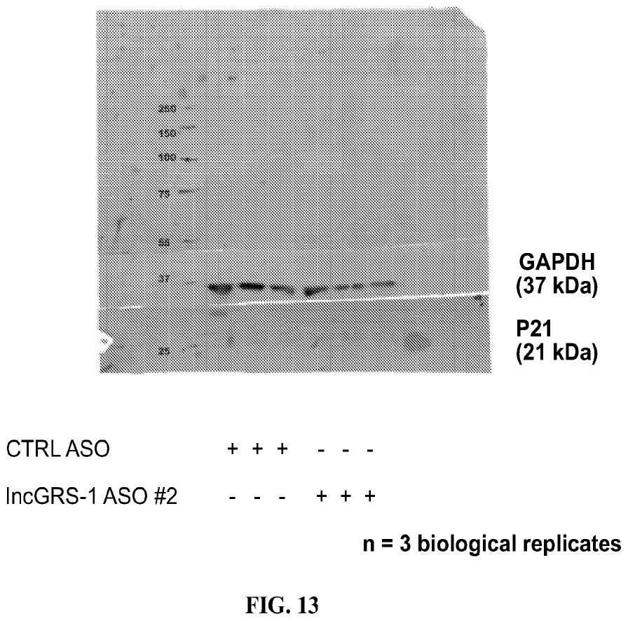

To investigate the glioma-specific phenotype of lncGRS-1, RNA-seq analysis following ASO-mediated lncGRS-1 knockdown was performed. In GBM (U87), DIPG (SF8628), and NHA cells, lncGRS-1 expression was decreased by 80-88% 24 hours after ASO treatment (FIG. 9E). Consistent with the changes in viability and apoptosis observed with lncGRS-1 knockdown, both GBM and DIPG cells exhibited transcriptome-wide differential gene expression (984 and 3600 genes adj. p<0.05, respectively; FIG. 9E). Upregulated genes in U87 and SF8628 were enriched for p53 signaling and apoptosis, while downregulated genes were enriched for cell cycle and DNA damage response (FIG. 10C). Despite different gene ontology terms, differentially expressed genes between U87 and SF8628 cells were positively correlated following knockdown of lncGRS-1 (Pearson R=0.667; FIG. 10D). Consistent with these genome-wide changes, levels of CDKN1A (p21) increased at the level of both mRNA and protein following lncGRS-1 knockdown in U87 cells (FIG. 10E-10F, FIG. 13). These cells also exhibited decreased proportions of G2/M phase in population level flow cytometry (FIG. 9D). Interestingly, while lncGRS-1 knockdown resulted in increased p53BP1 foci in U87 cells in the absence of radiation, γH2AX foci induced by radiation were potentiated by knockdown of lncGRS-1, yet lncGRS-1 knockdown in the absence of radiation did not generate additional γH2AX foci (FIG. 10G-10H). Remarkably, while ASOs were effective for lncGRS-1 knockdown in NHA, such genome-wide changes to the transcriptome were not observed in these non-tumorigenic cells (FIG. 9E).

vi. ASOs Targeting lncGRS-1 Decrease Glioma Tumor Growth in Mature Brain Organoids (MBOs)

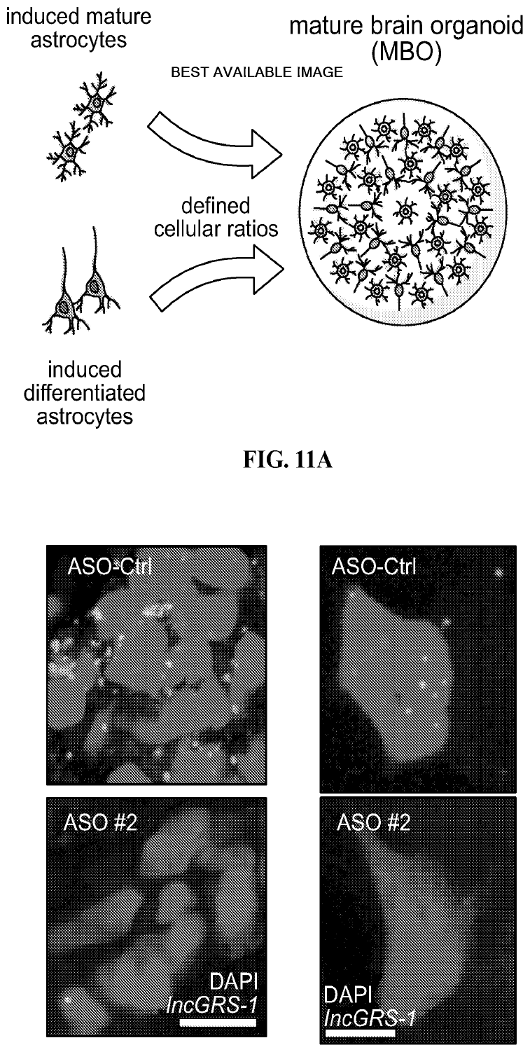

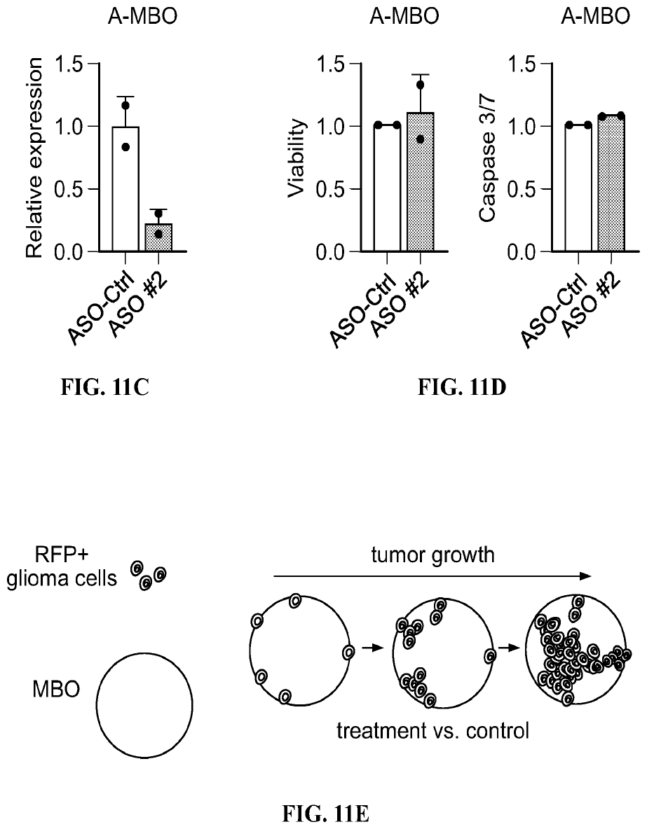

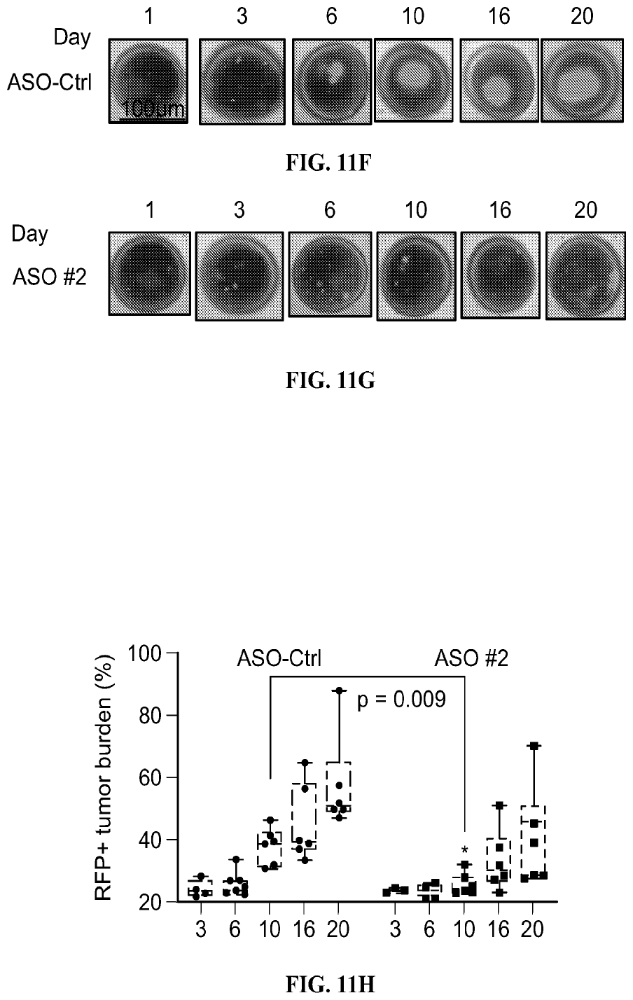

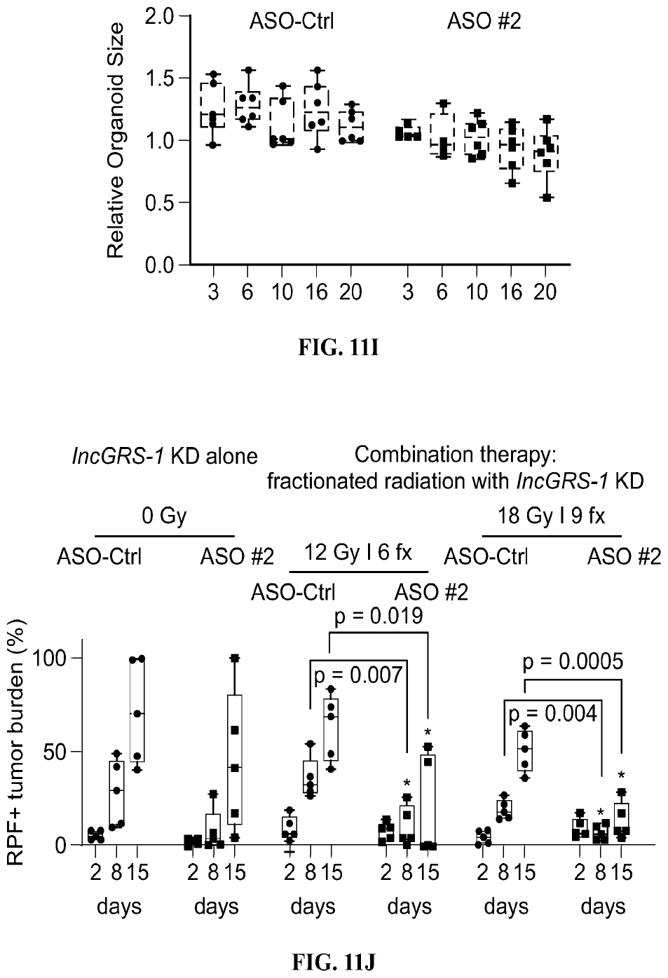

While the genomic sequences of lncGRS-1 are conserved across primates, orthologs of this lncRNA do not exist in lower vertebrates such as mice, chicken, and zebrafish (FIG. 5A and FIG. 6). Therefore, evaluating the potential toxicity of lncGRS-1 knockdown in normal postnatal brain necessitates an alternative to xenograft mouse models. Recent studies have shown that embryonic cerebral organoids can serve as 3D tissue “hosts” for the growth of human glioma cells [30]. However, such embryonic brain organoids are comprised mostly of immature, proliferative neural progenitors and contain very few astrocytes [50-52], which are a type of glia essential to neuronal viability and function [53]. Thus, “mature” human brain organoids (MBOs) that more closely reflect the differentiated cellular state of the postnatal human brain (FIG. 11A) were generated.

Astrocytes are the most abundant cell type of the adult human brain [53]. Pure populations of mature human astrocytes from human iPSCs (iAstrocytes) were generated using a protocol that allows maturation for at least six months [54,55]. Using an isogenic iPSC (WTC11) clone that carries an inducible Neurogenin2 (NGN2) transgene, we also generated homogenous cultures of mature cortical neurons (iNeurons) with NGN2 induction [54,56]. MBOs can be formed from iAstrocytes and iNeurons by mixing and co-culture of these cell types in defined numbers and ratios (from a 1:1 ratio to solely iAstrocytes or iNeurons) (FIG. 11A) [55].

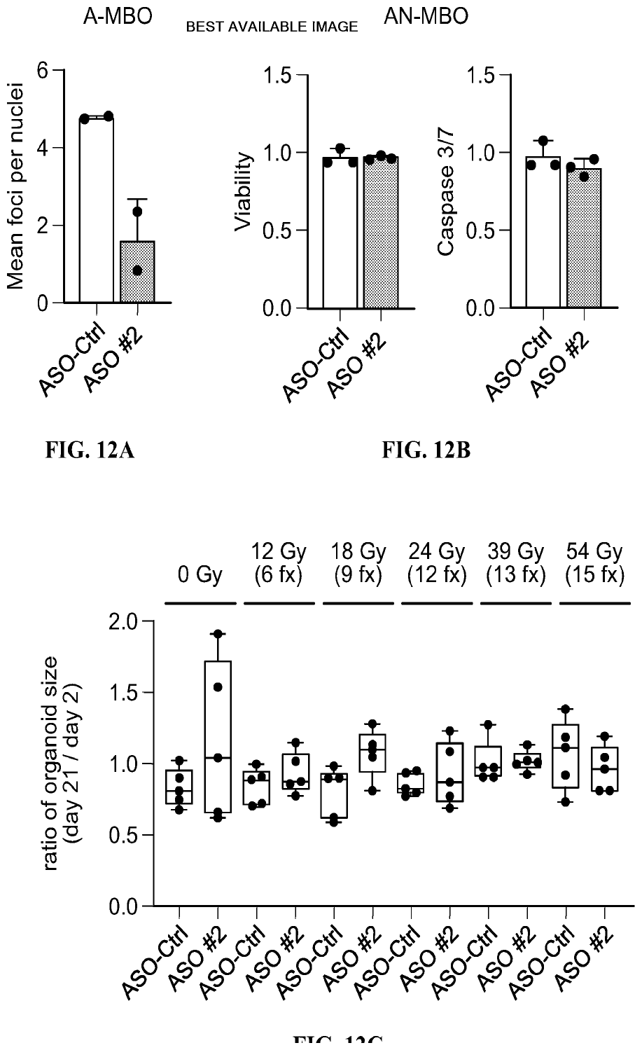

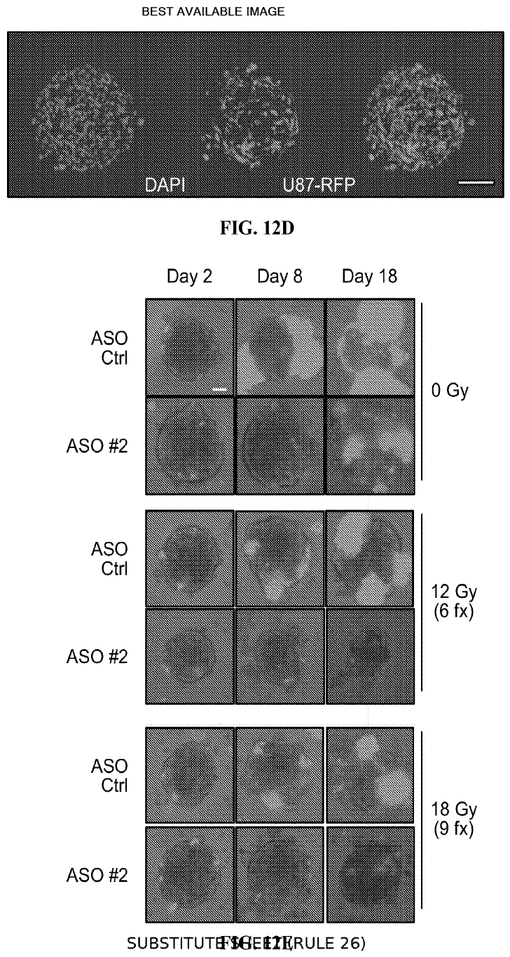

To build upon the studies of NHAs in 2D culture, MBOs assembled from iAstrocytes (A-MBOS) were first studied. Unlike astrocytes in 2D culture, iAstrocytes in MBOs become highly ramified and develop complex structures similar to those observed in human brain tissue [55]. Transfection of A-MBOs with ASOs against lncGRS-1 was effective for its knockdown as assessed by both in situ hybridization, demonstrating depletion of lncGRS-1 within nuclei (FIG. 11B), and RT-qPCR (FIG. 11C, FIG. 12A). Similar to our results from NHAs in 2D culture (FIG. 9A-9C), ASO-mediated lncGRS-1 knockdown in A-MBOs did not reduce organoid viability or increase apoptosis (FIG. 11D).

A-MBOs were next used as the “host” for human glioma tumor growth (FIG. 11E). After DIPG SF8628 cells labeled with red fluorescent protein (RFP) were seeded to the surface of these MBOs, RFP+ tumors grew progressively larger within the organoid tissue in an invasive manner, when individually monitored using serial microscopic imaging (FIG. 11F). For treatment of a human CNS disease, ASOs are administered weekly directly into the cerebrospinal space [57]. Mimicking this dosing schedule, ASOs were added into the organoid culture media every 7 days. In each organoid, the growth of RFP+ tumors was prospectively imaged (FIG. 11F and FIG. 11G), and tumor burden was estimated by quantification of the RFP signal. ASOs against lncGRS-1 reduced DIPG tumor growth as compared to non-targeting, negative control ASOs (FIG. 11H). Consistent with our results of organoid viability and apoptosis without infiltrated tumor cells (FIG. 11D), the overall size of the host brain organoid was not changed by lncGRS-1 knockdown (FIG. 11I).

The MBO-glioma model was further developed by using MBOs assembled from both iAstrocytes and i³Neurons (AN-MBOs) and also including radiation therapy. ASO-mediated knockdown of lncGRS-1 did not affect the viability of AN-MBOs (FIG. 12B). Furthermore, treatment of AN-MBOs with clinically therapeutic levels of fractionated radiation (total dose up to 54 Gy), with or without lncGRS-1 knockdown, did not affect the overall size of organoids over a period of 3 weeks (FIG. 12C). Using this AN-MBO model of radiotherapy, whether lncGRS-1 knockdown sensitizes glioma cells to the therapeutic effects of radiation was investigated. RFP labeled U87 cells were seeded to the surface of individual AN-MBOs isolated across multiple wells of a 96-well dish, and tumors grew invasively (FIG. 12D) and progressively larger when treated with negative control ASOs and no radiation when tracked using serial microscopic imaging (FIG. 12E). As expected, radiation treatment alone (control ASOs with increasing doses of radiation) exhibited a trend of tumor inhibition (FIG. 11J). Notably, when radiation (18 Gy in 9 fractions, or 12 Gy in 6 fractions) was combined with ASO-mediated lncGRS-1 knockdown, the tumor burden was significantly lower than that observed with radiation alone (FIG. 11J and FIG. 12E). Thus, ASO-mediated knockdown of lncGRS-1 sensitizes malignant glioma to the therapeutic effects of radiation in this 3D model of tumor growth.

3. Discussion

In this study, a CRISPRi-based pooled screening platform was developed to discover novel non-coding therapeutic targets in human malignant glioma that can enhance the efficacy of radiation therapy. Given that CRISPRi is effective in a wide range of cancer cell types [8], this work presents a conceptual and experimental framework that can be used to rapidly interrogate new targets in multiple, clinically relevant treatment combinations (e.g., synthetic lethality with traditional chemotherapy and/or radiation, or with newer classes of targeted therapeutics). The prioritization of combinatorial therapeutic targets for preclinical development is increasingly needed, particularly for malignant glioma [58-60]. While the present study focused on lncRNAs as potential therapeutic targets in glioma, the overall strategy described here could be easily adapted to screen other types of noncoding genomic elements as well as coding genes.

The interaction between radiation treatment and any particular biological target can be difficult to predict, especially when the mechanism of action of the target is not fully understood. In this study of lncRNAs, of the 467 screen hits that reduced the growth of irradiated GBM cells, only 33 hits behaved as sensitizers (having effect sizes greater with fractionated radiotherapy than without radiation). Interestingly, some lncRNA hits that reduced the growth of non-irradiated GBM cells appeared to ameliorate the effect of radiation (FIG. 3C). These results highlight the importance of unbiased functional genomics methods such as CRISPRi-based pooled screening, particularly when investigating new classes of potential therapeutic targets.

This systematic approach prioritized lncGRS-1 as a top radiation sensitizer for malignant glioma. Knockdown of lncGRS-1 with either CRISPRi or ASOs inhibited the growth of both adult and pediatric forms of malignant glioma, potentiating the therapeutic effects of clinically relevant doses of fractionated radiotherapy. Therefore, lncGRS-1 shares properties both of an essential gene that is required for normal growth and also a radiation sensitizer target.

However, despite being expressed in non-malignant brain cells, lncGRS-1 knockdown did not appear to adversely affect their viability or growth. Of particular note, ASO-mediated knockdown of lncGRS-1 in NHA—cells that proliferate as briskly as the patient-derived glioma cells—did not impair the growth of this non-malignant glial cell type, nor did lncGRS-1 knockdown affect growth of the malignant but non-glioma cell line HeLa. While the transcriptomes of GBM and DIPG cells were broadly perturbed by lncGRS-1 knockdown, only 17 genes were differentially expressed (with lncGRS-1 being the most significant) in NHAs with the same ASO-mediated knockdown. The mechanism(s) underlying this glioma-specific function of lncGRS-1 remain to be discovered, but upon lncGRS-1 knockdown, we did observe activation of the p53 signaling pathway leading to upregulation of p53 effectors such as p21, correlating with a decrease in cell cycle progression. Our observations here build importantly on previous genome-scale studies that reveal essential lncRNAs as having exquisitely cell type-specific function [8,61,62]. Based on this emerging understanding of lncRNA biology, we speculate that lncRNAs as a class are enriched for targets that have important roles selectively in malignant cells.

Modeling human glioma in mice as transplanted xenografts is time-consuming and relatively expensive, particularly for early preclinical studies intended to screen new drugs for therapeutic efficacy and potential toxicity [25]. Furthermore, certain human therapeutic targets do not exist in animal hosts—as is the case for lncGRS-1—decreasing the utility of animal models for assessing potential toxicity stemming from the ablation of such targets in normal tissue. Human organoids represent a medium-throughput platform for cancer studies [63], and the present studies demonstrate the utility of MBOs for simultaneously evaluating treatment efficacy and brain toxicity, including in the context of current radiotherapy treatment paradigms.

As a 3D tissue platform for the study of human glioma, MBOs offer certain characteristics that distinguish them from embryonic brain organoids and GBM-derived tumor organoids. In contrast to embryonic brain organoids that mimic early stages of fetal brain development, MBOs are assembled from cell populations that are more mature and postmitotic [54,55]. Because embryonic brain organoids contain a large proportion of proliferative precursor cells, radiation treatment and/or other traditional chemotherapies may not be well-tolerated by such normal but immature cells. GBM-derived tumor organoids are useful for the study of drug efficacy in 3D tissues, but because they are comprised of only tumor cells, toxicity to normal cells and therefore the therapeutic index [64] cannot be not assessed. Because of these differences, it is likely that each organoid platforms will have important utility in different pre-clinical research scenarios. As a bridge between in vitro and in vivo pre-clinical experiments intended to prioritize drug candidates and therapeutic targets, MBOs have shown utility in our study, identifying lncGRS-1 as a target for further preclinical development such as in vivo validation. One limitation of our MBO-glioma model is the absence of a complete tumor microenvironment, which includes microglia, stromal cells, tumor infiltrating lymphocytes, among other cell types [65,66]. However, recent advances in tumor organoid derivation have demonstrated preservation of syngeneic tumor infiltrating lymphocytes and stromal fibroblasts along with neoplastic cells using an air liquid interface [67]. Analogous strategies, in addition to the incorporation of vascular endothelial cells, may further augment the experimental utility of MBOs in future iterations.

4. Conclusion

There is an unmet need for cancer therapies that potentiate the therapeutic effects of radiation therapy while minimizing toxicity [59]. Radiation therapy is one of the most common treatment modalities for all cancers, and it is nearly always indicated for patients with malignant glioma [23]. The development of drug-radiotherapy combinations has generally been pursued with low-throughput, non-systematic approaches [60]. This genome scale CRISPRi-based radiation modifier screen of lncRNA loci in glioma demonstrates a strategy for revealing novel therapeutic targets in this vast, largely unexplored aspect of the noncoding genome. More broadly, it is anticipated that the coupling of large scale screening efforts with target validation in MBO models will serve as a useful framework for accelerating the development of new therapeutics (and combinations) through the preclinical research pipeline.

### REFERENCES

1. Cabili M N, et al. Integrative annotation of human large intergenic noncoding RNAs reveals global properties and specific subclasses. Genes & Development. 2011; 25:1915-27.
2. Iyer M K, et al. The landscape of long noncoding RNAs in the human transcriptome. Nature Genetics. 2015.
3. Hon C-C, et al. An atlas of human long non-coding RNAs with accurate 5′ ends. Nature. 2017; 543:199-204.
4. Rinn J L, et al. Genome regulation by long noncoding RNAs. Annu. Rev. Biochem. 2012; 81:145-66.
5. Gupta R A, et al. Long non-coding RNA HOTAIR reprograms chromatinstate to promote cancer metastasis. Nature. Nature Publishing Group; 2011; 464:1071-6.
6. Schmitt A M, et al. Long Noncoding RNAs in Cancer Pathways. Cancer Cell. 2016; 29:452-63.
7. Ji P, et al. MALAT-1, a novel noncoding RNA, and thymosin beta4 predict metastasis and survival in early-stage non-small cell lung cancer. Oncogene. 2003; 22:8031-41.
8. Liu S J, et al. CRISPRi-based genome-scale identification of functional long noncoding RNA loci in human cells. Science. 2017; 355:eaah7111.
9. Wang T, et al. Genetic screens in human cells using the CRISPR-Cas9 system. Science. 2014; 343:80-4.
10. Wang T, et al. Gene Essentiality Profiling Reveals Gene Networks and Synthetic Lethal Interactions with Oncogenic Ras. Cell. 2017; 168:890-903.e15.
11. Gilbert L A, et al. Genome-Scale CRISPR-Mediated Control of Gene Repression and Activation. Cell. 2014; 159:647-61.
12. Shalem O, et al. Genome-scale CRISPR-Cas9 knockout screening in human cells. Science. 2014; 343:84-7.
13. Yeo N C, et al. An enhanced CRISPR repressor for targeted mammalian gene regulation. Nat Meth. Nature Publishing Group; 2018; 31:230-616.
14. Konermann S, et al. Genome-scale transcriptional activation by an engineered CRISPR-Cas9 complex. Nature. 2015; 517:583-8.
15. Hart T, et al. High-Resolution CRISPR Screens Reveal Fitness Genes and Genotype-Specific Cancer Liabilities. Cell. 2015; 163:1515-26.
16. Behan F M, et al. Prioritization of cancer therapeutic targets using CRISPR-Cas9 screens. Nature. Nature Publishing Group; 2019; 568:511-6.
17. Omuro A, et al. Glioblastoma and other malignant gliomas: a clinical review. JAMA. 2013; 310:1842-50.
18. Stupp R, et al. Effects of radiotherapy with concomitant and adjuvant temozolomide versus radiotherapy alone on survival in glioblastoma in a randomised phase III study: 5-year analysis of the EORTC-NCIC trial. Lancet Oncol. 2009; 10:459-66.
19. Stupp R, et al. Radiotherapy plus concomitant and adjuvant temozolomide for glioblastoma. N Engl J Med. 2005; 352:987-96.
20. Hargrave D, et al. Diffuse brainstem glioma in children: critical review of clinical trials. Lancet Oncol. 2006; 7:241-8.
21. Kline C, et al. Reirradiation and PD-1 inhibition with nivolumab for the treatment of recurrent diffuse intrinsic pontine glioma: a single-institution experience. J. Neurooncol. Springer US; 2018; 86:1064-10.
22. Zaghloul M S, et al. Hypofractionated conformal radiotherapy for pediatric diffuse intrinsic pontine glioma (DIPG): a randomized controlled trial. Radiother Oncol. 2014; 111:35-40.
23. Barani I J, et al. Radiation Therapy of Glioblastoma. Current Understanding and Treatment of Gliomas. Cham: Springer International Publishing; 2014. pp. 49-73.
24. Schild S E, et al. The results of radiotherapy for brainstem tumors. J. Neurooncol. 1998; 40:171-7.
25. Tuveson D, et al. Cancer modeling meets human organoid technology. Science. American Association for the Advancement of Science; 2019; 364:952-5.
26. Lancaster M A, et al. Cerebral organoids model human brain development and microcephaly. Nature. Nature Publishing Group; 2013;501:373-9.
27. Kadoshima T, et al. Self-organization of axial polarity, inside-out layer pattern, and species-specific progenitor dynamics in human ES cell-derived neocortex. Proceedings of the National Academy of Sciences. National Academy of Sciences; 2013; 110:20284-9.
28. Qian X, et al. Brain-Region-Specific Organoids Using Mini-bioreactors for Modeling ZIKV Exposure. Cell. 2016; 165:1238-54.
29. Bian S, et al. Genetically engineered cerebral organoids model brain tumor formation. Nat Meth. Nature Publishing Group; 2018; 15:631-9.
30. Linkous A, et al. Modeling Patient-Derived Glioblastoma with Cerebral Organoids. Cell Reports. 2019; 26:3203-5.
31. Qi L S, et al. Repurposing CRISPR as an RNA-guided platform for sequence-specific control of gene expression. Cell. 2013; 152:1173-83.
32. Gilbert L A, et al. CRISPR-mediated modular RNA-guided regulation of transcription in eukaryotes. Cell. 2013; 154:442-51.
33. Bassik M C, et al. A systematic mammalian genetic interaction map reveals pathways underlying ricin susceptibility. Cell. 2013; 152:909-22.
34. Senra J M, et al. Inhibition of PARP-1 by olaparib (AZD2281) increases the radiosensitivity of a lung tumor xenograft. Mol. Cancer Ther. 2011; 10:1949-58.
35. Collis S J, et al. Enhanced radiation and chemotherapy-mediated cell killing of human cancer cells by small inhibitory RNA silencing of DNA repair factors. Cancer Research. 2003; 63:1550-4.
36. Tummala H, et al. ERCC6L2 Mutations Link a Distinct Bone-Marrow-Failure Syndrome to DNA Repair and Mitochondrial Function. The American Journal of Human Genetics. 2014; 94:246-56.
37. Zhang S, et al. A nonsense mutation in the DNA repair factor Hebo causes mild bone marrow failure and microcephaly. J. Exp. Med. 2016; 213:1011-28.
38. Negrini S, et al. Genomic instability—an evolving hallmark of cancer. Nat Rev Mol Cell Biol. Nature Publishing Group; 2010; 11:220-8.
39. Hanahan D, et al. Hallmarks of Cancer: The Next Generation. Elsevier Inc; 2011; 144:646-74.
40. Cho S W, et al. Promoter of lncRNA Gene PVT1 Is a Tumor-Suppressor DNA Boundary Element. Cell. 2018; 173:1398-1412.e22.
41. Frankish A, et al. GENCODE reference annotation for the human and mouse genomes. Nucleic Acids Res. 2019; 47:D766-73.
42. Ghandi M, et al. Next-generation characterization of the Cancer Cell Line Encyclopedia. Nature. 2019; 569:503-8.
43. Lennox K A, et al. Cellular localization of long non-coding RNAs affects silencing by RNAi more than by antisense oligonucleotides. Nucleic Acids Res. 2016; 44:863-77.
44. Meng L, et al. Towards a therapy for Angelman syndrome by targeting a long non-coding RNA. Nature. 2015; 518:409-12.
45. Chiriboga C A, et al. Results from a phase 1 study of nusinersen (ISIS-SMN(Rx)) in children with spinal muscular atrophy. Neurology. Wolters Kluwer Health, Inc. on behalf of the American Academy of Neurology; 2016; 86:890-7.
46. Sofroniew M V, et al. Astrocytes: biology and pathology. Acta Neuropathol. Third. 2010; 119:7-35.
47. Lee J H, et al. Human glioblastoma arises from subventricular zone cells with low-level driver mutations. Nature. 2018; 560:243-7.
48. Filbin M G, et al. Developmental and oncogenic programs in H3K27M gliomas dissected by single-cell RNA-seq. Science. American Association for the Advancement of Science; 2018; 360:331-5.
49. Sonoda Y, et al. Formation of intracranial tumors by genetically modified human astrocytes defines four pathways critical in the development of human anaplastic astrocytoma. Cancer Research. 2001; 61:4956-60.
50. Quadrato G, et al. The promises and challenges of human brain organoids as models of neuropsychiatric disease. Nat Med. Nature Publishing Group; 2016; 22:1220-8.
51. Velasco S, et al. Individual brain organoids reproducibly form cell diversity of the human cerebral cortex. Nature. 2019; 570:523-7.
52. Sloan S A, et al. Human Astrocyte Maturation Captured in 3D Cerebral Cortical Spheroids Derived from Pluripotent Stem Cells. Neuron. 2017; 95:779-790.e6.
53. Molofsky A V, et al. Astrocytes and disease: a neurodevelopmental perspective. Genes & Development. 2012; 26:891-907.
54. Krencik R, et al. Dysregulation of astrocyte extracellular signaling in Costello syndrome. Science Translational Medicine. 2015; 7:286ra66-6.
55. Krencik R, et al. Systematic Three-Dimensional Coculture Rapidly Recapitulates Interactions between Human Neurons and Astrocytes. Stem Cell Reports. 2017; 9:1745-53.
56. Wang C, et al. Scalable Production of iPSC-Derived Human Neurons to Identify Tau-Lowering Compounds by High-Content Screening. Stem Cell Reports. 2017; 9:1221-33.
57. Schoch K M, et al. Antisense Oligonucleotides: Translation from Mouse Models to Human Neurodegenerative Diseases. Neuron. 2017; 94:1056-70.
58. Prados M D, et al. Toward precision medicine in glioblastoma: the promise and the challenges. Neuro-Oncology. 2015; 17:1051-63.
59. Sharma R A, et al. Clinical development of new drug-radiotherapy combinations. Nat Rev Clin Oncol. Nature Publishing Group; 2016. pp. 627-42.
60. Ahmad S S, et al. Clinical Development of Novel Drug-Radiotherapy Combinations. Clin. Cancer Res. 2019; 25:1455-61.
61. Liu Y, et al. Genome-wide screening for functional long noncoding RNAs in human cells by Cas9 targeting of splice sites. Nat Biotechnol. Nature Publishing Group; 2018; 1656:175-210.
62. Zhu S, et al. Genome-scale deletion screening of human long non-coding RNAs using a paired-guide RNA CRISPR-Cas9 library. Nat Biotechnol. 2016; 34:1279-86.
63. van de Wetering M, et al. Prospective derivation of a living organoid biobank of colorectal cancer patients. Cell. 2015; 161:933-45.
64. Muller P Y, et al. The determination and interpretation of the therapeutic index in drug development. Nat Rev Drug Discov. 2012; 11:751-61.
65. Venteicher A S, et al. Decoupling genetics, lineages, and microenvironment in IDH-mutant gliomas by single-cell RNA-seq. Science. American Association for the Advancement of Science; 2017; 355:eaai8478.
66. Müller S, et al. Single-cell profiling of human gliomas reveals macrophage ontogeny as a basis for regional differences in macrophage activation in the tumor microenvironment. Genome Biol. BioMed Central; 2017; 18:234.
67. Neal J T, et al. Organoid Modeling of the Tumor Immune Microenvironment. Cell. 2018; 176:1972-1988.e16.
68. Horlbeck M A, et al. Compact and highly active next-generation libraries for CRISPR-mediated gene repression and activation. eLife. 2016; 5:914.
69. Liu S J, et al. Single-cell analysis of long non-coding RNAs in the developing human neocortex. Genome Biol. BioMed Central; 2016; 17:67.
70. Wysocka J, et al. Loss of HCF-1-chromatin association precedes temperature-induced growth arrest of tsBN67 cells. Molecular and Cellular Biology. 2001; 21:3820-9.
71. Kim D, et al. HISAT: a fast spliced aligner with low memory requirements. Nat Meth. 2015; 12:357-60.
72. Liao Y, et al. featureCounts: an efficient general purpose program for assigning sequence reads to genomic features. Bioinformatics. Oxford University Press; 2014; 30:923-30.
73. Anders S, et al. Differential expression analysis for sequence count data. Genome Biol. 2010; 11:R106.
74. Chen E Y, et al. Enrichr: interactive and collaborative HTMLS gene list enrichment analysis tool. BMC Bioinformatics. BioMed Central; 2013; 14:128.
75. Fernandopulle M S, et al. Transcription Factor-Mediated Differentiation of Human iPSCs into Neurons. Curr Protoc Cell Biol. 2018; 79:e51.
76. Liu, S J. Perturbation of long non-coding RNA therapeutic targets in glioma. SRA BioProject: PRJNA609239. https://www.ncbi.nlm.nih.gov/sra/PRJNA609239 (2020).

1-105. (canceled)

106. A system comprising:
a) one or a plurality of nucleic acids complementary to, or substantially complementary to, one or a plurality of lncRNAs chosen from nucleic acid sequences comprising at least about 70% sequence identity to one or a plurality of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 and SEQ ID NO: 9; and
b) a solid support onto to which the one or plurality of nucleic acids are immobilized.

107. The system of claim 106, wherein the one or plurality of nucleic acids comprises at least about 70% sequence identity to one or a plurality of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 and SEQ ID NO: 46, or a functional fragment thereof.

108. The system of claim 106, wherein the one or plurality of nucleic acids are fragments of any of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 and SEQ ID NO: 46, wherein the fragment comprises from about 10 to about 100 nucleotides.

109. A method of preparing a sample from a subject comprising:
a) isolating total RNA from the sample; and
b) analyzing the total RNA with a probe specific for one or a plurality of long noncoding RNAs (lncRNAs) comprising a nucleic acid sequence having at least about 70% sequence identity to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 and SEQ ID NO: 9.

110. A method of identifying a radiotherapy sensitizer comprising:
a) exposing a plurality of test cells to radiation, the plurality of test cells each comprising:
i) a small guide RNA (sgRNA) that targets a locus encoding a long non-coding RNA; and
ii) a nuclease-deficient sgRNA-mediated nuclease (dCas9), wherein the dCas9 comprises a dCas9 domain fused to a transcriptional modulator;
b) selecting a test cell having decreased cell proliferation as compared to a control cell treated with radiation alone; and
c) identifying the locus targeted by the sgRNA in the test cell as the radiotherapy sensitizer.

111. The method of claim 110, wherein the test cells are glioblastomas (GBM) cells.

112. The method of claim 110, wherein the dCas9 comprises an amino acid sequence having at least about 70% sequence identity to the amino acid sequence of SEQ ID NO: 12, SEQ ID NO: 13, SEQ ID NO: 14 or SEQ ID NO: 15.

113. The method of claim 110, wherein the radiation used to expose the cells is at a total dose of from about 1 Gy to about 12 Gy and delivered in about 1 to about 6 fractions.

114. The method of claim 110, further comprising the steps of:
i) obtaining a radiation modifier screen score of each test cell, wherein the radiation modifier screen score is an average phenotype of top three sgRNAs against a gene multiplied by the negative log 10(Mann-Whitney-U p value) for the gene in the test cell treated with radiation; and
ii) obtaining a growth screen score of each test cell that is not irradiated, wherein the growth screen score is an average phenotype of top three sgRNAs against the gene multiplied by the negative log 10(Mann-Whitney-U p value) for said given gene in the test cell that is not irradiated.

115. A pharmaceutical composition comprising a therapeutically effective amount of an agent that knockdowns expression of a long noncoding RNA (lncRNA) having at least about 70% sequence identity to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 and SEQ ID NO: 9 and a pharmaceutically acceptable carrier.

116. The pharmaceutical composition of claim 115, wherein the agent that knockdowns expression of the lncRNA is a miRNA, siRNA, antisense oligonucleotide (ASO), or morpholino.

117. The pharmaceutical composition of claim 115, wherein the lncRNA comprises a nucleic acid sequence having at least about 70% sequence identity to SEQ ID NO: 1.

118. The pharmaceutical composition of claim 115, wherein the agent that knockdowns expression of the lncRNA is an ASO comprising a nucleic acid sequence having at least about 70% sequence identity to SEQ ID NO: 10 or SEQ ID NO: 11.

119. A method of treating brain cancer in a subject in need thereof comprising administering to the subject a therapeutically effective amount of an agent that knockdowns expression of a lncRNA having at least about 70% sequence identity to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 and SEQ ID NO: 9.

120. The method of claim 119, wherein the agent that knockdowns expression of the lncRNA is a microRNA (miRNA), small interfering RNA (siRNA), antisense oligonucleotide (ASO), or morpholino.

121. The method of claim 120, wherein the agent is an ASO comprising a nucleic acid sequence having at least about 70% sequence identity to SEQ ID NO: 10 or SEQ ID NO: 11.

122. The method of claim 119, wherein the subject is concurrently under radiation therapy.

123. The method of claim 119, wherein administration of the agent enhances the effect of the radiation therapy as compared to administering the radiation therapy alone.

124. The method of claim 119, wherein administration of the agent is accompanied by simultaneous administration of fractionated radiation from about 1 to about 12 Gy.

125. The method of claim 119, wherein the agent is an ASO comprising a nucleic acid sequence having at least about 70% sequence identity to SEQ ID NO: 10 or SEQ ID NO: 11, and wherein the agent is administered, intravenously, intraperitoneally, intramuscularly, subcutaneously, orally, or directly into a tumor of the subject.
